SEQUENCE LISTING (56 sequences)

SEQ ID NO: 1 - RNA, 1752 nt, HOMO_SAPIENS
agggcgcagcaggccaagggggaggugcgagcguggaccugggacgggucugggcggcucucggugguuggcacggguucgcacacccauucaagcggcaggacgcacuugucuuagcaguucucgcugaccgcgcuagcugcggcuucuacgcuccggcacucugaguucaucagcaaacgcccuggcgucuguccucaccaugccuagccuuugggaccgcuucucgucgucguccaccuccucuucgcccucguccuugccccgaacucccaccccagaucggccgccgcgcucagccugggggucggcgacccgggaggagggguuugaccgcuccacgagccuggagagcucggacugcgagucccuggacagcagcaacaguggcuucgggccggaggaagacacggcuuaccuggauggggugucguugcccgacuucgagcugcucagugacccugaggaugaacacuugugugccaaccugaugcagcugcugcaggagagccuggcccaggcgcggcugggcucucgacgcccugcgcgccugcugaugccuagccaguugguaagccaggugggcaaagaacuacugcgccuggccuacagcgagccgugcggccugcggggggcgcugcuggacgucugcguggagcagggcaagagcugccacagcgugggccagcuggcacucgaccccagccuggugcccaccuuccagcugacccucgugcugcgccuggacucacgacucuggcccaagauccaggggcuguuuagcuccgccaacucucccuuccucccuggcuucagccagucccugacgcugagcacuggcuuccgagucaucaagaagaagcuguacagcucggaacagcugcucauugaggaguguugaacuucaaccugagggggccgacagugcccuccaagacagagacgacugaacuuuugggguggagacuagaggcaggagcugagggacugauuccugugguuggaaaacugaggcagccaccuaagguggaggugggggaauaguguuucccaggaagcucauugaguugugugcggguggcugugcauuggggacacauaccccucaguacuguagcaugaaacaaaggcuuaggggccaacaaggcuuccagcuggauguguguguagcauguaccuuauuauuuuuguuacugacaguuaacaguggugugacauccagagagcagcugggcugcucccgccccagcccggcccagggugaaggaagaggcacgugcuccucagagcagccggagggaggggggaggucggaggucguggaggugguuuguguaucuuacuggucugaagggaccaaguguguuuguuguuuguuuuguaucuuguuuuucugaucggagcaucacuacugaccuguuguaggcagcuaucuuacagacgcaugaauguaagaguaggaaggggugggugucagggaucacuugggaucuuugacacuugaaaaauuacaccuggcagcugcguuuaagccuucccccaucguguacugcagaguugagcuggcaggggaggggcugagagggugggggcuggaaccccuccccgggaggagugccaucugggucuuccaucuagaacuguuuacaugaagauaagauacucacuguucaugaauacacuugauguucaaguauuaagaccuaugcaauauuuuuuacuuuucuaauaaacauguuuguuaaaacaguu

SEQ ID NO: 2 - RNA, 4242 nt, HOMO_SAPIENS
cuuuugucuguccgccgagcaccccacuucaccccauuggaccgcgcggccgccgcuagagcucugcgccugcgcacgcaccgggccggggacuggguggccuggugugugggcgcggcagggcgcaggcgcaggcgcagugugcguccgcgucugaggggagggaugugggggaagcgacggcccccgguuuguuugggcugugggcggugcgcagcggagagcccgggaaaagcgggaaauggcggcgccgagcgcggggucuugguccaccuuccagcacaaggagcugauggccgcugacaggggacgcaggauauugggaguguguggcaugcauccucaucaucaggaaacucuaaaaaagaaccgaguggugcuagccaaacagcuguuguugagcgaauuguuagaacaucuucuggagaaggacaucaucaccuuggaaaugagggagcucauccaggccaaagugggcaguuucagccagaauguggaacuccucaacuugcugccuaagagggguccccaagcuuuugaugccuucugugaagcacugagggagaccaagcaaggccaccuggaggauauguugcucaccacccuuucugggcuucagcauguacucccaccguugagcugugacuacgacuugagucucccuuuuccggugugugaguccuguccccuuuacaagaagcuccgccugucgacagauacuguggaacacucccuagacaauaaagaugguccugucugccuucaggugaagccuugcacuccugaauuuuaucaaacacacuuccagcuggcauauagguugcagucucggccucguggccuagcacugguguugagcaaugugcacuucacuggagagaaagaacuggaauuucgcucuggaggggauguggaccacaguacucuagucacccucuucaagcuuuugggcuaugacguccauguucuaugugaccagacugcacaggaaaugcaagagaaacugcagaauuuugcacaguuaccugcacaccgagucacggacuccugcaucguggcacuccucucgcaugguguggagggcgccaucuaugguguggaugggaaacugcuccagcuccaagagguuuuucagcucuuugacaacgccaacugcccaagccuacagaacaaaccaaaaauguucuucauccaggccugccguggagaugagacugaucgugggguugaccaacaagauggaaagaaccacgcaggauccccugggugcgaggagagugaugccgguaaagaaaaguugccgaagaugagacugcccacgcgcucagacaugauaugcggcuaugccugccucaaagggacugccgccaugcggaacaccaaacgagguuccugguacaucgaggcucuugcucaaguguuuucugagcgggcuugugauaugcacguggccgacaugcugguuaaggugaacgcacuuaucaaggaucgggaagguuaugcuccuggcacagaauuccaccggugcaaggagaugucugaauacugcagcacucugugccgccaccucuaccuguucccaggacacccucccacaugaugucaccuccccaucauccacgccaaguggaagccacuggaccacaggaggugugauagagccuuugaucuucaggaugcacgguuucuguucugcccccucagggaugugggaaucucccagacuuguuuccugugcccaucaucucugccuuugagugugggacuccaggccagcuccuuuucugugaagcccuuugccuguagagccagccuugguuggaccuauugccaggaauguuucagcugcaguugaagagccugacaagugaaguuguaaacacagugugguuauggggagagggcauauaaauuccccauauuuguguucaguuccagcuuuuguagauggcacuuuagugauugcuuuuauuacauuaguuaagaugucugagagaccaucuccuaucuuuuauuucauucauauccuccgcccuuuuuguccuagagugagaguuuggaagguguccaaauuuaauguagacauuaucuuuuggcucugaagaagcaaacaugacuagagacgcaccuugcugcaguguccagaagcggccugugcguucccuucaguacugcagcgccacccaguggaaggacacucuuggcucguuugggcucaaggcaccgcagccugucagccaacauugccuugcauuuguaccuuauugaucuuugcccauggaagucucaaagaucuuucguugguuguuucucugagcuuuguuacugaaaugagccucguggggagcaucagagaaggccaggaagaaugguguguuucccuagacucuguaaccaccucucugucuuuuuccuuccugagaaacguccaucucucucccuuacuauucccacuuucauucaaucaaccugcacuucauaucuagauuucuagaaaagcuuccuagcuuaucucccugcuucauaucucucccuucuuuaccuucauuucauccuguuggcugcugccaccaaaucugucuagaauccugcuuuacaggaucauguaaaugcucaaagauguaauguaguucuuuguuccugcuuucucuuucaguauuaaacucuccuuugauauuauguggcuuuuauuucagugccauacauguuauuguuuucaaccuagaaaccuuuaucccugcuuaucugaaacuucccaacuucccuguucuuuaagacuuuuuuuuuuuuuuuuuuuuuuuuugagacagagucucgcucugucgcccaggcuggagggcaguggcacgaucucagcucacugcaagcuccaacucccggguucacgccauucuccugccucagccuuccaaguagcugggacuacaggugcccgccaccgugcccggcuaauuuuuuuguauuuuuaguagagacaggguuucaccauguuagccgggauggucuugaucuccugaccucaugauccacccaccucagccucccaaaguguugggauuacaggcgugagccacugcgcccgggcaagaccuuuuuuuaaaaaaaaaaaaaaaaaaacuuccauucuuucuuccuccagucuguucucacauaacagaguaguuuugguuuuuaauuuuuuuugguuguuugcuguuuuuuguuuuuuaaggugaguucucacuauguuucucagacuggucucgaacuccuggccucaagccaucuucccgccucagccucucaaauagcugggcuuacaggcaugagccaccacaccuggccaggauuugguuguuuaaauauaaaucugaucaccccccugcuuagaacccuucugcuuucuauuaccccucauuuaaaauguaaacucuucaccuugguuuaugagaacugguucuugccuuccccuugaaccucauuaaauggugauuucuugcuaagcuccagcccgaguggucuccucucagcuucuaauuuugugcucuuuccugcccuuuuccugggccuucucagcucuccacccccaccacucuugacucaggugguguccuucuuccucaagucuugacaauucccgggcccuucagucccugagcagucuacuucugugucugucaccacaucuugucuuuuccccucauugcauuuauugcaguuuauauauaugcuacuuuuacuuguucauuucugucuccccuaccaggcuguaaaugagggcagaaaccuuguuuguuuuauucaccaucauguaccaagugcuuggcacauagugggccuucauuaaauguuuguugaauaaaagagggaagaaggcaagccaaccuuagcuacaauccuaccuuuugauaaaauguuccuuuugacaauauacacggauuauuauuuguacuuuguuuuuccauguguuuugcuuuuauccacuggcauuuuuagcuccuugaagacauaucaugugugagauaacuuccuucacaucucccauggucccuagcaaaaugcuaggccuguaguagucaaggugcucaauaaauauuuguuugggugguuugugagccuugcugccaaguccugccuuugggucgacauaguauggaaguauuugagagagagaaccuuuccacucccacugccaggauuuuguauugccaucgggugccaaauaaaugcucauauuuauuacugaaaaaaaaaaaaaaaaaa

SEQ ID NO: 3 - RNA, 4057 nt, HOMO_SAPIENS
agaggaagagaacgauuuaaggagcgaauacuacugguaaacuaauggaagaaaucugcugcaccacuggauauugggaguguguggcaugcauccucaucaucaggaaacucuaaaaaagaaccgaguggugcuagccaaacagcuguuguugagcgaauuguuagaacaucuucuggagaaggacaucaucaccuuggaaaugagggagcucauccaggccaaagugggcaguuucagccagaauguggaacuccucaacuugcugccuaagagggguccccaagcuuuugaugccuucugugaagcacugagggagaccaagcaaggccaccuggaggauauguugcucaccacccuuucugggcuucagcauguacucccaccguugagcugugacuacgacuugagucucccuuuuccggugugugaguccuguccccuuuacaagaagcuccgccugucgacagauacuguggaacacucccuagacaauaaagaugguccugucugccuucaggugaagccuugcacuccugaauuuuaucaaacacacuuccagcuggcauauagguugcagucucggccucguggccuagcacugguguugagcaaugugcacuucacuggagagaaagaacuggaauuucgcucuggaggggauguggaccacaguacucuagucacccucuucaagcuuuugggcuaugacguccauguucuaugugaccagacugcacaggaaaugcaagagaaacugcagaauuuugcacaguuaccugcacaccgagucacggacuccugcaucguggcacuccucucgcaugguguggagggcgccaucuaugguguggaugggaaacugcuccagcuccaagagguuuuucagcucuuugacaacgccaacugcccaagccuacagaacaaaccaaaaauguucuucauccaggccugccguggaggugcuauuggaucccuugggcaccuccuucuguucacugcugccaccgccucucuugcucuaugagacugaucgugggguugaccaacaagauggaaagaaccacgcaggauccccugggugcgaggagagugaugccgguaaagaaaaguugccgaagaugagacugcccacgcgcucagacaugauaugcggcuaugccugccucaaagggacugccgccaugcggaacaccaaacgagguuccugguacaucgaggcucuugcucaaguguuuucugagcgggcuugugauaugcacguggccgacaugcugguuaaggugaacgcacuuaucaaggaucgggaagguuaugcuccuggcacagaauuccaccggugcaaggagaugucugaauacugcagcacucugugccgccaccucuaccuguucccaggacacccucccacaugaugucaccuccccaucauccacgccaaguggaagccacuggaccacaggaggugugauagagccuuugaucuucaggaugcacgguuucuguucugcccccucagggaugugggaaucucccagacuuguuuccugugcccaucaucucugccuuugagugugggacuccaggccagcuccuuuucugugaagcccuuugccuguagagccagccuugguuggaccuauugccaggaauguuucagcugcaguugaagagccugacaagugaaguuguaaacacagugugguuauggggagagggcauauaaauuccccauauuuguguucaguuccagcuuuuguagauggcacuuuagugauugcuuuuauuacauuaguuaagaugucugagagaccaucuccuaucuuuuauuucauucauauccuccgcccuuuuuguccuagagugagaguuuggaagguguccaaauuuaauguagacauuaucuuuuggcucugaagaagcaaacaugacuagagacgcaccuugcugcaguguccagaagcggccugugcguucccuucaguacugcagcgccacccaguggaaggacacucuuggcucguuugggcucaaggcaccgcagccugucagccaacauugccuugcauuuguaccuuauugaucuuugcccauggaagucucaaagaucuuucguugguuguuucucugagcuuuguuacugaaaugagccucguggggagcaucagagaaggccaggaagaaugguguguuucccuagacucuguaaccaccucucugucuuuuuccuuccugagaaacguccaucucucucccuuacuauucccacuuucauucaaucaaccugcacuucauaucuagauuucuagaaaagcuuccuagcuuaucucccugcuucauaucucucccuucuuuaccuucauuucauccuguuggcugcugccaccaaaucugucuagaauccugcuuuacaggaucauguaaaugcucaaagauguaauguaguucuuuguuccugcuuucucuuucaguauuaaacucuccuuugauauuauguggcuuuuauuucagugccauacauguuauuguuuucaaccuagaaaccuuuaucccugcuuaucugaaacuucccaacuucccuguucuuuaagacuuuuuuuuuuuuuuuuuuuuuuuuugagacagagucucgcucugucgcccaggcuggagggcaguggcacgaucucagcucacugcaagcuccaacucccggguucacgccauucuccugccucagccuuccaaguagcugggacuacaggugcccgccaccgugcccggcuaauuuuuuuguauuuuuaguagagacaggguuucaccauguuagccgggauggucuugaucuccugaccucaugauccacccaccucagccucccaaaguguugggauuacaggcgugagccacugcgcccgggcaagaccuuuuuuuaaaaaaaaaaaaaaaaaaacuuccauucuuucuuccuccagucuguucucacauaacagaguaguuuugguuuuuaauuuuuuuugguuguuugcuguuuuuuguuuuuuaaggugaguucucacuauguuucucagacuggucucgaacuccuggccucaagccaucuucccgccucagccucucaaauagcugggcuuacaggcaugagccaccacaccuggccaggauuugguuguuuaaauauaaaucugaucaccccccugcuuagaacccuucugcuuucuauuaccccucauuuaaaauguaaacucuucaccuugguuuaugagaacugguucuugccuuccccuugaaccucauuaaauggugauuucuugcuaagcuccagcccgaguggucuccucucagcuucuaauuuugugcucuuuccugcccuuuuccugggccuucucagcucuccacccccaccacucuugacucaggugguguccuucuuccucaagucuugacaauucccgggcccuucagucccugagcagucuacuucugugucugucaccacaucuugucuuuuccccucauugcauuuauugcaguuuauauauaugcuacuuuuacuuguucauuucugucuccccuaccaggcuguaaaugagggcagaaaccuuguuuguuuuauucaccaucauguaccaagugcuuggcacauagugggccuucauuaaauguuuguugaauaaaagagggaagaaggcaagccaaccuuagcuacaauccuaccuuuugauaaaauguuccuuuugacaauauacacggauuauuauuuguacuuuguuuuuccauguguuuugcuuuuauccacuggcauuuuuagcuccuugaagacauaucaugugugagauaacuuccuucacaucucccauggucccuagcaaaaugcuaggccuguaguagucaaggugcucaauaaauauuuguuugggugguuugugagccuugcugccaaguccugccuuugggucgacauaguauggaaguauuugagagagagaaccuuuccacucccacugccaggauuuuguauugccaucgggugccaaauaaaugcucauauuuauuacugaaaaaaaaaaaaaaaaaa

SEQ ID NO: 4 - RNA, 4024 nt, HOMO_SAPIENS
cuuuugucuguccgccgagcaccccacuucaccccauuggaccgcgcggccgccgcuagagcucugcgccugcgcacgcaccgggccggggacuggguggccuggugugugggcgcggcagggcgcaggcgcaggcgcagugugcguccgcgucugaggggagggaugugggggaagcgacggcccccgguuuguuugggcugugggcggugcgcagcggagagcccgggaaaagcgggaaauggcggcgccgagcgcggggucuugguccaccuuccagcacaaggagcugauggccgcugacaggggacgcaggauauugggaguguguggcaugcauccucaucaucaggaaacucuaaaaaagaaccgaguggugcuagccaaacagcuguuguugagcgaauuguuagaacaucuucuggagaaggacaucaucaccuuggaaaugagggagcucauccaggccaaagugggcaguuucagccagaauguggaacuccucaacuugcugccuaagagggguccccaagcuuuugaugccuucugugaagccuugcacuccugaauuuuaucaaacacacuuccagcuggcauauagguugcagucucggccucguggccuagcacugguguugagcaaugugcacuucacuggagagaaagaacuggaauuucgcucuggaggggauguggaccacaguacucuagucacccucuucaagcuuuugggcuaugacguccauguucuaugugaccagacugcacaggaaaugcaagagaaacugcagaauuuugcacaguuaccugcacaccgagucacggacuccugcaucguggcacuccucucgcaugguguggagggcgccaucuaugguguggaugggaaacugcuccagcuccaagagguuuuucagcucuuugacaacgccaacugcccaagccuacagaacaaaccaaaaauguucuucauccaggccugccguggagaugagacugaucgugggguugaccaacaagauggaaagaaccacgcaggauccccugggugcgaggagagugaugccgguaaagaaaaguugccgaagaugagacugcccacgcgcucagacaugauaugcggcuaugccugccucaaagggacugccgccaugcggaacaccaaacgagguuccugguacaucgaggcucuugcucaaguguuuucugagcgggcuugugauaugcacguggccgacaugcugguuaaggugaacgcacuuaucaaggaucgggaagguuaugcuccuggcacagaauuccaccggugcaaggagaugucugaauacugcagcacucugugccgccaccucuaccuguucccaggacacccucccacaugaugucaccuccccaucauccacgccaaguggaagccacuggaccacaggaggugugauagagccuuugaucuucaggaugcacgguuucuguucugcccccucagggaugugggaaucucccagacuuguuuccugugcccaucaucucugccuuugagugugggacuccaggccagcuccuuuucugugaagcccuuugccuguagagccagccuugguuggaccuauugccaggaauguuucagcugcaguugaagagccugacaagugaaguuguaaacacagugugguuauggggagagggcauauaaauuccccauauuuguguucaguuccagcuuuuguagauggcacuuuagugauugcuuuuauuacauuaguuaagaugucugagagaccaucuccuaucuuuuauuucauucauauccuccgcccuuuuuguccuagagugagaguuuggaagguguccaaauuuaauguagacauuaucuuuuggcucugaagaagcaaacaugacuagagacgcaccuugcugcaguguccagaagcggccugugcguucccuucaguacugcagcgccacccaguggaaggacacucuuggcucguuugggcucaaggcaccgcagccugucagccaacauugccuugcauuuguaccuuauugaucuuugcccauggaagucucaaagaucuuucguugguuguuucucugagcuuuguuacugaaaugagccucguggggagcaucagagaaggccaggaagaaugguguguuucccuagacucuguaaccaccucucugucuuuuuccuuccugagaaacguccaucucucucccuuacuauucccacuuucauucaaucaaccugcacuucauaucuagauuucuagaaaagcuuccuagcuuaucucccugcuucauaucucucccuucuuuaccuucauuucauccuguuggcugcugccaccaaaucugucuagaauccugcuuuacaggaucauguaaaugcucaaagauguaauguaguucuuuguuccugcuuucucuuucaguauuaaacucuccuuugauauuauguggcuuuuauuucagugccauacauguuauuguuuucaaccuagaaaccuuuaucccugcuuaucugaaacuucccaacuucccuguucuuuaagacuuuuuuuuuuuuuuuuuuuuuuuuugagacagagucucgcucugucgcccaggcuggagggcaguggcacgaucucagcucacugcaagcuccaacucccggguucacgccauucuccugccucagccuuccaaguagcugggacuacaggugcccgccaccgugcccggcuaauuuuuuuguauuuuuaguagagacaggguuucaccauguuagccgggauggucuugaucuccugaccucaugauccacccaccucagccucccaaaguguugggauuacaggcgugagccacugcgcccgggcaagaccuuuuuuuaaaaaaaaaaaaaaaaaaacuuccauucuuucuuccuccagucuguucucacauaacagaguaguuuugguuuuuaauuuuuuuugguuguuugcuguuuuuuguuuuuuaaggugaguucucacuauguuucucagacuggucucgaacuccuggccucaagccaucuucccgccucagccucucaaauagcugggcuuacaggcaugagccaccacaccuggccaggauuugguuguuuaaauauaaaucugaucaccccccugcuuagaacccuucugcuuucuauuaccccucauuuaaaauguaaacucuucaccuugguuuaugagaacugguucuugccuuccccuugaaccucauuaaauggugauuucuugcuaagcuccagcccgaguggucuccucucagcuucuaauuuugugcucuuuccugcccuuuuccugggccuucucagcucuccacccccaccacucuugacucaggugguguccuucuuccucaagucuugacaauucccgggcccuucagucccugagcagucuacuucugugucugucaccacaucuugucuuuuccccucauugcauuuauugcaguuuauauauaugcuacuuuuacuuguucauuucugucuccccuaccaggcuguaaaugagggcagaaaccuuguuuguuuuauucaccaucauguaccaagugcuuggcacauagugggccuucauuaaauguuuguugaauaaaagagggaagaaggcaagccaaccuuagcuacaauccuaccuuuugauaaaauguuccuuuugacaauauacacggauuauuauuuguacuuuguuuuuccauguguuuugcuuuuauccacuggcauuuuuagcuccuugaagacauaucaugugugagauaacuuccuucacaucucccauggucccuagcaaaaugcuaggccuguaguagucaaggugcucaauaaauauuuguuugggugguuugugagccuugcugccaaguccugccuuugggucgacauaguauggaaguauuugagagagagaaccuuuccacucccacugccaggauuuuguauugccaucgggugccaaauaaaugcucauauuuauuacugaaaaaaaaaaaaaaaaaa

SEQ ID NO: 5 - RNA, 2649 nt, HOMO_SAPIENS
cuauaaaguucucgcugccccggaggcuaugcuacugcgaggagccggcgcaggguggcccgggaggggugagcagggugccgcuggcugcuggggucugcaggucaccgaguccccaggagaggggacuccuaagaagccaccugccuguguuuacccggcagcgagcgcgcaggcccccgcgaacuccuggcagcgcucaggaaaggccguugcgccucgcgaaggaaacagagccguugaccaugguugcaacuggcaguuugagcagcaagaacccggccagcauuucagaauugcuggacuguggcuaucacccagagagccugcuaagugauuuugacuacugggauuauguuguuccugaacccaaccucaacgagguaauauuugaggaaucaacuugccagaauuugguuaaaaugcuggagaacugucuguccaaaucaaagcaaacuaaacuugguugcucaaagguccuugucccugagaaacugacccagagaauugcucaagauguccugcggcuuuccucaacggagcccugcggcuugcgagguuguguuaugcacgugaacuuggaaauugaaaauguauguaaaaagcuggauaggauugugugugauucuagcgucguaccuacuuuugagcuuacacuuguguuuaagcaggagaacugcucauggacuagcuucagggacuuuuucuuuaguagaggucgcuucuccucugguuucaggagaacucugauccucagcucaggauuucgacuuguuaagaaaaaacuuuacucacugauuggaacaacagugauugaaggguccuaaaaagggaaaauauauaaagauuauuucaugauuggguaguaaaacuauucagcuagucagcuaaagucauuuguaguuugccccaccugcccuaaauaagaaaccccaaauguagucucuuuucuuucuguguuucacauucauagcaacugcagcuaacaggcugauuuucuggccuuuggagaagugauucaaaauaguguagauuuucugcauagaucccauuuuuguacagaauugaaugggauggaauagguaagcaaaaguagaagcccauuugaguuuuacauuugauuccacaauuugguuucagguaggcuugguaauagacuauauaaaccagauuugccuauuuugauuuucauauggcuuuuuuuucuguaaguuuucagaggauuuuuuaaaucacagaaucauacuaaaugauauuuagccuaucaaaacuuccaaaagcccacaccaccaguuccugacucaaauuugaaggguuuuuagacaggaggguaggauuaaguaggugaguuuaauuaaagcuuaacccuagguaagaguaaaugagaaauauuacggcaauaauggaacugcuucacuguuucuuggugacuuccucacucuaauguuuuaaagaggcaacaaaagcuuguggugccauuucaguaaccacgguguuguuuuagaugccuuuauaagcucaguuuccccuguucuuaaguguugaauacugucuuuaaacuagaaaaaugcaaaauauugaacugauauuuuuguguguaguugauuacucuuccauugagugaaugaugaauaccugugaggauaggaaauuaguucugagaucuagucccucucugauucacuuaguaaucuauccucuuuucaguauuacaugugcuuaaucucagaugaaccauuucaccauggcaguguuaucucaucucugggcuuuucugggaauugaaguaucucuccuuaaccccaauugucaaggguaguagcuguauacuaccacuuugaauuauugaaacgggucaauuuacgaagucugcauuggcuauggagauaugguuuauaguacagccuagagaaugaaacucaccguccagauaaccaugcaugcacccagauuuuuuccaccuuggauaccugucacuagggaauaauaaaggccugauuuuuugucuuauuccaacuaaguagaucauuaucucuuuccuuuuuuauguuaaugagagaauuuagccuccacucaacaauguucaauucagcaaggcuuucauauccuugcugugggucguggauaaggagcuuauucagguuuccugcccuagcuauuagcuccacuucacaugcuggagaccggcguagggacagauguauucauccugguguuacugaaaaacaggugugauccuguuacugauacuauaagugaccuaaaaugucacuguucaaauuagccaguguucuaacaaacuaaacucuucaaaugcuuggaaagauacuacaaagccaaucuuuauagaauugggccaagauaaaucuauguuguuuugcauggcuauuguuaagcuccaaagguucacuguguuucugccgcuguccuggaguugucaccacugacugggcaaggcuucuugggcauggauguagaacuguuguccuuuucccacuaacaguuaucuuugacucucuugccuguuaugcuuacaaaauggugauggcuuauggaaggcuguuaaauuaauauuccuguuaaaggaaauuaaaguuugucuauuuuugacaauaaaacauuauauauuuuuaaaaaaaaaaaaaaaaa

SEQ ID NO: 6 - RNA, 1926 nt, HOMO_SAPIENS
guggaugagcugugagugcgcgcgcgugcgcggggccgcgaccugugccggcucgagcccgcugggcacucggaggcgcgcacgucguuccccgcccucccgccgccgcccgcccucgcucucucgcgcuacccucccgccgcccgcgguccuccgucgguucucucguuaguccacggucuggucuucagcuacccgccuucgucuccgaguuugcgacucgcggaccggcguccccggcgcgaagaggcuggacucggauucguugccugagcaauggcugccauccggaagaaacuggugauuguuggugauggagccuguggaaagacaugcuugcucauagucuucagcaaggaccaguucccagagguguaugugcccacaguguuugagaacuauguggcagauaucgagguggauggaaagcagguagaguuggcuuugugggacacagcugggcaggaagauuaugaucgccugaggccccucuccuacccagauaccgauguuauacugauguguuuuuccaucgacagcccugauaguuuagaaaacaucccagaaaaguggaccccagaagucaagcauuucugucccaacgugcccaucauccugguugggaauaagaaggaucuucggaaugaugagcacacaaggcgggagcuagccaagaugaagcaggagccggugaaaccugaagaaggcagagauauggcaaacaggauuggcgcuuuuggguacauggaguguucagcaaagaccaaagauggagugagagagguuuuugaaauggcuacgagagcugcucugcaagcuagacgugggaagaaaaaaucugggugccuugucuugugaaaccuugcugcaagcacagcccuuaugcgguuaauuuugaagugcuguuuauuaaucuuaguguaugauuacuggccuuuuucauuuaucuauaauuuaccuaagauuacaaaucagaagucaucuugcuaccaguauuuagaagccaacuaugauuauuaacgauguccaacccgucuggcccaccaggguccuuuugacacugcucuaacagcccuccucugcacucccaccugacacaccaggcgcuaauucaaggaauuucuuaacuucuugcuucuuucuagaaagagaaacaguugguaacuuuugugaauuaggcuguaacuacuuuauaacuaacauguccugccuauuaucugucagcugcaagguacucuggugagucaccacuucagggcuuuacuccguaacagauuuuguuggcauagcucuggggugggcaguuuuuugaaaaugggcucaaccagaaaagcccaaguucaugcagcuguggcagaguuacaguucugugguuucauguuaguuaccuuauaguuacuguguaauuagugccacuuaauguauguuaccaaaaauaaauauaucuaccccagacuagauguaguauuuuuuguauaauuggauuuccuaauacugucauccucaaagaaaguguauugguuuuuuaaaaaagaaaguguauuuggaaauaaagucagauggaaaauucauuuuuuaaauucccguuuugucacuuuuucugauaaaagauggccauauuaccccuuuucggccccauguaucucaguaccccauggagcugggcuaaguaaauaggaauugguuucacgccugaggcaauuagacacuuuggaagauggcauaaccugucucaccuggacuuaagcaucuggcucuaauucacagugcucuuuucuccucacuguauccagguucccucccagaggagccaccaguucucauggguggcacucagucucucuucucuccagcugacuaaacuuuuuuucuguaccaguuaauuuuuccaacuacuaauagaauaaaggcaguuuucuaaaaaaa

SEQ ID NO: 7 - RNA, 19 nt, ARTIFICIAL
  Chemically synthesized
agcugcaucagguuggcac

SEQ ID NO: 8 - RNA, 19 nt, ARTIFICIAL
  Chemically synthesized
gugccaaccugaugcagcu

SEQ ID NO: 9 - RNA, 19 nt, ARTIFICIAL
  Chemically synthesized
agcugcaucagguuggcac

SEQ ID NO: 10 - RNA, 19 nt, ARTIFICIAL
  Chemically synthesized
gugccaaccugaugcagcu

SEQ ID NO: 11 - RNA, 19 nt, ARTIFICIAL
  Chemically synthesized
aggaguuccacauucuggc

SEQ ID NO: 12 - RNA, 19 nt, ARTIFICIAL
  Chemically synthesized
gccagaauguggaacuccu

SEQ ID NO: 13 - RNA, 19 nt, ARTIFICIAL
  Chemically synthesized
aggaguuccacauucuggc

SEQ ID NO: 14 - RNA, 19 nt, ARTIFICIAL
  Chemically synthesized
gccagaauguggaacuccu

SEQ ID NO: 15 - RNA, 19 nt, ARTIFICIAL
  Chemically synthesized
uucuagauggaagacccag

SEQ ID NO: 16 - RNA, 19 nt, ARTIFICIAL
  Chemically synthesized
cugggucuuccaucuagaa

SEQ ID NO: 17 - RNA, 19 nt, ARTIFICIAL
  Chemically synthesized
uugaacaucaaguguauuc

SEQ ID NO: 18 - RNA, 19 nt, ARTIFICIAL
  Chemically synthesized
gaauacacuugauguucaa

SEQ ID NO: 19 - RNA, 19 nt, ARTIFICIAL
  Chemically synthesized
aaaaauauugcauaggucu

SEQ ID NO: 20 - RNA, 19 nt, ARTIFICIAL
  Chemically synthesized
agaccuaugcaauauuuuu

SEQ ID NO: 21 - RNA, 19 nt, ARTIFICIAL
  Chemically synthesized
uacuugaacaucaagugua

SEQ ID NO: 22 - RNA, 19 nt, ARTIFICIAL
  Chemically synthesized
uacacuugauguucaagua

SEQ ID NO: 23 - RNA, 19 nt, ARTIFICIAL
  Chemically synthesized
uuuguuucaugcuacagua

SEQ ID NO: 24 - RNA, 19 nt, ARTIFICIAL
  Chemically synthesized
uacuguagcaugaaacaaa

SEQ ID NO: 25 - RNA, 19 nt, ARTIFICIAL
  Chemically synthesized
guuucaugcuacaguacug

SEQ ID NO: 26 - RNA, 19 nt, ARTIFICIAL
  Chemically synthesized
caguacuguagcaugaaac

SEQ ID NO: 27 - RNA, 19 nt, ARTIFICIAL
  Chemically synthesized
ucaugcuacaguacugagg

SEQ ID NO: 28 - RNA, 19 nt, ARTIFICIAL
  Chemically synthesized
ccucaguacuguagcauga

SEQ ID NO: 29 - RNA, 19 nt, ARTIFICIAL
  Chemically synthesized
uucaugcuacaguacugag

SEQ ID NO: 30 - RNA, 19 nt, ARTIFICIAL
  Chemically synthesized
cucaguacuguagcaugaa

SEQ ID NO: 31 - RNA, 19 nt, ARTIFICIAL
  Chemically synthesized
aggaguuccacauucuggc

SEQ ID NO: 32 - RNA, 19 nt, ARTIFICIAL
  Chemically synthesized
gccagaauguggaacuccu

SEQ ID NO: 33 - RNA, 19 nt, ARTIFICIAL
  Chemically synthesized
ugauaaaauucaggagugc

SEQ ID NO: 34 - RNA, 19 nt, ARTIFICIAL
  Chemically synthesized
gcacuccugaauuuuauca

SEQ ID NO: 35 - RNA, 19 nt, ARTIFICIAL
  Chemically synthesized
ucucuugcauuuccugugc

SEQ ID NO: 36 - RNA, 19 nt, ARTIFICIAL
  Chemically synthesized
gcacaggaaaugcaagaga

SEQ ID NO: 37 - RNA, 19 nt, ARTIFICIAL
  Chemically synthesized
acgugcauaucacaagccc

SEQ ID NO: 38 - RNA, 19 nt, ARTIFICIAL
  Chemically synthesized
gggcuugugauaugcacgu

SEQ ID NO: 39 - RNA, 19 nt, ARTIFICIAL
  Chemically synthesized
uuauuagccacucauuagg

SEQ ID NO: 40 - RNA, 19 nt, ARTIFICIAL
  Chemically synthesized
ccuaaugaguggcuaauaa

SEQ ID NO: 41 - RNA, 19 nt, ARTIFICIAL
  Chemically synthesized
uuauuagccacucauuagg

SEQ ID NO: 42 - RNA, 19 nt, ARTIFICIAL
  Chemically synthesized
ccuaaugaguggcuaauaa

SEQ ID NO: 43 - RNA, 19 nt, ARTIFICIAL
  Chemically synthesized
ucuugagcaauucucuggg

SEQ ID NO: 44 - RNA, 19 nt, ARTIFICIAL
  Chemically synthesized
cccagagaauugcucaaga

SEQ ID NO: 45 - RNA, 19 nt, ARTIFICIAL
  Chemically synthesized
ucuugagcaauucucuggg

SEQ ID NO: 46 - RNA, 19 nt, ARTIFICIAL
  Chemically synthesized
cccagagaauugcucaaga

SEQ ID NO: 47 - RNA, 21 nt, ARTIFICIAL
  Chemically synthesized
ggugcgcuccuggacguagcc

SEQ ID NO: 48 - RNA, 21 nt, ARTIFICIAL
  Chemically synthesized
ggcuacguccaggagcgcacc

SEQ ID NO: 49 - RNA, 21 nt, ARTIFICIAL
  Chemically synthesized
ggugcgcuccuggacguagcc

SEQ ID NO: 50 - RNA, 21 nt, ARTIFICIAL
  Chemically synthesized
ggcuacguccaggagcgcacc

SEQ ID NO: 51 - RNA, 19 nt, ARTIFICIAL
  Chemically synthesized
aaacuaucagggcugucga

SEQ ID NO: 52 - RNA, 19 nt, ARTIFICIAL
  Chemically synthesized
ucgacagcccugauaguuu

SEQ ID NO: 53 - RNA, 19 nt, ARTIFICIAL
  Chemically synthesized
aaacuaucagggcugucga

SEQ ID NO: 54 - RNA, 19 nt, ARTIFICIAL
  Chemically synthesized
ucgacagcccugauaguuu

SEQ ID NO: 55 - RNA, 19 nt, Artificial
  Chemically synthesized
gcaucgcgcguaauuuagu

SEQ ID NO: 56 - RNA, 19 nt, Artificial
  Chemically synthesized
acuaaauuacgcgcgaugc

(19) United States
(12) Patent Application Publication
(10) Pub. No.: US 2014/0323549 A1
(43) Pub. Date: Oct. 30, 2014

(54) METHODS AND COMPOSITIONS FOR TREATING DISEASES, DISORDERS OR INJURY OF THE NERVOUS SYSTEM

(71) Applicant: QUARK PHARMACEUTICALS, INC., Fremont, CA (US)

(72) Inventor: Elena Feinstein, Rehovot (IL)

(73) Assignee: QUARK PHARMACEUTICALS, INC., Fremont, CA (US)

(21) Appl. No.: 14/356,156

(22) PCT Filed: Nov. 8, 2012
(86) PCT No.: PCT/US2012/064015

§ 371 Date: May 4, 2014

Related U.S. Application Data
(60) Provisional application No. 61/556,913, filed on Nov. 8, 2011.
(60) Provisional application No. 61/641,307, filed on May 2, 2012.

(51) Int. Cl. C12N 15/113; A61K 31/713

(52) U.S. Cl. CPC C12N 15/1136; C12N 15/1137; A61K 31/713
USPC 514/44 A; 536/24.5

(57) ABSTRACT

Disclosed herein are compositions and methods of use thereof for treating diseases, disorders and injuries of the nervous system, comprising a combination of a RTP801 or RTP801L inhibitor, and a Casp2 inhibitor.

### RELATED APPLICATIONS

This application claims the benefit of U.S. Provisional Application Ser. No. 61/556,913 filed Nov. 8, 2011 and of U.S. Provisional Application Ser. No. 61/641,307 filed May 2, 2012, both entitled “Methods and Compositions for Treating Diseases, Disorders or Injury of the Eye and CNS” and incorporated herein by reference in their entirety and for all purposes.

### SEQUENCE LISTING

This application incorporates-by-reference nucleotide and/or amino acid sequences which are present in the file named “239_PCT1_ST25.txt”, which is 46 kilobytes in size, and which was created Nov. 8, 2012 in the IBM-PCT machine format, having an operating system compatibility with MS-Windows.

### FIELD OF THE INVENTION

Provided herein are compositions and methods for treating a subject at risk of or afflicted with a disease, a disorder or an injury, particularly a disease, a disorder or an injury of the nervous system.

### BACKGROUND OF THE INVENTION

U.S. Pat. Nos. 6,740,738; 7,741,299 and 7,872,119 disclose RTP801 inhibitors.

U.S. Patent Application Publication Nos., US 2011/0028532; US 2011/0117102; US 2010/0272722 and US 2011/0028531 disclose dsRNA RTP801 molecules.

U.S. Pat. No. 7,626,015 and US Patent Application Publication No. 20110105584 disclose dsRNA RTP801L (REDD2) molecules.

US Patent Application Publication Nos. 20090162365 and 20110112168 disclose dsRNA Casp2 and RhoA molecules.

U.S. Patent Application Publication No. US2009/0162365 and PCT Patent Publication No. WO 2009/044392 are directed to therapeutics useful in treating certain ocular and neurological diseases and disorders.

PCT Publication Nos. WO 2006/023544 and WO 2010/048352 are directed to compositions and methods useful in treating ocular diseases.

An effective treatment to promote neuroprotection and/or axonal growth is desired. It is, accordingly, an aspect to provide methods for promoting neuroprotection, axonal outgrowth and neural progenitor cell stimulation utilizing compounds not previously known to have these activities.

### SUMMARY OF THE INVENTION

This disclosure is directed to methods of treating a subject afflicted with a disease, disorder or injury of a nervous system. In some embodiments the methods comprise administering to the subject two therapeutic agents, which down regulate, or target the expression of at least two genes associated with the nervous system disorder or injury. In one embodiment the first agent targets a gene selected from a pro-apoptotic gene and the second agent targets the RTP801 gene (also known as REDD1 and DDIT4) or the REDD2 gene (also known as RTP801L and DDIT4L). In one embodiment, the first agent targets Caspase2 (Casp2) and the second agent targets RTP801. In another embodiment one agent targets Casp2 and the second agent targets REDD2. An agent that down regulates expression of a target gene may be referred to as an inhibitor. In some embodiments the method comprises administering to the subject a Casp2 inhibitor and a RTP801 inhibitor. In some embodiments the proapoptotic gene comprises RhoA. In some embodiments the method comprises administering to the subject a RhoA inhibitor and a RTP801 inhibitor. In some embodiments at least one agent comprises a nucleic acid molecule. In some embodiments the first agent and the second agent comprise oligonucleotide molecules. In some embodiments, the nervous system relates to the central nervous system (CNS) and/or the peripheral nervous system (PNS). In another aspect, RTP801 inhibitors, such as double-stranded RNA (dsRNA) agents that down regulate RTP801 expression/biological activity are now disclosed as useful in promoting neurite outgrowth, axonal regeneration or neural regeneration and stimulating proliferation and/or differentiation of neural progenitor cells.

In one aspect, provided herein is a therapeutic combination comprising a RTP801 inhibitor or a REDD2 inhibitor; and a Casp2 inhibitor for use in treating a subject suffering from, or at risk of, developing a disease. In some embodiments, each of the RTP801 inhibitor or the REDD2 inhibitor; and the Casp2 inhibitor is independently selected from the group consisting of an antibody, a polypeptide, a peptide, a nucleic acid molecule and a small organic molecule. In preferred embodiments, each of the RTP801 inhibitor or the REDD2 inhibitor; and the Casp2 inhibitor is independently a nucleic acid molecule, preferably a double-stranded RNA (dsRNA) compound comprising an antisense strand and a sense strand. In some embodiments the RTP801 inhibitor comprises a RTP801 double-stranded RNA compound, wherein the antisense strand comprises the sequence:

(SEQ ID NO: 7 or 9)
5′ AGCUGCAUCAGGUUGGCAC 3′.

In some embodiments the REDD2 inhibitor comprises a REDD2 double-stranded RNA compound. In some embodiments, the therapeutic combination includes a Casp2 double-stranded RNA, preferably the Casp2 dsRNA compound comprises the antisense sequence:

(SEQ ID NO: 11 or 13)
5′ AGGAGUUCCACAUUCUGGC 3′.

In preferred embodiments the therapeutic combination includes a RTP801 double-stranded RNA compound having the structure:

(antisense strand, SEQ ID NO: 7)
5′ AGCUGCAUCAGGUUGGCAC 3′
(sense strand; SEQ ID NO: 8)
3′ UCGACGUAGUCCAACCGUG 5′

wherein the ribonucleotide at the 5′ terminus and the ribonucleotide at the 3′ terminus of the antisense strand is a 2′-O-methyl sugar modified ribonucleotide;
wherein the ribonucleotide at the 5′ terminus and the ribonucleotide at the 3′ terminus of the sense strand is an unmodified ribonucleotide;
wherein the remaining ribonucleotides in the antisense strand and in the sense strand comprise alternating unmodified ribonucleotides and 2′-O-methyl sugar modified ribonucleotides;
wherein the ribonucleotide at each of the 5′ terminus and the 3′ terminus of the antisense strand and the sense strand is independently phosphorylated or non-phosphorylated, preferably the 3′ terminus of the antisense strand and of the sense strand are phosphorylated (SEQ ID NO:9) and (SEQ ID NO:10); and
wherein the Casp2 double-stranded RNA compound has the structure:

(antisense strand, SEQ ID NO: 11)
5′ AGGAGUUCCACAUUCUGGC 3′
(sense strand, SEQ ID NO: 12)
3′ UCCUCAAGGUGUAAGACCG-iB 5′

wherein each A, C, U and G is joined to the next A, C, U, and G by a covalent bond;
wherein the sense strand comprises, counting from the 5′ terminus, an unmodified ribonucleotide at positions 1, 2, 3, 4, 5, 6, 7, 8, 9, 10, 11, 12, 13, 14, 15, 16, 17 and 19, a L-deoxycytidine at position 18, and an inverted abasic 5′ cap; and
wherein the antisense strand comprises, counting from the 5′ terminus, 2′-O-methyl sugar modified ribonucleotide at positions 2, 4, 6, 8, 11, 13, 15, 17 and 19 and unmodified ribonucleotide at positions 1, 3, 5, 7, 9, 10, 12, 14, 16 and 18.

Preferred dsRNA compounds targeting RTP801 include an antisense sequence selected from any one of SEQ ID NO:7, 15, 17, 19, 21, 23, 25, 27 or 29.

In preferred embodiments, the covalent bond joining each A, C, U and G to the next A, C, U and G in the RTP801 dsRNA and in the Casp2 dsRNA is a phosphodiester bond. In some embodiments, the disease comprises neurodegeneration or is a disease associated with a physically damaged nerve and/or neurite damage. In various embodiments the disease is selected from the group consisting of an ocular disease, an ocular disorder and an ocular injury, preferably the an ocular injury selected from the group consisting of ischemic injury, ischemia-reperfusion injury, mechanical injury, and injury or interruption of nerve fibers, and/or is associated with lack of retrograde supply of neurotrophic factor. In some embodiments the disease is selected from the group consisting of physical damage to the central and/or peripheral nervous system; brain damage associated with stroke, amyotrophic lateral sclerosis (ALS), multiple sclerosis (MS), progressive muscular atrophy, Gullain-Barre syndrome, Alzheimer's disease, Huntington's Disease, Parkinson's disease, episodic vertigo, hearing loss, tinnitus and aural fullness, diabetic neuropathy, increased intraocular pressure, open angle glaucoma, angle closure glaucoma, diabetic retinopathy (DR), diabetic macular edema (DME), age related macular degeneration (AMD), Leber's hereditary optic neuropathy (LHON), Leber's optic atrophy, optic neuritis, retinal artery occlusion, central retinal vein occlusion, branch retinal vein occlusion, ischemic optic neuropathy including non-arteritic ischemic optic neuropathy (NAION), optic nerve injury, retinopathy of prematurity (ROP) or retinitis pigmentosa (RP), retinal ganglion degeneration, macular degeneration, hereditary optic neuropathy, metabolic optic neuropathy, optic neuropathy due to a toxic agent and neuropathy caused by adverse drug reactions or a vitamin deficiency.

In some embodiments of the therapeutic combination the RTP801 inhibitor is configured for simultaneous administration with the Casp2 inhibitor. In some embodiments of the therapeutic combination the REDD2 inhibitor is configured for simultaneous administration with the Casp2 inhibitor. Alternatively, the RTP801 inhibitor is configured for administration prior to or subsequently to administration of the Casp2 inhibitor. In some embodiments the REDD2 inhibitor is configured for administration prior to or subsequently to administration of the Casp2 inhibitor. Each of the inhibitors of the therapeutic combination is configured for administration in the same or in different doses, for example in a ratio from about 1:1000 to 1000:1 (ug/eye) RTP801 inhibitor:Casp2 inhibitor. In some embodiments, when the RTP801 inhibitor is a double-stranded RNA compound, e.g. SEQ ID NO:7 and 8, and the Casp2 inhibitor is a double-stranded RNA compound, e.g. SEQ ID NO:13 and 14; the RTP801 inhibitor and the Casp2 inhibitor are configured for administration in a ratio from about 1:1 to 1000:1 RTP801 inhibitor:Casp2 inhibitor, or in a ratio from about 1:10 to 1000:1 RTP801 inhibitor:Casp2 inhibitor. In some embodiments the administration is invasive, i.e. intravitreal injection, or non-invasive, i.e. eye drops or ointment.

The therapeutic combination comprising a RTP801 inhibitor or a REDD2 inhibitor; and a Casp2 inhibitor is further provided for use in providing neuroprotection to a neuron in a subject in need thereof. In some embodiments, the neuron is, or is comprised within, a system selected from the group consisting of a peripheral nervous system, a central nervous system and an audio-vestibular system. in particular a visual system of a central nervous system. In some embodiments the neuron is a ganglion cell and the ganglion cell is selected from the group consisting of a retinal ganglion cell, a spiral ganglion cell, a vestibular ganglion cell, a dorsal ganglion cell and a peripheral ganglion cell. In some embodiments, the neuroprotection comprises protecting the neuron from death, for example apoptotic cell death. The death of the neuron is associated with one or more of a disease or disorder, a surgery, ischemia, ischemia/reperfusion, physical/mechanical trauma, a chemical agent, an infectious agent, an immunologic reaction and a nutritional imbalance.

In a second aspect, disclosed herein is a composition which includes an RTP801 inhibitor or a REDD2 inhibitor; and a Casp2 inhibitor, and a pharmaceutically acceptable carrier. In some embodiments, each of the RTP801 inhibitor or the REDD2 inhibitor; and the Casp2 inhibitor is independently selected from the group consisting of an antibody, a polypeptide, a peptide, a nucleic acid molecule and a small organic molecule. In preferred embodiments, each of the RTP801 inhibitor or the REDD2 inhibitor; and the Casp2 inhibitor is independently a nucleic acid molecule, preferably a double-stranded RNA (dsRNA) compound comprising an antisense strand and a sense strand. In some embodiments the RTP801 inhibitor comprises a RTP801 double-stranded RNA compound, wherein the antisense strand comprises the sequence:

(SEQ ID NO: 7)
5′ AGCUGCAUCAGGUUGGCAC 3′.

In some embodiments the REDD2 inhibitor comprises a REDD2 double-stranded RNA compound. In some embodiments, the composition includes a Casp2 double-stranded RNA, preferably the Casp2 dsRNA compound comprises the antisense sequence:

(SEQ ID NO: 11)
5′ AGGAGUUCCACAUUCUGGC 3'.

In preferred embodiments the composition includes a RTP801 double-stranded RNA compound having the structure:

(antisense strand, SEQ ID NO: 7)
5′ AGCUGCAUCAGGUUGGCAC 3′
(sense strand; SEQ ID NO: 8)
3′ UCGACGUAGUCCAACCGUG 5′

wherein the ribonucleotide at the 5′ terminus and the ribonucleotide at the 3′ terminus of the antisense strand is a 2′-O-methyl sugar modified ribonucleotide;
wherein the ribonucleotide at the 5′ terminus and the ribonucleotide at the 3′ terminus of the sense strand is an unmodified ribonucleotide;
wherein the remaining ribonucleotides in the antisense strand and in the sense strand comprise alternating unmodified ribonucleotides and 2′-O-methyl sugar modified ribonucleotides;
wherein the ribonucleotide at each of the 5′ terminus and the 3′ terminus of the antisense strand and the sense strand is independently phosphorylated or non-phosphorylated, preferably the 3′ terminus of the antisense strand and of the sense strand are phosphorylated (SEQ ID NO:9) and (SEQ ID NO:10); and
wherein the Casp2 double-stranded RNA compound has the structure:

(antisense strand, SEQ ID NO: 11)
5′ AGGAGUUCCACAUUCUGGC 3′
(sense strand, SEQ ID NO: 12)
3′ UCCUCAAGGUGUAAGACCG-iB 5′

wherein each A, C, U and G is joined to the next A, C, U, and G by a covalent bond;
wherein the sense strand comprises, counting from the 5′ terminus, an unmodified ribonucleotide at positions 1, 2, 3, 4, 5, 6, 7, 8, 9, 10, 11, 12, 13, 14, 15, 16, 17 and 19, a L-deoxycytidine at position 18, and an inverted abasic 5′ cap; and
wherein the antisense strand comprises, counting from the 5′ terminus, 2′-O-methyl sugar modified ribonucleotide at positions 2, 4, 6, 8, 11, 13, 15, 17 and 19 and unmodified ribonucleotide at positions 1, 3, 5, 7, 9, 10, 12, 14, 16 and 18.

In preferred embodiments, the covalent bond joining each A, C, U and G to the next A, C, U and G in the RTP801 dsRNA and in the Casp2 dsRNA is a phosphodiester bond. Each of the inhibitors in the composition is present in a ratio from about 1:1 to 1000:1 RTP801 inhibitor:Casp2 inhibitor. In some embodiments, when the RTP801 inhibitor is a double-stranded RNA compound, e.g. SEQ ID NO:7 and 8, and the Casp2 inhibitor is a double-stranded RNA compound, e.g. SEQ ID NO:13 and 14; the RTP801 inhibitor and the Casp2 inhibitor are configured for administration present in a ratio from about 10:1 to 1000:1 RTP801 inhibitor:Casp2 inhibitor. The composition is useful in the treatment of a subject suffering from or at risk of developing a disease, a disorder or an injury, for example a disease, disorder, or injury associated with a physically damaged nerve and/or neurite damage. In some embodiments the composition is useful in the treatment of a subject suffering from or at risk of developing an ocular disease, an ocular disorder and an ocular injury, including for example, ischemic injury, ischemia-reperfusion injury, mechanical injury, injury or interruption of nerve fibers and/or is associated with lack of retrograde supply of neurotrophic factor. In other embodiments the disease is selected from the group of diseases and disorders described hereinabove and infra.

In another aspect, provided herein is an inhibitor selected from the group consisting of a RTP801 inhibitor or a salt thereof and a REDD2 inhibitor or a salt thereof, for use in promoting neurite outgrowth, axonal regeneration and/or neural regeneration, wherein the inhibitor is configured for contacting a neuron. In some embodiments, the RTP801 inhibitor or a salt thereof and a REDD2 inhibitor or a salt thereof, is useful in promoting neurite outgrowth, axonal regeneration and/or neural regeneration in a subject in need thereof or in maintaining the viability of a neuron in a peripheral nervous system and/or a central nervous system, including a visual system, and/or an audio-vestibular system. In other embodiments the inhibitor is useful in preventing, treating, or reducing symptoms of nerve injury in a subject. The RTP801 inhibitor or the REDD2 inhibitor is selected from the group consisting of an antibody, a polypeptide, a peptide, a nucleic acid molecule and a small organic molecule. In preferred embodiments the inhibitor is a nucleic acid molecule, preferably a chemically modified dsRNA which includes a sense strand and an antisense strand. In some embodiments the RTP801 dsRNA includes a antisense strand having the sequence:

(SEQ ID NO: 7)
5′ AGCUGCAUCAGGUUGGCAC 3′.

In preferred embodiments, the RTP801 dsRNA compound has the structure:

5′ AGCUGCAUCAGGUUGGCAC 3′ (antisense strand)
3′ UCGACGUAGUCCAACCGUG 5′ (sense strand)

wherein the ribonucleotide at the 5′ terminus and the ribonucleotide at the 3′ terminus of the antisense strand is a 2′-O-methyl modified ribonucleotide;
wherein the ribonucleotide at the 5′ terminus and the ribonucleotide at the 3′ terminus of the sense strand is an unmodified ribonucleotide;
wherein remaining ribonucleotides in the antisense strand and in the sense strand comprise alternating unmodified ribonucleotides and 2′-O-methyl sugar modified ribonucleotides; and
wherein the ribonucleotides at each of the 5′ terminus and the 3′ terminus of the antisense strand and the sense strand are independently phosphorylated or non-phosphorylated. Preferably each of the 3′ terminus of the antisense strand and of the sense strand are phosphorylated (SEQ ID NO:7 and 8).

In some embodiments, the neuron or nerve is, or is comprised within a system selected from a peripheral nervous system and a central nervous system, and an audio-vestibular system, preferably the visual system of a central nervous system. In some embodiments the neuron is a ganglion cell for example, a ganglion cell selected from the group consisting of a retinal ganglion cell, a spiral ganglion cell, a vestibular ganglion cell, dorsal root ganglion and a peripheral ganglion cell. In various embodiments, the neuron is derived from a stem cell or from a progenitor cell, for example a stem cell known as Muller's glia.

In another aspect, provided herein is a RTP801 inhibitor or a REDD2 inhibitor for use in the treatment of a disease or condition benefiting from promotion of neuronal growth and/or repair, for example in promoting neurite outgrowth, axonal regeneration or neural regeneration. In some embodiments the promoting neurite outgrowth, axonal regeneration or neural regeneration occurs within the optic nerve.

Further provided herein is a kit comprising a RTP801 inhibitor or a REDD2 inhibitor; and a Casp2 inhibitor; and instructions for use. In some embodiments the use is for treatment of a disease, disorder, or injury comprising neurodegeneration and/or associated with a physically damaged nerve and/or neurite damage. In some embodiments the use is for treatment of an ocular disease, an ocular disorder or an ocular injury, for example wherein the ocular injury includes ischemic injury, ischemia-reperfusion injury, mechanical injury, injury or interruption of nerve fibers and/or is associated with lack of supply of neurotrophic factor. In various embodiments of the kit, the disease is selected from the group of diseases and disorders described hereinabove and infra.

In yet another aspect, provided herein is a method of treating a subject suffering from or at risk of developing a disease which comprises administering to the subject a therapeutically effective amount of a RTP801 inhibitor or a REDD2 inhibitor; and a therapeutically effective amount of a Casp2 inhibitor, so as to thereby treat the subject. Further provided is the use of a composition comprising a RTP801 inhibitor or a REDD2 inhibitor; and a Casp2 inhibitor in the manufacture of a medicament for treating a subject suffering from or at risk of developing a disease.

In some embodiments of the method or use, each of the RTP801 inhibitor or the REDD2 inhibitor; and the Casp2 inhibitor is independently selected from the group consisting of an antibody, a polypeptide, a peptide, a nucleic acid molecule and a small organic molecule, preferably each inhibitor is a nucleic acid molecule. In preferred embodiments each nucleic acid molecule comprises a double-stranded RNA (dsRNA) compound comprising an antisense strand and a sense strand. In various embodiments of the method or use, the RTP801 inhibitor comprises a RTP801 double-stranded RNA compound, wherein the antisense strand includes the sequence:

(SEQ ID NO: 7 or 9)
5′ AGCUGCAUCAGGUUGGCAC 3′.

In some embodiments of the method or use the REDD2 inhibitor comprises a REDD2 double-stranded RNA compound. In some embodiments of the method or use antisense strand of the Casp2 double-stranded RNA compound comprises the sequence:

(SEQ ID NO: 11 or 13)
5′ AGGAGUUCCACAUUCUGGC 3′.

In preferred embodiments of the method or use, the RTP801 double-stranded RNA compound has the structure:

(antisense strand, SEQ ID NO: 7)
5′ AGCUGCAUCAGGUUGGCAC 3′
(sense strand; SEQ ID NO: 8)
3′ UCGACGUAGUCCAACCGUG 5′

wherein the ribonucleotide at the 5′ terminus and the ribonucleotide at the 3′ terminus of the antisense strand is a 2′-O-methyl sugar modified ribonucleotide;
wherein the ribonucleotide at the 5′ terminus and the ribonucleotide at the 3′ terminus of the sense strand is an unmodified ribonucleotide;
wherein the remaining ribonucleotides in the antisense strand and in the sense strand comprise alternating unmodified ribonucleotides and 2′-O-methyl sugar modified ribonucleotides;
wherein the ribonucleotide at each of the 5′ terminus and the 3′ terminus of the antisense strand and the sense strand is independently phosphorylated or non-phosphorylated, preferably the 3′ terminus of the antisense strand and of the sense strand are phosphorylated (SEQ ID NO:9) and (SEQ ID NO:10); and
the Casp2 double-stranded RNA compound has the structure:

(antisense strand, SEQ ID NO: 11)
5′ AGGAGUUCCACAUUCUGGC 3′
(sense strand, SEQ ID NO: 12)
3′ UCCUCAAGGUGUAAGACCG-iB 5′

wherein each A, C, U and G is joined to the next A, C, U, and G by a covalent bond;
wherein the sense strand comprises, counting from the 5′ terminus, an unmodified ribonucleotide at positions 1, 2, 3, 4, 5, 6, 7, 8, 9, 10, 11, 12, 13, 14, 15, 16, 17 and 19, a L-deoxycytidine at position 18, and an inverted abasic 5′ cap; and
wherein the antisense strand comprises, counting from the 5′ terminus, 2′-O-methyl sugar modified ribonucleotide at positions 2, 4, 6, 8, 11, 13, 15, 17 and 19 and unmodified ribonucleotide at positions 1, 3, 5, 7, 9, 10, 12, 14, 16 and 18.

In preferred embodiments of the method or use, the covalent bond joining each A, C, U and G to the next A, C, U and G in the RTP801 dsRNA and in the Casp2 dsRNA is a phosphodiester bond. In some embodiments the method or use, the disease comprises neurodegeneration or is a disease associated with a physically damaged nerve and/or neurite damage. In preferred embodiments, the disease is selected from the group of diseases and disorders described hereinabove and infra.

In some embodiments of the method or use, the RTP801 inhibitor is configured for simultaneous administration with the Casp2 inhibitor. In some embodiments of the of the method or use the REDD2 inhibitor is configured for simultaneous administration with the Casp2 inhibitor. Alternatively, the RTP801 inhibitor is configured for administration prior to or subsequently to administration of the Casp2 inhibitor. In some embodiments the REDD2 inhibitor is configured for administration prior to or subsequently to administration of the Casp2 inhibitor. Each of the inhibitors is configured for administration in the same or in different doses, for example in a ratio from about 1:1 to 1000:1 RTP801 inhibitor:Casp2 inhibitor. In some embodiments, when the RTP801 inhibitor is a double-stranded RNA compound, e.g. SEQ ID NO:7 and 8, and the Casp2 inhibitor is a double-stranded RNA compound, e.g. SEQ ID NO:13 and 14; the RTP801 inhibitor and the Casp2 inhibitor are configured for administration in a ratio from about 10:1 to 1000:1 RTP801 inhibitor:Casp2 inhibitor. Administration methods encompass invasive and non-invasive methods.

The methods and use comprising a RTP801 inhibitor or a REDD2 inhibitor; and a Casp2 inhibitor are further provided for neuroprotection to a neuron in a subject in need thereof. In some embodiments, the neuron is, or is comprised within, a system selected from the group consisting of a peripheral nervous system, a central nervous system and an audio-vestibular system. in particular a visual system of a central nervous system. In some embodiments the neuron is a ganglion cell and the ganglion cell is selected from the group consisting of a retinal ganglion cell, a spiral ganglion cell, a vestibular ganglion cell, a dorsal ganglion cell and a peripheral ganglion cell. In some embodiments, the neuroprotection comprises protecting the neuron from death, for example apoptotic cell death. The death of the neuron is associated with one or more of a disease or disorder, a surgery, ischemia, ischemia/reperfusion, physical/mechanical trauma, a chemical agent, an infectious agent, an immunologic reaction and a nutritional imbalance.

In various embodiments of the method or use, the disease is selected the disease is selected from the group of diseases and disorders described hereinabove and infra.

In some embodiments of the method or use, the RTP801 inhibitor is administered simultaneously with the Casp2 inhibitor. In some embodiments of the of the method or use, the REDD2 inhibitor is administered simultaneously with the Casp2 inhibitor. Alternatively, the RTP801 inhibitor is administered prior to or subsequently to administration of the Casp2 inhibitor. In some embodiments the REDD2 inhibitor is administered prior to or subsequently to administration of the Casp2 inhibitor. Each of the inhibitors of the method or use is administered in the same or in different doses, for example in a ratio from about 1:1 to 1000:1 RTP801 inhibitor:Casp2 inhibitor or REDD2 inhibitor:Casp2 inhibitor. In some embodiments, when the RTP801 inhibitor is a double-stranded RNA compound, e.g. SEQ ID NO:7 and 8, and the Casp2 inhibitor is a double-stranded RNA compound, e.g. SEQ ID NO:13 and 14; the RTP801 inhibitor and the Casp2 inhibitor are administered in a ratio from about 10:1 to 1000:1 RTP801 inhibitor:Casp2 inhibitor.

The methods and use disclosed herein provide neuroprotection to a neuron in a subject in need thereof, comprising administering to the subject an RTP801 inhibitor or a REDD2 inhibitor; and a Casp2 inhibitor; so as to thereby provide neuroprotection to the neuron in the subject. Further provided herein is the use of an RTP801 inhibitor or a REDD2 inhibitor; and a Casp2 inhibitor in the manufacture of a medicament for providing neuroprotection to a neuron in a subject in need thereof. In some embodiments, the neuron is, or is comprised within, a system selected from the group consisting of a peripheral nervous system, a central nervous system, and an audio-vestibular system, preferably the visual system of a central nervous system. In some embodiments, the neuron is a ganglion cell, for example, a ganglion cell selected from the group consisting of a retinal ganglion cell, a spiral ganglion cell, a vestibular ganglion cell, dorsal root ganglion and a peripheral ganglion cell. In some embodiments, the neuroprotection comprises protecting the neuron from death, for example apoptotic cell death. Death of the neuron is associated with one or more of a disease or disorder, a surgery, ischemia, ischemia/reperfusion, physical/mechanical trauma, a chemical agent, an infectious agent, an immunologic reaction and a nutritional imbalance.

In another aspect, described herein is a method of promoting neurite outgrowth, axonal regeneration or neural regeneration comprising contacting a neuron with an effective amount of an RTP801 inhibitor or a salt thereof or of a REDD2 inhibitor or a salt thereof, thereby promoting neurite outgrowth, axonal regeneration or neural regeneration. Further described is a method of promoting neurite outgrowth, axonal regeneration or neural regeneration in a subject in need thereof, comprising administering to the subject a therapeutically effective amount of a RTP801 inhibitor or a pharmaceutically acceptable salt thereof; or a therapeutically effective amount of a REDD2 inhibitor or a pharmaceutically acceptable salt thereof, thereby promoting neurite outgrowth, axonal regeneration or neural regeneration in the subject. In another aspect, provided is a method for maintaining the viability of a neuron in a peripheral nervous system and/or a central nervous system, including a visual system, and/or an audio-vestibular system, comprising contacting the neuron with an RTP801 inhibitor or with a REDD2 inhibitor, thereby maintaining the viability of a neuron in the central nervous system, the visual system and/or the vestibular system. In yet another aspect, provided is a method of preventing, treating, or reducing symptoms of nerve injury in a subject, wherein the method comprises administering to the subject an effective amount of an agent that reduces the expression or activity of RTP801 or of REDD2, to prevent, treat, or reduce symptoms of nerve injury. In another aspect, the disclosure relates to a method of treating a subject suffering nerve damage comprising the step of administering a composition comprising an RTP801 inhibitor or a REDD2 inhibitor to the subject, thereby treating the nerve damage in the subject. In yet another aspect, the disclosure provides the use of an RTP801 inhibitor or a salt thereof or of a REDD2 inhibitor or a salt thereof in the manufacture of a medicament for promoting neurite outgrowth, axonal regeneration or neural regeneration and the use of an RTP801 inhibitor or a REDD2 inhibitor in the manufacture of a medicament for maintaining the viability of a neuron in a peripheral nervous system and/or a central nervous system, including a visual system, and/or an audio-vestibular system. Further provided is the use of an RTP801 inhibitor or a REDD2 inhibitor in the manufacture of a medicament for preventing, treating, or reducing symptoms of nerve injury in a subject or for the manufacture of a medicament for treating a subject suffering nerve damage. In some embodiments of the methods and use described herein the RTP801 inhibitor or the REDD2 inhibitor is selected from the group consisting of an antibody, a polypeptide, a peptide, a nucleic acid molecule and a small organic molecule. In some embodiments the RTP801 inhibitor or the REDD2 inhibitor is a nucleic acid molecule, preferably a dsRNA comprising an antisense strand and a sense strand. In preferred embodiments the antisense strand of the RTP801 inhibitor double-stranded RNA compound comprises the sequence:

(SEQ ID NO: 7 or 9)
5′ AGCUGCAUCAGGUUGGCAC 3′

and the RTP801 double-stranded RNA compound has the structure:

(antisense strand; SEQ ID NO: 7)
5′ AGCUGCAUCAGGUUGGCAC 3′
(sense strand; SEQ ID NO: 8)
3′ UCGACGUAGUCCAACCGUG 5′

wherein the ribonucleotide at the 5′ terminus and the ribonucleotide at the 3′ terminus of the antisense strand is a 2′-O-methyl modified ribonucleotide;
wherein the ribonucleotide at the 5′ terminus and the ribonucleotide at the 3′ terminus of the sense strand is an unmodified ribonucleotide;
wherein remaining ribonucleotides in the antisense strand and in the sense strand comprise alternating unmodified ribonucleotides and 2′-O-methyl sugar modified ribonucleotides; and
wherein the ribonucleotides at each of the 5′ terminus and the 3′ terminus of the antisense strand and the sense strand are independently phosphorylated or non-phosphorylated. According to the method or use of described herein, the neuron is, or is comprised within a system selected from a peripheral nervous system and a central nervous system, and an audio-vestibular system, preferably the visual system of a central nervous system. In some embodiments, the neuron is a ganglion cell, for example a retinal ganglion cell, a spiral ganglion cell, a vestibular ganglion cell, dorsal root ganglion and a peripheral ganglion cell. In various embodiments, the neuron is derived from a stem cell or from a progenitor cell, for example a stem cell known as Muller's glia.

In preferred embodiments of the therapeutic combinations, compositions, methods and uses, the double stranded RNA compound is chemically modified according to the embodiments of disclosed herein and/or embodiments presented in PCT publication Nos. WO 2006/023544, WO 2010/048352, WO2009/116037, WO 2009/147684, WO 2011/066475 and WO 2011/084193, all assigned to the assignee of the instant application.

The methods, materials, and examples that will now be described are illustrative only and are not intended to be limiting; materials and methods similar or equivalent to those described herein can be used in practice or testing of the invention. Other features and advantages of the invention will be apparent from the following detailed description, and from the claims.

This disclosure is intended to cover any and all adaptations or variations of combination of features that are disclosed in the various embodiments herein. Although specific embodiments have been illustrated and described herein, it should be appreciated that the invention encompasses any arrangement of the features of these embodiments to achieve the same purpose. Combinations of the above features, to form embodiments not specifically described herein, will be apparent to those of skill in the art upon reviewing the instant description.

### BRIEF DESCRIPTION OF THE FIGURES

FIG. 1 provides data for mean quantity of retinal ganglion cells (RGC) per 250 um of central retina in intact and dsRNA-treated eyes in a rat optic nerve crush (ONC) model.

FIG. 2 presents data representing quantification of RGC axon outgrowth in dsRNA-treated eyes at 24 days after optic nerve crush (ONC).

FIG. 3 provides evidence that inhibition of RTP801 expression by dsRTP801 in mouse eyes induces expression of PEDF and thrombospondin.

FIG. 4 shows the experimental procedure in the rat model of optic nerve crush used to assess neuroprotective and neuroregenerative effects of dsRTP801.

FIG. 5 shows level of neuroprotection in dsRTP801-injected eyes at Day 24 after ONC. For group identified in FIG. 5′ X-axes legend as “siEGFP”, the treatment was with 40 ug/eye of “siEGFP”. For group identified in FIG. 5′ X-axes legend as “PF-655”, the treatment was with 20 ug/eye of “PF-655”+20 ug/eye of “siEGFP”. For RGC counts, 20×15 μm thick sections of the eye, with the optic nerve clearly visible, were taken and every 4th section was selected and stained for β-III tubulin and DAPI to reveal the RGC. A linear 250 μm region of the retina either side of the optic nerve was used to count numbers of β-III+RGC/250 μm of retina.

FIG. 6 shows enhanced axonal growth in dsRTP801-treated rat eyes at day 24 following ONC. For group identified in FIG. 6′ X-axes legend as “siEGFP”, the treatment was with 40 ug/eye of “siEGFP”. For group identified in FIG. 6′ X-axes legend as “PF-655”, the treatment was with 20 ug/eye of “PF-655”+20 ug/eye of “siEGFP”. The number of GAP43-positive axons crossing a line drawn at fixed distances from the center of the lesion site was counted in 3 longitudinal sections of the optic nerve in each eye. The cross-sectional width of the optic nerve, measured at the point at which the counts were taken, was used to calculate the number of axons/mm width, and this was averaged over the 3 sections. Σad, the total number of axons extending distance d in a nerve having a radius of r, was estimated by summing over all sections having a thickness t (15 μm): Σad=pr2×(average number of axons/mm width)/t.

FIG. 7 presents fluorescent histological sections of neurons showing axon outgrowth induced by “PF-655” in the rat ONC model.

FIG. 8 shows a lack of interferon response following IVT injection of “PF-655” in rats. “PF-655” is in about a 30 fold molar excess compared to Poly(I:C). FIG. 8 shows that axonal regeneration is specific to RTP801 inhibition and is not related to inflammatory response or innate immune response.

FIGS. 9-11 show the effect of “PF-655” on stem phenotype of Muller glia (MG) cells. Successful regeneration of retinal cells (ganglion, photoreceptors, astroglia) is at least partially dependent on Muller glia (MG) that respond to retinal injury by generating multipotent progenitors that can regenerate all major retinal cell types. Attempts to stimulate MG dedifferentiation and retinal regeneration in mammals have met with little success until now. Layers are labeled as follows: INL: inner nuclear layer; IPL: inner plexiform layer: GCL: ganglion cell layer. SOX2 staining in the INL indicated total number of MG cells; GFAP in the IPL indicated activated MG cells (=increased GFAP expression); Sox2 in the IPL indicated cells with elevated Sox2 expression (=elevated progenitor competence); Nestin in the IPL indicated elevated expression of Nestin (=activation of a more stem phenotype).

FIG. 12 shows that intact eyes intravitreally injected naked dsRNAs reach different cell layers in rat retina, as viewed by in situ hybridization with a ³³P end labeled sense strand probe. Layers are labeled as follows: RGC: retinal ganglion cells; INL: inner nuclear layer; ONL: outer nuclear layer; RPE: retinal pigment epithelium.

FIG. 13 shows that RGC dendritic arbors retract soon after axotomy and prior to cell death in RGC-YFP Transgenic Mice Model.

FIG. 14 shows that mTOR activity is down regulated in injured RGCs in RGC-YFP Transgenic Mice Model.

FIG. 15 shows that down-regulation of REDD2 protects RGC dendrites in RGC-YFP Transgenic Mice Model.

FIGS. 16A and 16B show that down-regulation of REDD2 (“Axo+siREDD2” row in FIG. 16A; “Axo+siREDD2” bar in FIG. 16B) prevents loss of excitatory inputs onto RGCs in RGC-YFP Transgenic Mice Model.

FIG. 17 shows results obtained in RGC-YFP Transgenic Mice Model. Total dendritic length is slightly increased as a result of down-regulation of DDIT4 (“Axo+siDDIT2/siScram” bar), as well as with down-regulation of REDD2 (“Axo+siREDD2/siScram” bar), compared to a negative control (“Axo+siScram” bar). Total dendritic length is further increased as a result of down-regulation of Casp2 (“Axo+siCasp2/siScram” bar), compared to a negative control (“Axo+siScram” bar). In addition, there is a further slight increase in total dendritic length if dsRNA targeting Casp2 (siCasp2) is combined with a dsRNA targeting RTP801 (DDIT4) (“Axo+siCasp2/siDDIT4” bar)

FIG. 18 shows results obtained in RGC-YFP Transgenic Mice Model. Treatment with a RTP801 inhibitor (“Axo+siDDIT4/siScram” bar), reduces dendritic field area, compared to control dsRNA (“Axo+siScram” bar). A more prominent reduction of dendritic field area is achieved with a REDD2 inhibitor (“Axo+REDD2/siScram” bar), compared to control dsRNA (“Axo+siScram” bar). Down-regulation of Casp2 (“Axo+siCasp2/siScram” bar) increases dendritic field area, compared to control dsRNA (“Axo+siScram” bar). Combined treatment with a dsRNA targeting Casp2 (siCasp2) and a dsRNA targeting RTP801 (DDIT4) (“Axo+siCasp2/siDDIT4” bar) leaves the area unchanged compared to control dsRNA (“Axo+siScram” bar)

FIG. 19 shows results obtained in RGC-YFP Transgenic Mice Model. Treatment with a RTP801 inhibitor (“Axo+siDDIT4/siScram” bar) results in a significant increase of the total number of dendritic branches per neuron, compared to control (“Axo+siScram” bar). Significantly greater effect (increase of the total number of dendritic branches per neuron) is obtained with REDD2 inhibitor monotherapy (“Axo+siREDD2/siScram” bar). Down-regulation of Casp2 (“Axo+siCasp2/siScram” bar) has no effect on the total number of dendritic branches per neuron compared to control dsRNA (“Axo+siScram” bar). The total number of dendritic branches per neuron obtained with combined treatment: dsRNA targeting Casp2 (siCasp2) and a dsRNA targeting siRTP801 (siDDIT4) (“Axo+siCasp2/siDDIT4” bar), had a similar effect on total number of dendritic branches per neuron, to the one obtained with siRTP801 monotherapy.

FIG. 20 shows results obtained in RGC-YFP Transgenic Mice Model. Treatment with a RTP801 inhibitor (“Axo+siDDIT4/siScram” bar) results in a significant increase of the total number of terminal branches per neuron. Down-regulation of Casp2 (“Axo+siCasp2/siScram” bar) has no effect on the total number of terminal branches per neuron compared to control dsRNA (“Axo+siScram” bar). There is a further slight increase of the total number of terminal branches per neuron if dsRNA targeting Casp2 (siCasp2) is combined with a dsRNA targeting si801 (siDDIT4) (“Axo+siCasp2/siDDIT4” bar).

FIG. 21 shows results obtained in RGC-YFP Transgenic Mice Model. Down-regulation of Casp2 (“QPI1007”) results in longer dendrites, whereas treatment with a RTP801 inhibitor (“Axo+siDDIT4/siScram” bar) results in branching.

FIG. 22 shows the experimental outline of experiments performed using Oxygen-Induced Retinopathy (OIR) model system for evaluation of protection of Retinal Ganglion Cells (RGCs) following Ischemia-Reperfusion Injury.

FIGS. 23 and 24 show Optical Coherence Tomography (OCT) data obtained using Oxygen-Induced Retinopathy (OIR) model system. In this model, at 10 days after Oxygen-Induced Retinopathy (OIR), retinal thickness is defined by retinal layer cellularity: the thicker—the better.

FIGS. 25 and 26 show RGC counts obtained using Oxygen-Induced Retinopathy (OIR) model system for evaluation of protection of Retinal Ganglion Cells (RGCs) following Ischemia-Reperfusion Injury.

FIG. 27 shows the Experimental Design used in a Glaucoma Rat Model System for Evaluation of dsRNA targeting RTP801 Neuroprotective Activity. RGCs were counted in 12 quadrants per each retina whole mount with preceding fluorogold labeling via superior colliculus. Axons were counted in semi-thin optic nerve transversal sections (one section per nerve) in 5 areas (1 central, 2 ventral and 2 dorsal)

FIGS. 28 to 30 show intraocular pressure (IOP), RGC density and Axon counts, respectively, obtained in a Rat Glaucoma Model System for Evaluation of dsRNA targeting RTP801 Neuroprotective Activity.

FIGS. 31 to 33 show the neuroprotective effect of Casp2 dsRNA (“siCasp2+siEGFP” treatment group), RTP801 dsRNA (“siRTP801+siEGFP” treatment group), and their combination (“siCasp2+siRTP801” treatment group), as compared to the intact group and to a control (“siEGFP” treatment group) after administration by intravitreal (IVT) in Rat Axotomy Model at two (2) weeks post injury. Evaluation of the neuroprotective effects of each of the treatments was performed by counting of FG relabeled RGC in retinal whole mounts at 2 weeks after axotomy.

### DETAILED DESCRIPTION OF THE INVENTION

Provided herein are oligonucleotide compositions and methods of use thereof for treating various diseases, disorders and injury of a peripheral nervous system and a central nervous system, including a visual system, and an audio-vestibular system.

Further disclosed herein are pharmaceutical compositions that comprise a therapeutically effective amount of a therapeutic agent that down-regulates the expression of the RTP801 gene or the REDD2 gene, and an therapeutic agent that down regulates expression of the Casp2 gene, for use in the treatment of a subject suffering from medical condition associated with expression of those genes. In various preferred embodiments of the methods and compositions disclosed herein, the therapeutic agent is a nucleic agent molecule, preferably a double stranded nucleic acid compound, such as a dsRNA compound including small interfering RNA (siRNA) and small interfering nucleic acid (siNA).

In various embodiments the double stranded RNA compounds possess structures and modifications which increase activity, increase stability, minimize toxicity, reduce off target effects and/or reduce immune response when compared to an unmodified double stranded RNA compound; the modifications are beneficially applied to double stranded oligonucleotide sequences useful in preventing or attenuating target gene expression, in particular the RTP801 and/or Casp2 genes discussed herein.

The disclosure provides for combination therapy for all the conditions disclosed herein and in particular eye diseases and conditions involving neurodegeneration and/or requiring neuroprotection. In such combination therapy, both the RTP801 and Casp2 genes are inhibited in order to ameliorate the symptoms of the disease being treated. These genes may be inhibited with a combination of siRNAs or antibodies (including aptamer antibodies) or both. The present disclosure therefore also provides for a pharmaceutical composition comprising an RTP801 inhibitor and a Casp2 inhibitor, the RTP801 inhibitor preferably being a nucleic acid molecule, such as a double stranded RNA oligonucleotide, optionally an siRNA, more preferably an siRNA molecule detailed in Table A herein and in particular, an siRNA comprising the following antisense strand 5′ AGCUGCAUCAGGUUGGCAC 3′ (SEQ ID NO:7) known as REDD14, PF-655 and/or an siRNA targeting positions 450-500 or positions 1100-1130 or positions 1600-1650 of an RTP801 mRNA (SEQ ID NO:1); the Casp2 inhibitor preferably being a nucleic acid molecule, such as a double stranded RNA oligonucleotide, optionally an siRNA, more preferably an siRNA molecule detailed in Table B herein and in particular, an siRNA comprising the following antisense strand 5′ AGGAGUUCCACAUUCUGGC 3′ (SEQ ID NO:9) and the use of this composition in the preparation of a medicament for use in the therapy of conditions and disorders disclosed herein. In one embodiment the disorder involves ganglion death; in another embodiment the disorder involvers increased intra-ocular pressure. Preferred dsRNA compounds targeting RTP801 include an antisense sequence selected from SEQ ID NO:7, 15, 17, 19, 21, 23, 25, 27 or 29.

TABLE A
RTP801 dsRNA sequences useful for preparing compounds and
compositions according to the present disclosure

| | SEQ | | SEQ | | |
|---|---|---|---|---|---|
| | ID | Sense strand | ID | Antisense strand | Species |
| ID | NO: | (5′ > 3′) | NO: | (5′ > 3′) | targeted* |
| DDIT4_1 | 8 | GUGCCAACCUGAUGCAGCU | 7 | AGCUGCAUCAGGUUGGCAC | Hu, ms, rat |
| DDIT4_23 | 16 | CUGGGUCUUCCAUCUAGAA | 15 | UUCUAGAUGGAAGACCCAG | Hu, ms, rat |
| DDIT4_24 | 18 | GAAUACACUUGAUGUUCAA | 17 | UUGAACAUCAAGUGUAUUC | Hu, ms, rat |
| DEnT4_53 | 20 | AGACCUAUGCAAUAUUUUU | 19 | AAAAAUAUUGCAUAGGUCU | Hu, ms, rat |
| DEnT4_25 | 22 | UACACUUGAUGUUCAAGUA | 21 | UACUUGAACAUCAAGUGUA | Hu, ms, rat |
| DDIT4_2 | 24 | UACUGUAGCAUGAAACAAA | 23 | UUUGUUUCAUGCUACAGUA | Hu, Cyn, ms, rt |
| DDIT4_3 | 26 | CAGUACUGUAGCAUGAAAC | 25 | GUUUCAUGCUACAGUACUG | Hu, Cyn, ms, rt |
| DDIT4_32 | 28 | CCUCAGUACUGUAGCAUGA | 27 | UCAUGCUACAGUACUGAGG | Hu, Cyn, ms, rt |
| DDIT4_34 | 30 | CUCAGUACUGUAGCAUGAA | 29 | UUCAUGCUACAGUACUGAG | Hu, Cyn, ms, rt |

*hu: human; ms: mouse, cyn: cynamolgus, rt: rat

Additional information about these dsRNAs, as well as additional dsRNAs targeting RTP801 which are useful in the presently disclosed compositions and methods, are provided in PCT publication Nos. WO 2006/023544, WO 2007/084684 and WO2009/116037.

TABLE B
Casp2 sequences useful for preparing compounds and
compositions according to the present disclosure.

| | SEQ | | SEQ | |
|---|---|---|---|---|
| | ID | Sense strand | ID | Antisense strand |
| ID | NO: | (5′ > 3′) | NO: | (5′ > 3′) |
| CASP2_4 | 32 | GCCAGAAUGUGGAACUCCU | 31 | AGGAGUUCCACAUUCUGGC |
| CASP2_1 | 34 | GCACUCCUGAAUUUUAUCA | 33 | UGAUAAAAUUCAGGAGUGC |
| CASP2_2 | 36 | GCACAGGAAAUGCAAGAGA | 35 | UCUCUUGCAUUUCCUGUGC |
| CASP2_3 | 38 | GGGCUUGUGAUAUGCACGU | 37 | ACGUGCAUAUCACAAGCCC |

Additional information about these dsRNAs, as well as additional dsRNAs targeting Casp2, which are useful in the presently disclosed compositions and methods, are provided in PCT publication Nos. WO 2008/050329 and WO 2010/048352.

The conjoined administration of an RTP801 inhibitor, preferably a dsRNA, and a Casp2 inhibitor, preferably a dsRNA compound, can have a synergistic effect whereby said combined treatment is more effective than treatment by any of these individual compositions, irrespective of dosage in the single therapy option. This synergistic effect is also supported by preliminary results disclosed herein, as detailed in Example 1.

Without being bound by theory, RTP801 has a different mechanism of action than Casp2 and its inhibition is potentially synergistic with Casp2 inhibition.

In an additional embodiment, the disclosure provides for a single, double-stranded RNA compound which is processed, optionally by endogenous intra cellular complexes, to produce two or more double stranded RNA molecules which target RTP801 and Casp2, and a pharmaceutical composition comprising such oligonucleotide. Further provided is an RTP801 dsRNA, covalently or non-covalently bound to a Casp2 dsRNA; and a pharmaceutical composition comprising such bound dsRNAs. Also provided herein are tandem double-stranded compounds, which comprises two or more dsRNA sequences, at least one capable of inhibiting RTP801 or REDD2 and at least one capable of inhibiting Casp2; and a pharmaceutical composition comprising such compound. In some embodiments, the sequences of the RTP801 dsRNA and the Casp2 dsRNA detailed above are incorporated into these compounds.

Without being bound by theory, Casp2 is a pro-apoptotic gene that is specifically expressed and activated in retinal ganglion cells (RGC) following axonal injury. The assignee of the present application has previously demonstrated that down-regulation of Casp2 by intravitreal injection of the siRNA molecule known as QPI1007 in a rat models of optic nerve damage resulted in robust rescue of RGC from apoptotic death (Ahmed Z. et al. (2011). Ocular neuroprotection by siRNA targeting caspase-2. Cell Death Dis. June 16; 2:e173).

RTP801 is induced under conditions of hypoxia and oxidative stress and inhibits the mTOR pathway at the level of activation of the inhibitory TSC1/TSC2 complexes (Corradetti M N et al. (2005) The stress-inducted proteins RTP801 and RTP801L are negative regulators of the mammalian target of rapamycin pathway. J Biol. Chem. March 18; 280(11):9769-72.) RTP801 plays a negative role in neurogenesis during embryogenesis and negatively regulates neuronal survival in adulthood. Thus, knockdown of RTP801 in vitro and in vivo may accelerate cell cycle exit by neuroprogenitors and their differentiation into neurons (Malagelada C et al. (2011). RTP801/REDD1 regulates the timing of cortical neurogenesis and neuron migration. J. Neurosci. March 2; 31(9):3186-96.). RTP801 knockout mice display reduced apoptosis in the inner nuclear layer of the retina in the model of retinopathy of prematurity (Brafman A. et al. (2004) Inhibition of oxygen-induced retinopathy in RTP801-deficient mice. Invest Ophthalmol V is Sci. October; 45(10):3796-805.). RTP801 is strongly upregulated in brain substantia nigra neurons and its inhibition by shRNA protects neurons from death in the in vitro model of Parkinson's disease (PMID: 17005863). The assignee of the present application has previously demonstrated that inhibition of RTP801 by the siRNA known as REDD14 (a.k.a. PF-655) possesses a neuroprotective effect in vitro in RGC-5, a retinal ganglion cell line, treated with cobalt chloride or blue light (Garcia-Manso et al, ARV02011, 1424/A573). The mechanism underlying neuroprotective consequences of the inhibition of expression of RTP801 involves the downstream activation of the mTOR pathway with subsequent activation of pro-survival AKT kinase (PMID: 19118169; PMID: 21463685; PMID: 20230819) via activation of one of the mTOR containing signalling complexes, TORC2, that is rapamycin insensitive (PMID: 12408816; PMID: 17141160). In addition to neuroprotective activity, both the activation of mTOR pathway and PEDF have been demonstrated to positively regulate neurite outgrowth in retinal and other types of neurons and to promote neuronal regeneration (PMID: 17274541; PMID: 15647476; PMID: 18988856; PMID: 20062052). The assignee of the present application has demonstrated that siRNA-mediated inhibition of RTP801 paralogue, RTP801L (REDD2) [similar to RTP801, RTP801L also inhibits mTOR activity (PMID: 15632201)], following intravitreal siRNA injection in the mouse optic nerve axotomy model elicited restoration of retinal ganglion cell dendritic arbors and glutamatergic bipolar cell inputs onto RGC dendritic shafts (Morquette et al, ARVO 2011, 2689/A170).

As disclosed herein, inhibition of RTP801 or of REDD2 (RTP801L) contributes to neurite outgrowth and neuroregeneration. Particularly preferred REDD2 nucleic acid inhibitors are disclosed in U.S. Pat. No. 7,626,015 and US Patent Application Publication No. 20110105584 disclose dsRNA RTP801L (REDD2) molecules.

In one aspect, provided herein is a method of treating a subject at risk of or afflicted with a disease, disorder or injury comprising administering to the subject a therapeutically effective dose of an agent that down-regulates expression of a pro-apoptotic gene and a therapeutically effective dose of an agent that down regulates expression of RTP801 or of REDD2, thereby treating the subject.

In some embodiments of the method, the disease, disorder or injury is associated with degeneration or loss of function of the optic nerve and/or the retinal ganglion cells. In some embodiments the method provided herein, comprise rescuing a retinal ganglion cell from apoptosis in a subject, comprising dosing the subject with a therapeutically effective amount of at least two double stranded RNA compounds one which targets RTP801 or REDD2 and a second which targets Casp2, optionally in the retina of the subject, thereby rescuing retinal ganglion cell from apoptosis in the subject.

In some embodiments the method provides promoting survival of a retinal ganglion cell in a subject displaying signs or symptoms of an ocular neuropathy, comprising applying to the subject a therapeutically effective amount of a composition comprising two double stranded RNA molecules, one which targets RTP801 or REDD2 and a second which targets Casp2, thereby promoting survival of a retinal ganglion cell in the subject. In some embodiments the signs or symptoms are mediated by apoptosis. In one embodiment the method comprises administration of at least one dsRNA molecule that down regulates expression of RTP801 and further at least one dsRNA molecule that down regulates expression of Casp2. In another embodiment the method comprises administration of at least one dsRNA molecule that down regulates expression of REDD2 and of Casp2. In some embodiments retinal ganglion cell death is mediated by elevated intraocular pressure (IOP) in the eye of a subject or results from an ischemic event. In some embodiments, provided herein is a method of delaying, preventing or rescuing a retinal cell from death in a subject suffering from elevated IOP comprising applying to the eye of the subject a therapeutically effective amount of a double stranded RNA compound to the RTP801 gene or to REDD2 gene, and a therapeutically effective amount of a double stranded RNA compound to Casp2 gene, thereby delaying, preventing or rescuing the retinal cell from injury or death and wherein intraocular pressure (IOP) remains substantially elevated.

Further provided is a method for attenuating retinal ganglion cell loss and providing neuroprotection to a subject in need thereof, comprising administering to the subject a therapeutically effective amount of at least one double stranded RNA compound to the RTP801 gene or a REDD2 gene, and a therapeutically effective amount of at least one double stranded RNA compound to the Casp2 gene, thereby attenuating retinal ganglion cell loss and providing neuroprotection to the subject. In some embodiments the method comprises administering a double stranded RNA compound to the Casp2 gene and a double stranded RNA compound to the RTP801 gene.

Also provided is a method for preventing visual field loss associated with loss of retinal ganglion cells in a subject, comprising administering to the subject a composition comprising a therapeutically effective amount of at least one double stranded RNA compound to the RTP801 gene or the REDD2 gene, and a therapeutically effective amount of at least one double stranded RNA compound to the Casp2 gene, thereby preventing visual field loss in the subject.

Further disclosed is a method of preventing, treating or ameliorating a neurodegenerative disease, disorder or condition in a subject in need thereof, which comprises administering to the subject at least one double stranded RNA oligonucleotide directed to the RTP801 gene, or the REDD2 gene, and at least one double stranded RNA oligonucleotide directed to Casp2; thereby preventing, treating or ameliorating a neurodegenerative disease, disorder or condition in the subject.

In various embodiments disclosed herein, in which the pharmaceutical compositions are administered topically, the pharmaceutical compositions further comprise a permeability enhancer, also known as a penetration enhancer.

In some embodiments of the methods and compositions provided herein, the nucleic acid molecule is a double stranded RNA compound.

In some embodiments the dsRNA to the target genes disclosed herein comprises the following double stranded structure (A1):

| | | |
|---|---|---|
| (A1) | 5′ (N)x-Z 3′ | (antisense strand) |
| | 3′ Z′-(N′)y-z″ 5′ | (sense strand) |

wherein each N and N′ is a ribonucleotide which may be an unmodified ribonucleotide, modified ribonucleotide, or an unconventional moiety;
wherein each of (N)x and (N′)y is an oligonucleotide in which each consecutive N or N′ is joined to the next N or N′ by a covalent bond;
wherein each of Z and Z′ is independently present or absent, but if present independently comprises 1-5 consecutive nucleotides, 1-5 consecutive non-nucleotide moieties or a combination thereof covalently attached at the 3′ terminus of the strand in which it is present;
wherein z″ may be present or absent, but if present is a capping moiety covalently attached at the 5′ terminus of (N′)y;
wherein x=y=18-40;
wherein the sequence of (N′)y is complementary to the sequence of (N)x; and wherein (N)x comprises an antisense sequence and (N′)y comprises a consecutive sense sequence present in any one of SEQ ID NO:1-6.

In some embodiments of the double stranded oligonucleotide compound of Structure A1, x=y=19. In various embodiments both Z and Z′ are absent in the double stranded oligonucleotide compound; i.e. the double stranded compound is blunt ended on both ends. In some embodiments at least one of Z or Z′ is present in said double stranded oligonucleotide compound.

In some embodiments both Z and Z′ are present and each one is independently a non-nucleotide moiety. In some embodiments at least one of N or N′ in the double stranded oligonucleotide compound comprises a 2′ sugar modified ribonucleotide. In some embodiments the 2′ sugar modification comprises the presence of an amino, a fluoro, an alkoxy or an alkyl moiety. In some preferred embodiments 2′ sugar modification comprises the presence of an alkoxy moiety, preferably the alkoxy moiety comprises a 2′O-Methyl moiety. In some embodiments one or more pyrimidine nucleotides in the antisense strand comprises a 2′O-Methyl sugar modified ribonucleotide. In some embodiments all pyrimidine ribonucleotides in (N)ₓ comprise 2′O-Methyl sugar modified pyrimidine ribonucleotides. In various embodiments (N)ₓ comprises an L-DNA moiety at position 6 or 7 (5′>3′).

In some embodiments (N′)y comprises at least one unconventional moiety selected from a mirror nucleotide and a nucleotide joined to an adjacent nucleotide by a 2′-5′ internucleotide phosphate bond. In some embodiments the unconventional moiety in (N′)y is a mirror nucleotide. In some embodiments the mirror nucleotide in (N′)y is an L-deoxyribonucleotide (L-DNA). In some embodiments (N′)y consists of unmodified ribonucleotides at positions 1-17 and 19 and one L-DNA at the 3′ penultimate position (position 18). In some embodiments (N′)y consists of unmodified ribonucleotides at position 1-16 and 19 and two consecutive L-DNA at the 3′ penultimate position (positions 17 and 18). In some embodiments the unconventional moiety in (N′)y is a nucleotide joined to an adjacent nucleotide by a 2′-5′ internucleotide phosphate linkage. In some embodiments, in (N′)y the nucleotide joined to an adjacent nucleotide by a 2′-5′ internucleotide phosphate linkage further comprises a 3′-O-Methyl (3′O-Me) sugar modification.

In some embodiments the dsRNA to the target genes disclosed herein comprises the following double stranded structure (A2):

| | | |
|---|---|---|
| (A2) | 5′ N1-(N)x-Z 3′ | (antisense strand) |
| | 3′ Z′-N2-(N′)y-z″ 5′ | (sense strand) |

wherein each N1, N2, N and N′ is independently an unmodified ribonucleotide, a modified ribo nucleotide, or an unconventional moiety;
wherein each of (N)x and (N′)y is an oligonucleotide in which each consecutive N or N′ is joined to the adjacent N or N′ by a covalent bond;
wherein each of x and y is independently an integer between 17 and 39;
wherein N2 is covalently bound to (N′)y;
wherein N1 is covalently bound to (N)x and is mismatched to the target mRNA (SEQ ID NO:1-7) or is a complementary DNA moiety to the target mRNA;
wherein N1 is a moiety selected from the group consisting of natural or modified: uridine, deoxyribouridine, ribothymidine, deoxyribothymidine, adenosine or deoxyadenosine, an abasic ribose moiety and an abasic deoxyribose moiety;
wherein z″ may be present or absent, but if present is a capping moiety covalently attached at the 5′ terminus of N2-(N′)y;
wherein each of Z and Z′ is independently present or absent, but if present is independently 1-5 consecutive nucleotides, 1-5 consecutive non-nucleotide moieties or a combination thereof covalently attached at the 3′ terminus of the strand in which it is present; and
wherein the sequence of (N′)y is complementary to the sequence of (N)x; and wherein the sequence of N2-(N′)y comprises a consecutive sense sequence present in any one of SEQ ID NO:1-6.

In some embodiments of the double stranded oligonucleotide compound according to Structure (A)2, x=y=18.

In some embodiments N1 and N2 form a Watson-Crick base pair. In other embodiments N1 and N2 form a non-Watson-Crick base pair. In some embodiments N1 is a modified riboadenosine or a modified ribouridine.

In certain embodiments N1 is selected from the group consisting of riboadenosine, modified riboadenosine, deoxyriboadenosine, modified deoxyriboadenosine. In other embodiments N1 is selected from the group consisting of ribouridine, deoxyribouridine, modified ribouridine, and modified deoxyribouridine.

In certain embodiments N1 is selected from the group consisting of riboadenosine, modified riboadenosine, deoxyriboadenosine, modified deoxyriboadenosine and N2 is selected from the group consisting of ribouridine, deoxyribouridine, modified ribouridine, and modified deoxyribouridine. In certain embodiments N1 is selected from the group consisting of riboadenosine and modified riboadenosine and N2 is selected from the group consisting of ribouridine and modified ribouridine.

In certain embodiments N2 is selected from the group consisting of riboadenosine, modified riboadenosine, deoxyriboadenosine, modified deoxyriboadenosine and N1 is selected from the group consisting of ribouridine, deoxyribouridine, modified ribouridine, and modified deoxyribouridine. In certain embodiments N1 is selected from the group consisting of ribouridine and modified ribouridine and N2 is selected from the group consisting of riboadenine and modified riboadenine. In certain embodiments N1 is ribouridine and N2 is riboadenine. In certain embodiments N1 is riboadenine and N2 is ribouridine.

In specific embodiments of Structure A1 and/or Structure A2, each of N, N′, N1 and N2 is an unmodified ribonucleotide, z″ is absent, Z and Z′ are present and consist of dTdT overhang. In specific embodiments of Structure A1 and/or Structure A2, Z comprises at least one C3 alkyl overhang. In some embodiments the C3-C3 overhang is covalently attached to the 3′ terminus of (N)x or (N′)y via a covalent linkage, preferably a phosphodiester linkage. In some embodiments the linkage between a first C3 and a second C3 is a phosphodiester linkage. In some embodiments the 3′ non-nucleotide overhang is C3Pi-C3Pi. In some embodiments the 3′ non-nucleotide overhang is C3Pi-C3Ps. In some embodiments the 3′ non-nucleotide overhang is C3Pi-C3OH(OH is hydroxy). In some embodiments the 3′ non-nucleotide overhang is C3Pi-C3OH.

In various embodiments the alkyl moiety comprises an alkyl derivative including C3 alkyl, C4 alkyl, C5 alky or C6 alkyl moiety comprising a terminal hydroxyl, a terminal amino, or terminal phosphate group. In some embodiments the alkyl moiety is a C3 alkyl or C3 alkyl derivative moiety. In some embodiments the C3 alkyl moiety comprises propanol, propylphosphate, propylphosphorothioate or a combination thereof. The C3 alkyl moiety is covalently linked to the 3′ terminus of (N′)y and/or the 3′ terminus of (N)x via a phosphodiester bond. In some embodiments the alkyl moiety comprises propanol, propyl phosphate or propyl phosphorothioate. In some embodiments each of Z and Z′ is independently selected from propanol, propyl phosphate propyl phosphorothioate, combinations thereof or multiples thereof in particular 2 or 3 covalently linked propanol, propyl phosphate, propyl phosphorothioate or combinations thereof. In some embodiments each of Z and Z′ is independently selected from propyl phosphate, propyl phosphorothioate, propyl phospho-propanol; propyl phospho-propyl phosphorothioate; propylphospho-propyl phosphate; (propyl phosphate)3, (propyl phosphate)2-propanol, (propyl phosphate)2-propyl phosphorothioate. Any propane or propanol conjugated moiety can be included in Z or Z′.

The structures of exemplary 3′ terminal non-nucleotide moieties are as follows:

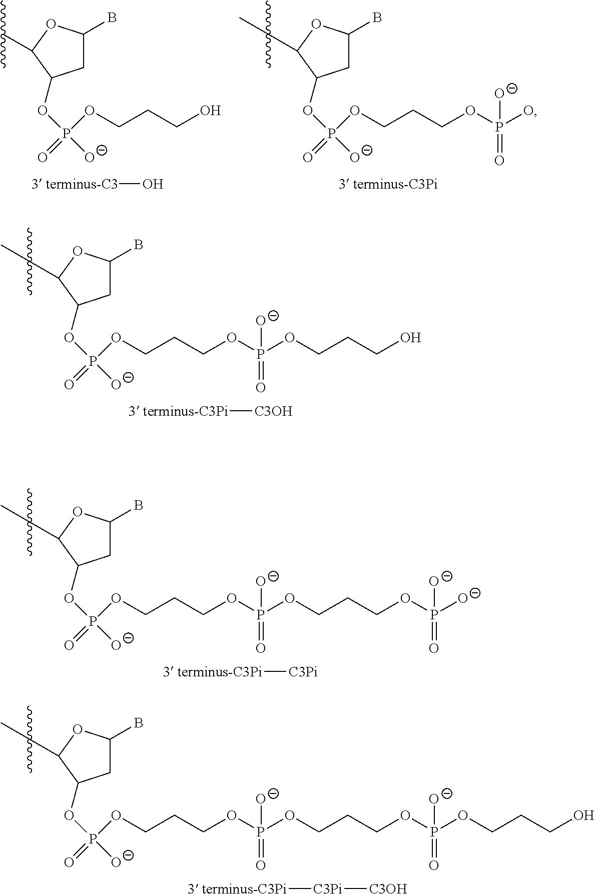

Throughout the specification, nucleotide positions are numbered from 1 to 19 and are counted from the 5′ end of the antisense strand or the sense strand. For example, position 1 on (N)x refers to the 5′ terminal nucleotide on the antisense oligonucleotide strand and position 1 on (N′)y refers to the 5′ terminal nucleotide on the sense oligonucleotide strand. PCT Patent Publications WO 2011/066475 and WO 2011/085056, assigned to the assignee of the present application and incorporated herein by reference in their entirety disclose compositions and methods useful for generating dsRNA molecules.

In another aspect, provided herein is a method of promoting neurite outgrowth, axonal regeneration or neural regeneration, comprising contacting a neuron with an effective amount of an RTP801 inhibitor or a salt thereof, or with an effective amount of a REDD2 inhibitor or a salt thereof, thereby promoting neurite outgrowth, axonal regeneration or neural regeneration. This method may also be performed in vitro/ex vivo in cell culture which can optionally subsequently be returned to the body of the subject.

In some embodiments the subject of the methods disclosed herein is a mammal, preferably a human.

In a further aspect, provided herein is a method of treating a disease, a disorder or an injury of the CNS in a subject in need thereof, which comprises topically administering to the ear canal, eye, or skin of the subject a pharmaceutical composition, comprising at least two oligonucleotides directed to RTP801, or REDD2, and Casp2 or any combination of two of these genes or all three of these genes, in an amount and over a period of time effective to treat the subject.

In some embodiments provided herein is a method of treating a disease disorder or injury of the CNS. In some embodiments the disorder or disease is a neurodegenerative disease or disorder. In various embodiments the neurodegenerative disorder is selected from neurodegenerative conditions causing problems with movements, such as impairment of motor, sensory or autonomic function; and conditions affecting memory and related to cognitive impairment or dementia.

According to another aspect, provided herein is a pharmaceutical composition comprising a therapeutically effective amount of an agent that down regulates expression of RTP801 or REDD2 and a therapeutically effective amount of an agent that down regulates expression of Casp2; and a carrier, or mixtures thereof. In some embodiments the composition further includes a permeability enhancer. In some embodiments the carrier is a pharmaceutically acceptable excipient.

In preferred embodiments the at least one oligonucleotide compound is a double stranded RNA compound. In some preferred embodiments the at least one oligonucleotide compound is a chemically modified siRNA.

The pharmaceutical product disclosed herein may, for example, be a pharmaceutical composition comprising the first and second therapeutic agent in admixture in a pharmaceutically acceptable carrier. Alternatively, the pharmaceutical product may, for example, be a kit comprising a preparation of the first therapeutic agent and a preparation of the second therapeutic agent and, optionally, instructions for the simultaneous, sequential or separate administration of the preparations to a patient in need thereof.

Examples of oligonucleotide sequence pairs are provided in PCT Patent Publication Nos. WO 2006/023544, WO 2007/084684, WO 2008/050329, WO 2007/141796, WO 2009/044392, WO 2008/106102, WO 2008/152636, WO 2009/001359, WO 2009/090639 assigned to the assignee of the present application and incorporated herein by reference in their entirety.

### DEFINITIONS

For convenience certain terms employed in the specification, examples and claims are described herein.

It is to be noted that, as used herein, the singular forms “a”, “an” and “the” include plural forms unless the content clearly dictates otherwise.

Where aspects or embodiments are described in terms of Markush groups or other grouping of alternatives, those skilled in the art will recognize that the disclosure is also thereby described in terms of any individual member or subgroup of members of the group.

An “inhibitor” is a compound, which is capable of reducing (partially or fully) the expression of a gene or the activity of the product of such gene to an extent sufficient to achieve a desired biological or physiological effect. The term “inhibitor” as used herein refers to a siRNA inhibitor.

A “double stranded nucleic acid molecule” “double stranded RNA inhibitor” is a compound, which is capable of reducing the expression of a gene or the activity of the product of such gene to an extent sufficient to achieve a desired biological or physiological effect. The term as used herein refers to one or more of a siRNA, shRNA, and synthetic shRNA. Inhibition may also be referred to as down-regulation or, for RNAi, silencing.

The term “inhibit” as used herein refers to reducing the expression of a gene or the activity of the product of such gene to an extent sufficient to achieve a desired biological or physiological effect. Inhibition is either complete or partial. For example “inhibition” of APP gene means inhibition of the gene expression (transcription or translation) or polypeptide activity of one or more of the variants or an SNP (single nucleotide polymorphism) thereof.

Nucleic acid molecule(s) and/or methods as disclosed herein may be used to down regulate the expression of gene(s) that encode RNA referred to, by for example, Genbank Accession NM_{—}019058.2 (RTP801 also known as REDD1 and DNA-damage-inducible transcript 4 (DDIT4), having gene identifier gi|56676369|); and Genbank accession NM_{—}032982.2 and NM_{—}032983.2 (Casp2, caspase 2, apoptosis-related cysteine peptidase) having gene identifiers gi|39995058| and gi|39995060|, respectively. SEQ ID NO:1 sets forth the mRNA polynucleotide sequence for RTP801 (REDD1; DDIT4); SEQ ID NO:2-4 sets forth the mRNA polynucleotide sequences for CASP2 splice variants; SEQ ID NO:6 sets forth the mRNA polynucleotide sequence for RTP801L (REDD2, DDIT4L).

A “pro-apoptotic gene” is defined as gene that plays a role in apoptotic cell death, either directly or indirectly. Pro-apoptotic genes include Caspases (Casp1, Casp2, Casp3, Casp4, Casp5, Casp6, Casp7, Casp8, Casp9, Casp10, Casp12, Casp14), Apaf1, NLRC4 (IPAF), NLRP1, NLRP2, CRADD, RIPK1, RIPK2, RIPK3, BCL10, PYCARD, CARDS, FADD, DEDD, NLRP3, FAS, FASL, PYDC1, TRADD, ZUD, TNFRSF1A, PIDD, DAPK1, DAPK2, STK17A, AIF, AMID, HTRA2, GZMB (granzyme B), FAF1, TNFRSF10A, TNFRSF10B, Bax, Puma, ASPP1, ASPP2, NOX2, NOX 4, p75NTR (NGFR) and RhoA.

“Eye disease” refers to conditions, diseases or syndromes of the eye including but not limited to any conditions involving choroidal neovascularization (CNV), wet and dry AMD, ocular histoplasmosis syndrome, angiod streaks, ruptures in Bruch's membrane, myopic degeneration, ocular tumors, retinal degenerative diseases and retinal vein occlusion (RVO). Some conditions disclosed herein, such as DR, which may be treated according to the methods disclosed herein have been regarded as either a microvascular disorder or an eye disease, or both, under the definitions presented herein.

“Central nervous system” or “CNS” means the brain, optic nerve, retina and/or spinal cord.

As used herein, “central nervous system disorder” or “CNS disorder” or “disease of the central nervous system” or “disease of the CNS” means any condition or disease that causes or results in a functional and/or physical deficit in the brain, retina, optic nerve and/or spinal cord or in the cells and tissues which comprise the brain, retina, optic nerve and/or spinal cord.

The term “injury of the central nervous system” or “injury of the CNS” refers to any and all injury or trauma of the brain, retina, optic nerve and/or spinal cord, including traumatic and non-traumatic injury, that causes or results in an impairment of motor and/or sensory and/or cognitive and/or mental and/or emotional and/or autonomic function.

As used herein, the term “neuroprotection” means the arrest and/or slow down and/or attenuate and/or reverse progression of neurodegeneration. As used herein, the term “neurodegeneration” means the progressive loss of neurons. This includes but is not limited to immediate loss of neurons followed by subsequent loss of connecting or adjacent neurons.

“Neuron,” “neuronal cell” and “neural cell” (including neural progenitor cells and neural stem cells) are used interchangeably to refer to nerve cells, i.e., cells that are responsible for conducting nerve impulses from one part of the body to another. Most neurons consist of three distinct portions: a cell body which contains the nucleus, and two different types of cytoplasmic processes: dendrites and axons. Dendrites, which are the receiving portion of the neuron, are usually highly branched, thick extensions of the cell body. The axon is typically a single long, thin process that is specialized to conducts nerve impulses away from the cell body to another neuron or muscular or glandular tissue. Axons may have side branches called “axon collaterals.” Axon collaterals and axons may terminate by branching into many fine filaments called telodendria. The distal ends of telodendria are called synaptic end bulbs or axonal terminals, which contain synaptic vesicles that store neurotransmitters. Axons may be surrounded by a multilayered, white, phospholipid, segmented covering called the myelin sheath, which is formed by Schwann cells in the peripheral nervous system and oligodendrocytes in the central nervous system. Axons containing such a covering are “myelinated.” “Axonogenesis” refers to the growth and differentiation of axonal processes by developing neurons. Neurons include sensory (afferent) neurons, which transmit impulses from receptors in the periphery to the brain and spinal cord and from lower to higher centers of the central nervous system. A neuron can also be motor (efferent) neurons which convey impulses from the brain and spinal cord to effectors in the periphery and from higher to lower centers of the central nervous system. Other neurons are association (connecting or interneuron) neurons which carry impulses from sensory neurons to motor neurons and are located within the central nervous system. The processes of afferent and efferent neurons arranged into bundles are called “nerves” when located outside the CNS or fiber tracts if inside the CNS.

“Axon growth” or “axonal growth” includes axon extension, axon regeneration and axon elongation.

The term “topical administration” or “topical application” is used to mean a local administration of a composition, preferably to the eye of the subject but also optionally to the ear, skin or any other organ where topical administration is relevant.

The term “otic” and “auricular” are used herein interchangeably and generally refer to tissue in and/or around an ear, including the outer ear, the middle ear and the inner ear.

The term “ear canal” or “external auditory meatus” is used to mean a tube running from the outer ear to the middle ear.

The “tympanic membrane” (also tympanum or myrinx) refers to a thin membrane that separates the external ear from the middle ear.

Terms such as “pharmaceutical composition” or “otic pharmaceutical composition” or “ocular pharmaceutical composition” “pharmaceutical formulation” or “pharmaceutical preparation” are used herein interchangeably to generally refer to formulations that are adapted to administration and delivery of one or more oligonucleotide active compounds to the eye, CNS, a CNS cell, a group of CNS cells, or a CNS tissue, in an animal or a human.

“Treatment,” “treat,” or “treating,” as used herein covers any treatment of a disease or condition of a mammal, particularly a human, and includes: (a) preventing the disease or condition from occurring in a subject which may be at risk of developing or predisposed to the disease or condition but has not yet been diagnosed as having it; (b) inhibiting the disease or condition, i.e., arresting or slowing down or postponing its development or progression; (c) relieving and/or ameliorating the disease or condition, i.e., causing regression of the disease or condition and/or the symptoms thereof; or (d) curing the disease or condition, i.e., stopping its development or progression. The population of subjects treated by the methods disclosed herein includes subjects suffering from the undesirable condition or disease, as well as subjects at risk for development of the condition or disease.

As used herein, the term “pharmaceutically acceptable” means that the components, in addition to the therapeutic agent, comprising the formulation, are suitable for administration to the patient being treated in accordance with the present disclosure.

A “penetration enhancer” or “permeability enhancer” refers to a compound or a combination of compounds that enhance the penetration of a therapeutic oligonucleotide through the retina, skin and/or the tympanic membrane in the ear of an animal or a human.

As used herein, the term “tissue” refers to an aggregation of similarly specialized cells united in the performance of a particular function.

“CNS cells” includes one or more of neuronal cells and/or glial cells (e.g. oligodendrocytes, astrocytes, ependymal cells, microglial cells, radial glia cells, or Schwann cells) and include the optic nerve and cells of the retina.

The term “antibody” refers to IgG, IgM, IgD, IgA, and IgE antibody, inter alia. The definition includes polyclonal antibodies or monoclonal antibodies. This term refers to whole antibodies or fragments of antibodies comprising an antigen-binding domain, e.g. antibodies without the Fc portion, single chain antibodies, miniantibodies, fragments consisting of essentially only the variable, antigen-binding domain of the antibody, etc. The term “antibody” may also refer to antibodies against polynucleotide sequences obtained by cDNA vaccination. The term also encompasses antibody fragments which retain the ability to selectively bind with their antigen or receptor and are exemplified as follows, inter alia:
(1) Fab, the fragment which contains a monovalent antigen-binding fragment of an antibody molecule which can be produced by digestion of whole antibody with the enzyme papain to yield a light chain and a portion of the heavy chain;
(2) (Fab′)₂, the fragment of the antibody that can be obtained by treating whole antibody with the enzyme pepsin without subsequent reduction; F(ab′₂) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.

By the term “epitope” as used in this disclosure is meant an antigenic determinant on an antigen to which the antibody binds. Epitopic determinants usually consist of chemically active surface groupings of molecules such as amino acids or sugar side chains and usually have specific three-dimensional structural characteristics, as well as specific charge characteristics.

### Preparation of Anti-RTP801 Antibodies

Antibodies which bind to RTP801 or a fragment derived therefrom may be prepared using an intact polypeptide or fragments containing smaller polypeptides as the immunizing antigen. For example, it may be desirable to produce antibodies that specifically bind to the N- or C-terminal or any other suitable domains of the RTP801. The polypeptide used to immunize an animal can be derived from translated cDNA or chemical synthesis and can be conjugated to a carrier protein, if desired. Such commonly used carriers which are chemically coupled to the polypeptide include keyhole limpet hemocyanin (KLH), thyroglobulin, bovine serum albumin (BSA) and tetanus toxoid. The coupled polypeptide is then used to immunize the animal.

If desired, polyclonal or monoclonal antibodies can be further purified, for example by binding to and elution from a matrix to which the polypeptide or a peptide to which the antibodies were raised is bound. Those skilled in the art know various techniques common in immunology for purification and/or concentration of polyclonal as well as monoclonal antibodies (Coligan et al, Unit 9, Current Protocols in Immunology, Wiley Interscience, 1994).

REDD2 and Casp2 antibodies can be prepared in a similar manner.

Methods for making antibodies of all types, including fragments, are known in the art (See for example, Harlow and Lane, Antibodies: A Laboratory Manual, Cold Spring Harbor Laboratory, New York (1988)). Methods of immunization, including all necessary steps of preparing the immunogen in a suitable adjuvant, determining antibody binding, isolation of antibodies, methods for obtaining monoclonal antibodies, and humanization of monoclonal antibodies are all known to the skilled artisan

The antibodies may be humanized antibodies or human antibodies. Antibodies can be humanized using a variety of techniques known in the art including CDR— grafting (EP239,400: PCT publication WO91/09967; U.S. Pat. Nos. 5,225,539; 5,530,101; and 5,585,089, veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,332).

The monoclonal antibodies as defined include antibodies derived from one species (such as murine, rabbit, goat, rat, human, etc.) as well as antibodies derived from two (or more) species, such as chimeric and humanized antibodies.

Completely human antibodies are particularly desirable for therapeutic treatment of human patients. Human antibodies can be made by a variety of methods known in the art including phage display methods using antibody libraries derived from human immunoglobulin sequences. See also U.S. Pat. Nos. 4,444,887 and 4,716,111; and PCT publications WO 98/46645, WO 98/50433, WO 98/24893, WO 98/16654, WO 96/34096, WO 96/33735, and WO 91/10741, each of which is incorporated herein by reference in its entirety.

Additional information regarding all types of antibodies, including humanized antibodies, human antibodies and antibody fragments can be found in WO 01/05998, which is incorporated herein by reference in its entirety.

Neutralizing antibodies can be prepared by the methods discussed above, possibly with an additional step of screening for neutralizing activity by, for example, a survival assay.

As used herein, the terms “polynucleotide” refers to nucleotide sequences comprising deoxyribonucleic acid (DNA), and ribonucleic acid (RNA). The terms are to be understood to include, as equivalents, analogs of either RNA or DNA made from nucleotide analogs. Throughout this application, mRNA sequences are set forth as representing the corresponding genes.

“Nucleic acid molecule”, “oligonucleotide” and “oligomer” are used interchangeably and refer to a deoxyribonucleotide or ribonucleotide sequence from about 2 to about 50 nucleotides. Each DNA or RNA nucleotide may be independently natural or synthetic, and or modified or unmodified. Modifications include changes to the sugar moiety, the base moiety and or the linkages between nucleotides in the oligonucleotide. The compounds of the present disclosure encompass molecules comprising deoxyribonucleotides, ribonucleotides, modified deoxyribonucleotides, modified ribonucleotides, unconventional moieties and combinations thereof. Oligonucleotide is meant to encompass single stranded molecules including antisense and shRNA, and double stranded molecules including double stranded RNA (dsRNA), siNA, siRNA and miRNA.

Substantially complementary refers to complementarity of greater than about 84%, to another sequence. For example in a duplex region consisting of 19 base pairs one mismatch results in 94.7% complementarity, two mismatches results in about 89.5% complementarity and 3 mismatches results in about 84.2% complementarity, rendering the duplex region substantially complementary. Accordingly substantially identical refers to identity of greater than about 84%, to another sequence.

The present disclosure provides methods and compositions for inhibiting expression of the RTP801 gene, or the REDD2 gene, and the Casp2 gene in vivo. In general, the methods include topical ocular administration of oligoribonucleotides, in particular double stranded RNA compounds (e.g. small interfering RNAs or siRNAs) which target RTP801 (or REDD2) and Casp2, or a nucleic acid material that can produce siRNA in a cell, in an amount sufficient to down-regulate expression of RTP801 or REDD2, and of Casp2 by an RNA interference mechanism. In accordance with the present disclosure, the double stranded RNA molecules or inhibitors of the RTP801 or REDD2, and Casp2 gene are used as drugs to treat various eye and CNS pathologies.

“Nucleotide” is meant to encompass deoxyribonucleotides and ribonucleotides, which may be natural or synthetic, and or modified or unmodified. Modifications include changes to the sugar moiety, the base moiety and or the linkages between ribonucleotides in the oligoribonucleotide. As used herein, the term “ribonucleotide” encompasses natural and synthetic, unmodified and modified ribonucleotides. Modifications include changes to the sugar moiety, to the base moiety and/or to the linkages between ribonucleotides in the oligonucleotide.

The nucleotides can be selected from naturally occurring or synthetic modified bases. Naturally occurring bases include adenine, guanine, cytosine, thymine and uracil. In some embodiments of the methods and compositions provided herein, the nucleic acid molecule comprises one or more modified nucleobases; independently selected from, without being limited to, xanthine, hypoxanthine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 8-halo, amino, thiol, thioalkyl, hydroxyl and other 8-substituted adenines and guanines, 5-trifluoromethyl and other 5-substituted uracils and cytosines, 7-methylguanine, and acyclonucleotides.

In various embodiments of the methods and compositions provided herein, the nucleic acid molecule comprises one or more modifications to the phosphodiester backbone. In some embodiments of the methods and compositions provided herein, the nucleic acid molecule comprises one or more modifications to the phosphodiester backbone; selected from, without being limited to, a phosphorothioate, 3′-(or -5′)deoxy-3′-(or -5′)thio-phosphorothioate, phosphorodithioate, phosphoroselenates, 3′-(or -5′)deoxy phosphinates, borano phosphates, 3′-(or -5′)deoxy-3′-(or 5′-)amino phosphoramidates, hydrogen phosphonates, borano phosphate esters, phosphoramidates, alkyl or aryl phosphonates and phosphotriester or phosphorus linkages.

In some embodiments of the methods and compositions provided herein, the nucleic acid molecule comprises one or more modifications in the sense strand but not the antisense strand. In some embodiments of the methods and compositions provided herein, the nucleic acid molecule comprises one or more modifications in the antisense strand but not the sense strand. In some embodiments of the methods and compositions provided herein, the nucleic acid molecule comprises one or more modifications in both the sense strand and the antisense strand. In some embodiments of the methods and compositions provided herein, the nucleic acid molecule comprises a pattern of alternating modifications in the sense strand, in the antisense strand, or in both, the sense strand and the antisense strand. In some embodiments of the methods and compositions provided herein, the nucleic acid molecule comprises a pattern of alternating 2′-O-methyl sugar modified nucleotides and unmodified nucleotides, in the sense strand, in the antisense strand, or in both, the sense strand and the antisense strand. In some embodiments of the methods and compositions provided herein, the nucleic acid molecule comprises a pattern of alternating 2′-O-methyl sugar modified nucleotides and unmodified nucleotides, in the sense strand, in the antisense strand, or in both, the sense strand and the antisense strand; and the pattern is configured such that modified nucleotides of the sense strand are opposite unmodified nucleotides in the antisense strand and vice-versa.

In some embodiments of the methods and compositions provided herein the nucleic acid molecule is a double stranded molecule and has a blunt end on both ends. In some embodiments of the methods and compositions provided herein the nucleic acid molecule is a double stranded molecule and has an overhang on both ends of the molecule. In some embodiments of the methods and compositions provided herein, the nucleic acid molecule is a double stranded molecule and has an overhang on both ends of the molecule; wherein said overhangs are 1-8 nucleotides in length. In some embodiments of the methods and compositions provided herein, the nucleic acid molecule is a double stranded molecule and has an overhang on both ends of the molecule; wherein said overhangs are 2 nucleotides in length. In some embodiments of the methods and compositions provided herein, the nucleic acid molecule is a double stranded molecule and has a 3′-overhang on both ends of the molecule. In some embodiments of the methods and compositions provided herein, the nucleic acid molecule is a double stranded molecule and has a 3′-overhang on both ends of the molecule; wherein said overhangs are 2 nucleotides in length. In some embodiments of the methods and compositions provided herein, the nucleic acid molecule is a double stranded molecule and has a 5′-overhang on both ends of the molecule. In some embodiments of the methods and compositions provided herein, the nucleic acid molecule is a double stranded molecule and has a 5′-overhang on both ends of the molecule; wherein said overhangs are 2 nucleotides in length. In some embodiments of the methods and compositions provided herein, the nucleic acid molecule is a double stranded molecule and has a blunt end on one end of the molecule and an overhang on the other end of the molecule; wherein said overhang is a 5′-overhang. In various embodiments of the methods and compositions provided herein, the overhang nucleotides in the nucleic acid molecule are modified nucleotides.

In some embodiments of the present disclosure the inhibitory oligonucleotide compound comprises unmodified and modified nucleotides and/or unconventional moieties. The compound comprises at least one modified nucleotide selected from the group consisting of a sugar modification, a base modification and an internucleotide linkage modification and may contain DNA, and modified nucleotides such as LNA (locked nucleic acid), ENA (ethylene-bridged nucleic acid), PNA (peptide nucleic acid), arabinoside, phosphonocarboxylate or phosphinocarboxylate nucleotide (PACE nucleotide), mirror nucleotide, or nucleotides with a 6 carbon sugar.

All analogs of, or modifications to, a nucleotide/oligonucleotide are employed with the present disclosure, provided that said analog or modification does not substantially adversely affect the function of the nucleotide/oligonucleotide. Acceptable modifications include modifications of the sugar moiety, modifications of the base moiety, modifications in the internucleotide linkages and combinations thereof.

A sugar modification includes a modification on the 2′ moiety of the sugar residue and encompasses amino, fluoro, alkoxy e.g. methoxy, alkyl, amino, fluoro, chloro, bromo, CN, CF, imidazole, carboxylate, thioate, C₁ to C₁₀ lower alkyl, substituted lower alkyl, alkaryl or aralkyl, OCF₃, OCN; O—, S—, or N— alkyl; O—, S, or N-alkenyl; SOCH₃; SO₂CH₃; ONO₂; NO₂, N₃; heterocycloalkyl; heterocycloalkaryl; aminoalkylamino; polyalkylamino or substituted silyl, as, among others, described in European patents EP 0 586 520 B1 or EP 0 618 925 B1.

In one embodiment the double stranded RNA compound comprises at least one ribonucleotide comprising a 2′ modification on the sugar moiety (“2′ sugar modification”). In certain embodiments the compound comprises 2′O-alkyl or 2′-fluoro or 2′O-allyl or any other 2′ modification, optionally on alternate positions. Other stabilizing modifications are also possible (e.g. terminal modifications). In some embodiments a preferred 2′O-alkyl is 2′O-methyl (methoxy) sugar modification.

In some embodiments the backbone of the oligonucleotides is modified and comprises phosphate-D-ribose entities but may also contain thiophosphate-D-ribose entities, triester, thioate, 2′-5′ bridged backbone (also may be referred to as 5′-2′), PACE and the like.

As used herein, the terms “non-pairing nucleotide analog” means a nucleotide analog which comprises a non-base pairing moiety including but not limited to: 6 des amino adenosine (Nebularine), 4-Me-indole, 3-nitropyrrole, 5-nitroindole, Ds, Pa, N3-Me ribo U, N3-Me riboT, N3-Me dC, N3-Me-dT, N1-Me-dG, N1-Me-dA, N3-ethyl-dC, N3-Me dC. In some embodiments the non-base pairing nucleotide analog is a ribonucleotide. In other embodiments it is a deoxyribonucleotide. In addition, analogues of polynucleotides may be prepared wherein the structure of one or more nucleotide is fundamentally altered and better suited as therapeutic or experimental reagents. An example of a nucleotide analogue is a peptide nucleic acid (PNA) wherein the deoxyribose (or ribose) phosphate backbone in DNA (or RNA) is replaced with a polyamide backbone which is similar to that found in peptides. PNA analogues have been shown to be resistant to enzymatic degradation and to have enhanced stability in vivo and in vitro. Other modifications useful in synthesizing oligonucleotides include polymer backbones, cyclic backbones, acyclic backbones, thiophosphate-D-ribose backbones, triester backbones, thioate backbones, 2′-5′ linked backbone (also known as 2′5′ nucleotides, or 2′5′ ribonucleotides [with 3′OH]), artificial nucleic acids, morpholino nucleic acids, glycol nucleic acid (GNA), threose nucleic acid (TNA), arabinoside, and mirror nucleoside (for example, beta-L-deoxyribonucleoside instead of beta-D-deoxyribonucleoside). Examples of siRNA compounds comprising LNA nucleotides are disclosed in Elmen et al., (NAR 2005, 33(1):439-447).

In some embodiments the double stranded RNA compounds are synthesized using one or more inverted nucleotides, for example inverted thymidine or inverted adenine (see, for example, Takei, et al., 2002, JBC 277(26):23800-06).

Other modifications include terminal modifications on the 5′ and/or 3′ part of the oligonucleotides and are also known as capping moieties. Such terminal modifications are selected from a nucleotide, a modified nucleotide, a lipid, a peptide, a sugar and inverted abasic moiety.

What is sometimes referred to in the present disclosure as an “abasic nucleotide” or “abasic nucleotide analog” is more properly referred to as a pseudo-nucleotide or an unconventional moiety. A nucleotide is a monomeric unit of nucleic acid, consisting of a ribose or deoxyribose sugar, a phosphate, and a base (adenine, guanine, thymine, or cytosine in DNA; adenine, guanine, uracil, or cytosine in RNA). A modified nucleotide comprises a modification in one or more of the sugar, phosphate and or base. The abasic pseudo-nucleotide lacks a base, and thus is not strictly a nucleotide.

The term “capping moiety” as used herein (“z″”) includes abasic ribose moiety, abasic deoxyribose moiety, modified abasic ribose and abasic deoxyribose moieties including 2′ O alkyl modifications; inverted abasic ribose and abasic deoxyribose moieties and modifications thereof; C6-imino-Pi; a mirror nucleotide including L-DNA and L-RNA; 5′O-Me nucleotide; and nucleotide analogs including 4′,5′-methylene nucleotide; 1-(β-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non bridging methylphosphonate and 5′-mercapto moieties.

Certain preferred capping moieties are abasic ribose or abasic deoxyribose moieties; inverted abasic ribose or abasic deoxyribose moieties; C6-amino-Pi; a mirror nucleotide including L-DNA and L-RNA. Another preferred capping moiety is a C3 non-nucleotide moiety derived from propanediol

The term “unconventional moiety” as used herein refers to abasic ribose moiety, an abasic deoxyribose moiety, a deoxyribonucleotide, a modified deoxyribonucleotide, a mirror nucleotide, a non-base pairing nucleotide analog and a nucleotide linked to an adjacent nucleotide by a 2′-5′ internucleotide phosphate bond; bridged nucleic acids including LNA and ethylene bridged nucleic acids.

In some embodiments of the present disclosure, a preferred unconventional moiety is an abasic ribose moiety, an abasic deoxyribose moiety, a deoxyribonucleotide, a mirror nucleotide, and a nucleotide linked to an adjacent nucleotide by a 2′-5′ internucleotide phosphate bond.

Abasic deoxyribose moiety includes for example abasic deoxyribose-3′-phosphate; 1,2-dideoxy-D-ribofuranose-3-phosphate; 1,4-anhydro-2-deoxy-D-ribitol-3-phosphate. Inverted abasic deoxyribose moiety includes inverted deoxyriboabasic; 3′, 5′ inverted deoxyabasic 5′-phosphate.

A “mirror nucleotide” is a nucleotide with reversed chirality to the naturally occurring or commonly employed nucleotide, i.e., a mirror image (L-nucleotide) of the naturally occurring (D-nucleotide), also referred to as L-RNA in the case of a mirror ribonucleotide, and “spiegelmer”. The nucleotide can be a ribonucleotide or a deoxyribonucleotide and my further comprise at least one sugar, base and or backbone modification. See U.S. Pat. No. 6,586,238. Also, U.S. Pat. No. 6,602,858 discloses nucleic acid catalysts comprising at least one L-nucleotide substitution. Mirror nucleotide includes for example L-DNA (L-deoxyriboadenosine-3′-phosphate (mirror dA); L-deoxyribocytidine-3′-phosphate (mirror dC); L-deoxyriboguanosine-3′-phosphate (mirror dG); L-deoxyribothymidine-3′-phosphate (mirror image dT)) and L-RNA (L-riboadenosine-3′-phosphate (mirror rA); L-ribocytidine-3′-phosphate (mirror rC); L-riboguanosine-3′-phosphate (mirror rG); L-ribouracil-3′-phosphate (mirror rU).

Modified deoxyribonucleotide includes, for example 5′OMe DNA (5-methyl-deoxyriboguanosine-3′-phosphate) which may be useful as a nucleotide in the 5′ terminal position (position number 1); PACE (deoxyriboadenine 3′ phosphonoacetate, deoxyribocytidine 3′ phosphonoacetate, deoxyriboguanosine 3′ phosphonoacetate, deoxyribothymidine 3′ phosphonoacetate).

Bridged nucleic acids include LNA (2′-O, 4′-C-methylene bridged Nucleic Acid adenosine 3′ monophosphate, 2′-O,4′-C-methylene bridged Nucleic Acid 5-methyl-cytidine 3′ monophosphate, 2′-O,4′-C-methylene bridged Nucleic Acid guanosine 3′ monophosphate, 5-methyl-uridine (or thymidine) 3′ monophosphate); and ENA (2′-O,4′-C-ethylene bridged Nucleic Acid adenosine 3′ monophosphate, 2′-O,4′-C-ethylene bridged Nucleic Acid 5-methyl-cytidine 3′ monophosphate, 2′-O,4′-C-ethylene bridged Nucleic Acid guanosine 3′ monophosphate, 5-methyl-uridine (or thymidine) 3′ monophosphate). According to one embodiment the methods disclosed herein provide for the use of inhibitory oligonucleotide compounds comprising unmodified and modified nucleotides. In various embodiments the compounds comprise at least one modified nucleotide selected from the group consisting of a sugar modification, a base modification and an internucleotide linkage modification and may contain DNA, and modified nucleotides such as LNA (locked nucleic acid) including ENA (ethylene-bridged nucleic acid; PNA (peptide nucleic acid); arabinoside; PACE (phosphonoacetate and derivatives thereof), mirror nucleotide, or nucleotides with a six-carbon sugar.

Any of the modifications disclosed herein can be employed in the preparation of the oligonucleotides which are incorporated into the compositions disclosed herein. Preferred modification schemes are disclosed, for examples, in PCT Publication Nos. WO 2006/023544, WO 2010/048352, WO2009/116037, WO 2009/147684, WO 2011/066475, WO 2011/084193, all assigned to the assignee of the instant application.

siRNA and RNA Interference

A number of PCT applications have recently been published that relate to the RNAi phenomenon. These include: PCT publication WO 00/44895; PCT publication WO 00/49035; PCT publication WO 00/63364; PCT publication WO 01/36641; PCT publication WO 01/36646; PCT publication WO 99/32619; PCT publication WO 00/44914; PCT publication WO 01/29058; and PCT publication WO 01/75164.

RNA interference (RNAi) is based on the ability of dsRNA species to enter a cytoplasmic protein complex, where it is then targeted to the complementary cellular RNA and specifically degrade it. The RNA interference response features an endonuclease complex containing a siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir et al., Genes Dev., 2001, 15(2):188-200). In more detail, longer dsRNAs are digested into short (17-29 bp) dsRNA fragments (also referred to as short inhibitory RNAs, “siRNAs”) by type III RNAses (DICER, DROSHA, etc.; Bernstein et al., Nature, 2001, 409(6818):363-6; Lee et al., Nature, 2003, 425(6956):415-9). The RISC protein complex recognizes these fragments and complementary mRNA. The whole process is culminated by endonuclease cleavage of target mRNA (McManus & Sharp, Nature Rev Genet, 2002, 3(10):737-47; Paddison & Hannon, Curr Opin Mol. Ther. 2003, 5(3):217-24). (For additional information on these terms and proposed mechanisms, see for example Bernstein et al., RNA 2001, 7(11):1509-21; Nishikura, Cell 2001, 107(4):415-8 and PCT publication WO 01/36646).

The selection and synthesis of siRNA corresponding to known genes has been widely reported; see for example Ui-Tei et al., J Biomed Biotechnol. 2006; 2006: 65052; Chalk et al., BBRC. 2004, 319(1): 264-74; Sioud & Leirdal, Met. Mol. Biol.; 2004, 252:457-69; Levenkova et al., Bioinform. 2004, 20(3):430-2; Ui-Tei et al., Nuc. Acid Res. 2004, 32(3):936-48. A siRNA is a double-stranded RNA (dsRNA) which down-regulates or silences (i.e. fully or partially inhibits) the expression of an endogenous or exogenous gene/mRNA. RNA interference is based on the ability of certain dsRNA species to enter a specific protein complex, where they are then targeted to complementary cellular RNA (i.e. mRNA), which they specifically degrade or cleave. Thus, the RNA interference response features an endonuclease complex containing siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir, et al., Genes Dev., 2001, 15:188). In more detail, longer dsRNAs are digested into short (17-29 bp) dsRNA fragments (also referred to as short inhibitory RNAs or “siRNAs”) by type III RNAses (DICER, DROSHA, etc., see Bernstein et al., Nature, 2001, 409:363-6 and Lee et al., Nature, 2003, 425:415-9). The RISC protein complex recognizes these fragments and complementary mRNA. The whole process is culminated by endonuclease cleavage of target mRNA (McManus and Sharp, Nature Rev Genet, 2002, 3:737-47; Paddison and Hannon, Curr Opin Mol. Ther. 2003, 5(3): 217-24). For additional information on these terms and proposed mechanisms, see for example, Bernstein, et al., RNA. 2001, 7(11):1509-21; Nishikura, Cell. 2001, 107(4):415-8 and PCT Publication No. WO 01/36646.

Studies have revealed that siRNA can be effective in vivo in mammals including humans. Specifically, Bitko et al., showed that specific siRNAs directed against the respiratory syncytial virus (RSV) nucleocapsid N gene are effective in treating mice when administered intranasally (Nat. Med. 2005, 11(1):50-55). For reviews of therapeutic applications of siRNAs see for example Batik (Mol. Med. 2005, 83: 764-773) and Chakraborty (Current Drug Targets 2007 8(3):469-82). In addition, clinical studies with short siRNAs that target the VEGF receptor 1 (VEGFR1) to treat age-related macular degeneration (AMD) have been conducted in human patients (Kaiser, Am J. Ophthalmol. 2006 142(4):660-8). Further information on the use of siRNA as therapeutic agents is found in Durcan, 2008. Mol. Pharma. 5(4):559-566; Kim and Rossi, 2008. BioTechniques 44:613-616; Grimm and Kay, 2007, JCI, 117(12):3633-41.

### Chemically Modified Double Stranded Nucleic Acid Molecules

The selection and synthesis of siRNA corresponding to known genes has been widely reported; (see for example Ui-Tei et al., 2006. J Biomed Biotechnol. 2006:65052; Chalk et al., 2004. BBRC. 319(1): 264-74; Sioud & Leirdal, 2004. Met. Mol. Biol. 252:457-69; Levenkova et al., 2004, Bioinform. 20(3):430-2; Ui-Tei et al., 2004. NAR 32(3):936-48).

Examples for the use of, and production of, modified siRNA are found in Braasch et al., 2003. Biochem., 42(26):7967-75; Chiu et al., 2003, RNA, 9(9):1034-48; PCT publications WO 2004/015107 (atugen AG) and WO 02/44321 (Tuschl et al). U.S. Pat. Nos. 5,898,031 and 6,107,094 teach chemically modified oligomers. U.S. Pat. No. 7,452,987 relates to oligomeric compounds having alternating unmodified and 2′ sugar modified ribonucleotides. US patent publication No. 2005/0042647 describes dsRNA compounds having chemically modified internucleoside linkages.

Amarzguioui et al., (2003, NAR, 31(2):589-595) showed that siRNA activity depended on the positioning of the 2′-O-methyl modifications. Holen et al (2003, NAR, 31(9):2401-2407) report that an siRNA having small numbers of 2′-O-methyl modified nucleosides showed good activity compared to wild type but that the activity decreased as the numbers of 2′-O-methyl modified nucleosides was increased. Chiu and Rana (2003, RNA, 9:1034-1048) teach that incorporation of 2′-O-methyl modified nucleosides in the sense or antisense strand (fully modified strands) severely reduced siRNA activity relative to unmodified siRNA. The placement of a 2′-O-methyl group at the 5′-terminus on the antisense strand was reported to severely limit activity whereas placement at the 3′-terminus of the antisense and at both termini of the sense strand was tolerated (Czauderna et al., 2003, NAR, 31(11), 2705-2716).

PCT Patent Publication Nos. WO 2008/104978 and WO 2009/044392, assigned to the assignee of the present application, and hereby incorporated by reference in their entirety, disclose motifs useful in the preparation of chemically modified siRNA compounds.

### Double Stranded RNA Oligonucleotides

In various embodiments of the methods and compositions provided herein, the nucleic acid molecule includes:

(a) a sense strand and an antisense strand;
(b) each strand of the nucleic acid molecule is independently 15 to 49 nucleotides in length;
(c) a 15 to 49 nucleotide sequence of the antisense strand is complementary to a sequence of an mRNA encoding a target gene; and
(d) a 15 to 49 nucleotide sequence of the sense strand is complementary to the antisense strand and includes a 15 to 49 nucleotide sequence of an mRNA encoding the target gene.

In various embodiments of the methods and compositions provided herein, the target genes are RTP801, REDD2 and CASP2, preferably human RTP801, human REDD2 and human CASP2 having mRNA polynucleotide sequences set forth in SEQ ID NO:1, SEQ ID NO:5, and SEQ ID NO:2-4, respectfully.

In some embodiments of the methods and compositions provided herein, the antisense strand and the sense strand of the nucleic acid molecule are independently 17-35 nucleotides in length; 17-30 nucleotides in length.; 15-25 nucleotides in length; 18-23 nucleotides in length; 19-21 nucleotides in length; 25-30 nucleotides in length; 26-28 nucleotides in length; 15-49 nucleotides in length; 15-35 nucleotides in length; is 15-25 nucleotides in length; 17-23 nucleotides in length; 17-21 nucleotides in length; 25-30 nucleotides in length; 15-25 nucleotides in length; 25-28 nucleotides in length. In some embodiments of the methods and compositions provided herein, the antisense strand and the sense strand of the nucleic acid molecule are separate polynucleotide strands.

In some embodiments of the methods and compositions provided herein, the antisense strand and the sense strand of the nucleic acid molecule are separate polynucleotide strands that form a double stranded structure by hydrogen boding. In some embodiments of the methods and compositions provided herein, the antisense strand and the sense strand of the nucleic acid molecule are separate polynucleotide strands; and wherein the antisense and sense strands are linked by covalent bonding. In some embodiments of the methods and compositions provided herein, the antisense strand and the sense strand of the nucleic acid molecule are part of a single polynucleotide strand having both a sense and antisense region. In some embodiments of the methods and compositions provided herein, the antisense strand and the sense strand of the nucleic acid molecule are part of a single polynucleotide strand having both a sense and antisense region, and wherein the nucleic acid molecule has a hairpin structure.

In various embodiments of the methods and compositions provided herein, the nucleic acid molecule comprises one or more modifications or modified nucleotides. In some embodiments of the methods and compositions provided herein, the nucleic acid molecule comprises one or more nucleotides comprising a modified sugar moiety. In some embodiments of the methods and compositions provided herein, the nucleic acid molecule comprises one or more nucleotides comprising a modified sugar; preferably a 2′-O-methyl sugar modified ribonucleotide.

In various embodiments of the methods and compositions provided herein, the nucleic acid molecule comprises one or more modified nucleobases. In various embodiments of the methods and compositions provided herein, the nucleic acid molecule comprises one or more modifications to the phosphodiester backbone. In some embodiments of the methods and compositions provided herein, the nucleic acid molecule comprises phosphodiester bonds. In some embodiments all the covalent bonds are phosphodiester bonds.

Pharmaceutical compositions disclosed herein are prepared using any chemically modified or non-modified double stranded RNA oligonucleotide compound. siRNA which are Dicer substrates or asymmetric siRNA may be used with the compositions and methods provided herein. Double stranded RNA oligonucleotide compounds used in the present disclosure encompass any pharmaceutically acceptable salts, esters, or salts of such esters, or any other compound which, upon administration to an mammal, including a human, is capable of treating diseases, disorders and injury of the CNS. The term “pharmaceutically acceptable salts” refers to physiologically and pharmaceutically acceptable salts, i.e., salts that retain the desired biological activity of the parent compound and do not impart undesired toxicological effects thereto. In some embodiments pharmaceutical compositions disclosed herein are prepared using double stranded RNA compounds that are chemically and or structurally modified according to one of the following modifications set forth in Structures disclosed herein or as tandem siRNA or RNAstar (see WO 2007/091269).

In some embodiments of a pharmaceutical composition disclosed herein, the composition comprises (a) a therapeutically effective amount of at least one oligonucleotide compound which inhibits the expression of a RTP801 gene or a REDD2 gene (b) a therapeutically effective amount of at least one oligonucleotide compound which inhibits the expression of a Casp2 gene; (c) a permeability enhancer and (d) a pharmaceutically acceptable excipient or carrier, or mixtures thereof. In some embodiments the oligonucleotide sequence of antisense strand is fully complementary to the oligonucleotide sequence of sense. In other embodiments the antisense and sense strands are substantially complementary. In certain embodiments the antisense strand is fully complementary to about 18 to about 40 consecutive ribonucleotides of the RTP801 and/or Casp2 gene. In other embodiments the antisense strand is substantially complementary to about 18 to about 40 consecutive ribonucleotides of the RTP801 and/or Casp2 gene. In some embodiments the sequence of the antisense strand is substantially complementary to from about 18 to about 40 consecutive ribonucleotides in an mRNA of the RTP801 and/or Casp2 gene associated with a disease, a disorder or an injury of the eye or CNS.

### Synthesis of Double Stranded Nucleic Acid Molecules

The double stranded nucleic acid molecules useful in preparation of the pharmaceutical compositions disclosed herein are synthesized by any of the methods that are well known in the art for synthesis of ribonucleic (or deoxyribonucleic) oligonucleotides. Such synthesis is, among others, described in Beaucage and Iyer, Tetrahedron 1992; 48:2223-2311; Beaucage and Iyer, Tetrahedron 1993; 49: 6123-6194 and Caruthers, et. al., Methods Enzymol. 1987; 154: 287-313; the synthesis of thioates is, among others, described in Eckstein, Ann. Rev. Biochem. 1985; 54: 367-402, the synthesis of RNA molecules is described in Sproat, in Humana Press 2005 edited by Herdewijn P.; Kap. 2: 17-31 and respective downstream processes are, among others, described in Pingoud et al., in IRL Press 1989 edited by Oliver R. W. A.; Kap. 7: 183-208.

Other synthetic procedures are known in the art, e.g. the procedures described in Usman et al., 1987, J. Am. Chem. Soc., 109, 7845; Scaringe et al., 1990, NAR., 18, 5433; Wincott et al., 1995, NAR. 23, 2677-2684; and Wincott et al., 1997, Methods Mol. Bio., 74, 59, may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

The oligonucleotides useful in preparation of the pharmaceutical compositions disclosed herein can be synthesized separately and joined together post-synthetically, for example, by ligation (Moore et al., 1992, Science 256, 9923; Draper et al., International Patent Publication No. WO 93/23569; Shabarova et al., 1991, NAR 19, 4247; Bellon et al., 1997, Nucleosides & Nucleotides, 16, 951; Bellon et al., 1997, Bioconjugate Chem. 8, 204), or by hybridization following synthesis and/or deprotection.

It is noted that a commercially available machine (available, inter alia, from Applied Biosystems) can be used; the oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the dsRNA or siRNA compounds disclosed herein, two or more such sequences can be synthesized and linked together for use in the present disclosure.

The double stranded RNA compounds useful in preparation of the pharmaceutical compositions disclosed herein can also be synthesized via tandem synthesis methodology, as described for example in US Patent Publication No. US 2004/0019001, wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker is selected from a polynucleotide linker or a non-nucleotide linker.

The compositions disclosed herein preferably comprise two or more oligonucleotides, these oligos may be synthesized separately and either mixed together or (covalently or non-covalently) joined together post-synthesis, or synthesized together according to the processes detailed above.

### Pharmaceutical Compositions

While it is possible for the oligonucleotide compounds to be administered as the raw chemical, it is preferable to present them as a pharmaceutical composition. In some embodiments the one or more oligoribonucleotide compounds are produced by endogenous intracellular complexes.

Disclosed herein is a pharmaceutical composition comprising one or more chemically modified double stranded RNA compounds in an amount effective to inhibit expression in a cell of a RTP801 gene, or of a REDD2 gene, and a Casp2 gene, the double stranded RNA compound comprising a sequence which is substantially complementary to the sequence of the mRNA of RTP801 (SEQ ID NO:1), or the mRNA of REDD2 (SEQ ID NO:5), and the mRNA of Casp2 gene SEQ ID NO:2-4), and a pharmaceutically acceptable carrier.

Disclosed herein is a pharmaceutical composition comprising one or more inhibitory oligonucleotide compounds; a permeability enhancer and a pharmaceutically acceptable vehicle or carrier. In some embodiments the composition comprises a mixture of two or more different oligonucleotides/siRNA compounds.

In various embodiments the penetration enhancer is selected from any compound or any combination of two ore more compounds that enhance the penetration of a therapeutic oligonucleotide through the skin and/or the tympanic membrane in the ear of a subject suffering from or at risk of a disease, a disorder or an injury of the CNS. In some embodiments the penetration/permeability enhancer is selected from, without being limited to, polyethylene glycol (PEG), glycerol (glycerin), maltitol, sorbitol etc.; diethylene glycol monoethyl ether, azone, benzalkonium chloride (ADBAC), cetylperidium chloride, cetylmethylammonium bromide, dextran sulfate, lauric acid, menthol, methoxysalicylate, oleic acid, phosphatidylcholine, polyoxyethylene, polysorbate 80, sodium glycholate, sodium lauryl sulfate, sodium salicylate, sodium taurocholate, sodium taurodeoxycholate, sulfoxides, sodium deoxycholate, sodium glycodeoxycholate, sodium taurocholate and surfactants such as sodium lauryl sulfate, laureth-9, cetylpyridinium chloride and polyoxyethylene monoalkyl ethers, benzoic acids, such as sodium salicylate and methoxy salicylate, fatty acids, such as lauric acid, oleic acid, undecanoic acid and methyl oleate, fatty alcohols, such as octanol and nonanol, laurocapram, cyclodextrins, thymol, limonene, urea, chitosan and other natural and synthetic polymers.

In certain embodiments the permeability enhancer is a polyol. In some embodiments the oligonucleotide is in admixture with a polyol. Suitable polyols for inclusion in the solutions include glycerol and sugar alcohols such as sorbitol, mannitol or xylitol, polyethylene glycol and derivatives thereof.

In some embodiments the pharmaceutical compositions disclosed herein also include one or more of various other pharmaceutically acceptable ingredients, such as, without being limited to, one ore more of buffering agent, preservative, surfactant, carrier, solvent, diluent, co-solvent, viscosity building/enhancing agent, excipient, adjuvant and vehicle. In certain embodiments accepted preservatives such as benzalkonium chloride and disodium edetate (EDTA) are included in the compositions disclosed herein in concentrations sufficient for effective antimicrobial action, about 0.0001 to 0.1%, based on the weight of the composition.

According to one embodiment the polyol is glycerol. In various embodiments glycerol is present at a final concentration of about 0.1% to about 35%; about 1% to about 30%; about 5% to about 25%, preferably about 10% to about 20% by volume of the pharmaceutical composition. In some embodiments the final concentration of glycerol in the pharmaceutical composition is about 2%, 2.5%, 5%, 10%, 12.5%, 15%, 17.5%, 20%, 22.5%, 25%, 27.5% or about 30% by volume of the pharmaceutical composition. In one embodiment, the final concentration of glycerol in the pharmaceutical composition is about 2% by volume of the pharmaceutical composition. In another embodiment, the final concentration of glycerol in the pharmaceutical composition is about 10% by volume of the pharmaceutical composition. In yet another embodiment, the final concentration of glycerol in the pharmaceutical composition is about 20% by volume of the pharmaceutical composition. In some embodiments the pharmaceutical composition is brought to about the subject's body temperature, which is about 30° C. to about 38° C., prior to application.

In various embodiments the oligonucleotide compositions are formulated for topical administration by any suitable mode of administration. Suitable modes of administration of the pharmaceutical compositions disclosed herein include invasive and non-invasive modes of administration, such as without being limited to, instillation (for example, of an eye drop solution), injection (of injectable formulation), deposition (of solid or semi-solid formulation, e.g. ointment, gel), infusion or spraying. In certain embodiments, the compositions disclosed herein are administered topically into the eye as eye drops or injected into the eye intravitreally (IVT), bilaterally or via retinal injection. Delivery can be effected by any mean (e.g. drops, spray), using any effective instrument for placing the composition inside the eye or for injecting the composition (e.g. through the vitreous humor).

In a further aspect, the present disclosure provides a method of treating a disease, a disorder or an injury of the CNS in a subject in need thereof, which comprises topically administering to the eye of the subject a pharmaceutical composition formulated as an eye drop, comprising at least one oligonucleotide directed to RTP801, or REDD2, and at least one oligonucleotide directed to Casp2, in an amount and over a period of time effective to treat the subject. In various embodiments the target mRNA is a mammalian or a non-mammalian mRNA. In some embodiments the mammalian mRNA is a human mRNA. In some embodiments the non-mammalian mRNA is a product of a gene involved in a mammalian disease, preferably human disease.

In some embodiments the pharmaceutical composition disclosed herein comprises a single type of double stranded RNA compound directed to RTP801 gene, or a single type of double stranded RNA compound directed to REDD2 gene, and a single type of double stranded RNA compound directed to Casp2 gene. In some embodiments the pharmaceutical composition disclosed herein comprises two or more different types of double stranded RNA compounds directed to RTP801 gene, or to REDD2 gene, and to Casp2 gene. In some embodiments, simultaneous inhibition of the RTP801 gene, or the REDD2 gene, and the Casp2 gene by two or more different types of double stranded RNA compounds has an additive or synergistic effect for treatment of the diseases disclosed herein.

In additional embodiments, provided herein is a pharmaceutical composition comprising a therapeutically effective amount of an oligonucleotide compound, wherein the oligonucleotide compound comprises an RTP801 dsRNA, or a REDD2 dsRNA, and a Casp2 dsRNA, further wherein the oligonucleotide compound is linked or bound (covalently or non-covalently) to an antibody or aptamer against cell surface internalizable molecules expressed on the target cells, in order to achieve enhanced targeting for treatment of the diseases disclosed herein. In various embodiments, an aptamer which acts like a ligand/antibody is combined (covalently or non-covalently) with a double stranded RNA compound in preparation of pharmaceutical compositions disclosed herein.

Also provided is a process for preparing a pharmaceutical composition disclosed herein, in accordance with formulation techniques known to those skilled in the art. In some embodiments the process for preparing a pharmaceutical composition disclosed herein, comprises combining, in any suitable order, a therapeutically effective amount of at least one oligonucleotide compound, one or more permeability enhancer and at least one pharmaceutically acceptable excipient or carrier, or mixtures thereof, such a composition preferably having extended chemical and/or physical stability as described herein. In some embodiments the process for preparing a pharmaceutical composition disclosed herein, comprises combining, in any suitable order, a therapeutically effective amount of at least one oligonucleotide compound, one or more permeability enhancer, at least one pharmaceutically acceptable excipient or carrier, or mixtures thereof and an antibacterial agent and/or preservative. In some embodiments, the pharmaceutical composition includes a pharmacologically acceptable surfactant to assist in dissolving the double stranded RNA compound. In certain embodiments a pharmaceutical composition disclosed herein further comprises an additional therapeutically active agent, such compositions being useful in combination therapies as described herein. In some embodiments of the composition disclosed herein, the additional pharmaceutically active agent, is selected from, without being limited to, non-steroidal anti-inflammatory drugs, corticosteroids, antifungal, antibiotics, and the like.

In various embodiments the pharmaceutical compositions disclosed herein comprise a therapeutically effective amount of at least one double stranded RNA compound which inhibits the expression of the RTP801 gene, or of at least one double stranded RNA compound which inhibits the expression of the REDD2 gene, and of at least one double stranded RNA compound which inhibits the expression of the Casp2 gene, preferable at least two double stranded RNA compounds, one which inhibits the RTP801 gene, or the REDD2 gene, and one which inhibits the Casp2 gene, or salt thereof, in an amount ranging from about 0.1 mg/ml to about 100 mg/ml of the composition. In some embodiments the amount of at least one double stranded RNA compound ranges from between about 1 mg/ml to about 50 mg/ml of the pharmaceutical composition. In other embodiments, the amount of at least one double stranded RNA compound ranges from between about 5 mg/ml to about 20 mg/ml of the pharmaceutical composition.

In various embodiments a pharmaceutically acceptable excipient or carrier is selected from a physiologically acceptable aqueous carrier, such as water, sodium chloride, buffer, saline (e.g. phosphate buffered saline (PBS)), mannitol, and the like, physiologically acceptable non-aqueous carrier, such as oil, and combinations thereof. Suitable aqueous and/or non-aqueous pharmaceutically acceptable carrier or vehicle is one that has no unacceptably injurious or toxic effect on the subject when administered as a component of a composition in an amount required herein. No excipient ingredient of such a carrier or vehicle reacts in a deleterious manner with another excipient or with the therapeutic oligonucleotide compound in a composition. In certain preferred embodiments the pharmaceutically acceptable carrier is water (e.g. pyrogen free water).

In another aspect, the present disclosure provides a pharmaceutical composition according to the present disclosure for treating a disease, a disorder or an injury of a peripheral nervous system and/or a central nervous system, including a visual system, and an audio-vestibular system.

### Delivery

Provided herein is a method to deliver therapeutic oligonucleotide compounds to the eye or CNS of a subject suffering from a disease, a disorder or injury of the CNS, by direct application of a pharmaceutical composition to the eye or outer ear of the subject. In some embodiments the pharmaceutical compositions disclosed herein comprise double stranded RNA compound in liposome or lipofectin formulations and the like. Such formulations can be prepared by methods well known to those skilled in the art. Such methods are described, for example, in U.S. Pat. Nos. 5,593,972, 5,589,466, and 5,580,859, which are herein incorporated by reference.

In some embodiments formulating the compositions in liposomes benefits absorption. Additionally, in some embodiments the pharmaceutical compositions comprise double stranded RNA compounds formulated with polyethylenimine (PEI), with PEI derivatives, e.g. oleic and stearic acid modified derivatives of branched PEI, with chitosan or with poly(lactic-co-glycolic acid) (PLGA). Formulating the compositions in e.g. liposomes, micro- or nano-spheres and nanoparticles, may enhance stability and benefit absorption. Examples of delivery systems useful in connection with the present disclosure include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many such implants, delivery systems, and modules are well known to those skilled in the art. In various embodiments topical non-invasive formulations are selected.

The compounds are administered as, e.g. eye drops, eye cream, eye ointment, eardrops, ear cream, ear ointment, foam, mousse, spray, solution or any of the above in combination with a delivery device. Implants of the compounds are also useful. In some embodiments liquid forms are designed for administration as eye drop or eardrops. In some embodiments liquid compositions include aqueous solutions, with and without organic co-solvents, aqueous or oil suspensions, emulsions with edible oils, as well as similar pharmaceutical vehicles. Additional formulations for improved delivery of the compounds disclosed herein can include conjugation of double stranded RNA molecules to a targeting molecule. The conjugate is usually formed through a covalent attachment of the targeting molecule to the sense strand of the double stranded RNA, so as not to disrupt silencing activity. Potential targeting molecules useful in compositions and methods disclosed herein, include proteins, peptides and aptamers, as well as natural compounds, such as e.g. cholesterol. For targeting antibodies, conjugation to a protamine fusion protein has been used (see for example: Song et al., Antibody mediated in vivo delivery of small interfering RNAs via cell-surface receptors, Nat. Biotechnol. 2005. 23(6):709-17).

In Some Embodiments the Oligonucleotides are Delivered to the Eye or CNS Tissue by Systemic Administration, Injection into Cerebrospinal Fluid (CSF), Direct Injection into the Brain, by Intravitreal Injection or by Intranasal Administration. In Other Embodiments the Oligonucleotides are Delivered by Non-Invasive Delivery Methods Such as Eye Drops or Ear Drops. ADMINISTRATION

The pharmaceutical compositions disclosed herein are administered and dosed in accordance with good medical practice, taking into account the clinical condition of the individual patient, the disease to be treated, the site and method of administration, scheduling of administration, patient age, sex, body weight and other factors known to medical practitioners.

A “therapeutically effective dose” or a “therapeutic effective amount” refers to an amount of a pharmaceutical compound or composition which is effective to achieve an improvement in a subject or his physiological systems including, but not limited to, improved survival rate, more rapid recovery, suppressed progress of the disease, or improvement or elimination of symptoms, and other indicators as are selected as appropriate determining measures by those skilled in the art.

A “therapeutically effective dose” or a “therapeutic effective amount” for purposes herein is thus determined by such considerations as are known in the art. The dose must be effective to achieve improvement including but not limited to improved survival rate or more rapid recovery, or improvement or elimination of symptoms and other indicators as are selected as appropriate measures by those skilled in the art. The pharmaceutical compositions disclosed herein are administered in a single dose or in multiple doses. It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

Pharmaceutical compositions that include the nucleic acid molecule disclosed herein may be administered once daily, qid, tid, bid, QD, or at any interval and for any duration that is medically appropriate. However, the therapeutic agent may also be dosed in dosage units containing two, three, four, five, six or more sub-doses administered at appropriate intervals throughout the day. In that case, the nucleic acid molecules contained in each sub-dose may be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. The dosage unit may contain a corresponding multiple of the daily dose.

In accordance with the disclosure provided herein is a method of treating, ameliorating, and/or slowing the progression of disease of the eye or CNS, or associated symptoms or complications thereof in a subject, the method comprising co-administering to said subject a therapeutically effective amount of at least two therapeutic agents directed to the Casp2 and RTP801 said combined administration providing the desired therapeutic effect. Combination therapy can be achieved by administering two or more agents, each of which is formulated and administered separately, or by administering two or more agents in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in some cases it is desirable that the two or more agents used in a combination therapy be present in within the patient's body at the same time, this need not be so. Combination therapy can also include two or more administrations of one or more of the agents used in the combination.

A “therapeutic combination” relates to a single composition comprising two or more therapeutic agents or to multiple compositions, each one comprising at least one therapeutic agent. As used herein, the terms “co-administer” or “co-administration” refers to administration of two or more therapeutic agents to a subject simultaneously or sequentially. The two or more therapeutic agents can be part of a single composition or separate compositions. In preferred embodiments the administration is sequential whereby the patient is treated with a Casp2 inhibitor followed by treatment with a RTP801 inhibitor or a REDD2 inhibitor.

In preferred embodiments the administration is sequential whereby the patient is treated with a RhoA inhibitor followed by treatment with a RTP801 inhibitor.

In some embodiments the agent that down regulates RTP801 expression comprises a nucleic acid molecule, preferably a dsRNA and the agent that down regulates Casp2 expression comprises a nucleic acid molecule, preferably a dsRNA. In some embodiments the agent that down regulates REDD2 expression comprises a nucleic acid molecule, preferably a dsRNA and the agent that down regulates Casp2 expression comprises a nucleic acid molecule, preferably a dsRNA. In some embodiments the agent that down regulates RTP801 expression comprises a nucleic acid molecule, preferably a dsRNA and the agent that down regulates RhoA expression comprises a nucleic acid molecule, preferably a dsRNA

In some embodiments administration of the RTP801 dsRNA or the REDD2 dsRNA is subsequent to administration of the Casp2 siRNA. In some embodiments the RTP801 dsRNA or the REDD2 dsRNA is administered to the subject between 1 minute and 60 days following administration of the Casp2 dsRNA. In some embodiments the RTP801 dsRNA or the REDD2 dsRNA is administered to the subject between 60 minutes and 60 days following administration of the Casp2 dsRNA. In some embodiments the RTP801 dsRNA or the REDD2 dsRNA is administered to the subject between 1 day and 40 days following administration of the Casp2 dsRNA; or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 days following administration of the Casp2 dsRNA. In some embodiments multiple doses of Casp2 dsRNA are administered. In some embodiments a single dose of Casp2 is administered followed by 1, 2, 3, 4, 5, 6, 7, 8, 9, 0 or more doses of RTP801 dsRNA or the REDD2 dsRNA at intervals of 10 to 30 days each administration.

Dosage is determined, inter alia, by the activity of the oligonucleotide, the indication and the severity of the disorder and comprises administering a dose of about 0.1 ng to about 50 mg, about 1 ng to about 20 mg, about 100 ng (0.1 μg) to about 20 mg, or about 10 μg to about 10 mg, or about 0.05, 0.1, 0.2, 0.3, 0.4, 0.5, 1, 2, 3, 4, 5, 6, 7, 8, 9 or 10 mg total oligonucleotide in pharmaceutically acceptable excipient or carrier. The concentration of double stranded RNA compound in the composition is between 0.1 mg/ml to 100 mg/ml, preferably between 1 mg/ml to 100 mg/ml, between 5 mg/ml to 20 mg/ml, between 10 mg/ml and 80 mg/ml or 10 mg/ml, 20 mg/ml, 30 mg/ml, 40 mg/ml, 50 mg/ml, 60 mg/ml, 70 mg/ml or 80 mg/ml. In preferred embodiments dsRNA is administered to a human eye by intravitreal injection at a dose of 0.5 mg to 30 mg per eye. Also provided are compositions, kits, containers and formulations that include a nucleic acid molecule (e.g., an siNA molecule) as provided herein for reducing expression of RTP801 or REDD2 and Casp2 for administering or distributing the nucleic acid molecule to a patient. A kit may include at least one container and at least one label. Suitable containers include, for example, bottles, vials, syringes, and test tubes. The containers can be formed from a variety of materials such as glass, metal or plastic. The container can hold the RTP801 or REDD2 and Casp2 agents which may be amino acids, small molecules, nucleic acid molecules, and/or antibody(s). Kits may further include associated indications and/or directions; reagents and other compositions or tools used for such purpose can also be included.

The container can hold a composition that is effective for treating, diagnosis, prognosing or prophylaxing a condition and can have a sterile access port (for example the container can be an intravenous solution bag or a vial having a stopper pierceable by a hypodermic injection needle). In preferred embodiments the container holds one or more nucleic acid molecule capable of specifically binding RTP801 or REDD2 and Casp2 and/or down regulating the expression of RTP801 or REDD2 and Casp2.

A kit may further include one or additional container that includes a pharmaceutically-acceptable buffer, such as phosphate-buffered saline, Ringer's solution and/or dextrose solution. It can further include other materials desirable from a commercial and user standpoint, including other buffers, diluents, filters, stirrers, needles, syringes, and/or package inserts with indications and/or instructions for use.

The units dosage ampoules or multidose containers, in which the nucleic acid molecules are packaged prior to use, may include an hermetically sealed container enclosing an amount of nucleic acid molecules or solution containing nucleic acid molecules suitable for a pharmaceutically effective dose thereof, or multiples of an effective dose. The nucleic acid molecules is packaged as a sterile formulation, and the hermetically sealed container is designed to preserve sterility of the formulation until use.

The container in which the nucleic acid molecules are packaged may include a package that is labeled, and the label may bear a notice in the form prescribed by a governmental agency, for example the Food and Drug Administration, which notice reflects approval by the agency under Federal law, of the manufacture, use, or sale of the nucleic acid molecules material therein for human administration.

In some embodiments the active dose of oligonucleotide compound for humans is in the range of from 1 ng/kg to about 20-100 mg/kg body weight per day, preferably about 0.01 mg to about 2-10 mg/kg body weight per day, in a regimen of a single dose or multiple doses administered in one dose per day or twice or three or more times per day for a period of 1-4 weeks or longer, or even for the life of the subject.

The pharmaceutical compositions of the present disclosure are administered to the subject by any suitable mode of administration. Suitable modes of administration of the oligonucleotide compositions disclosed herein include invasive and non-invasive mode of administration, such as without being limited to, instillation (of eye or ear drops), injection, deposition, or spraying into the eye or ear. In certain embodiments, the compositions disclosed herein are administered topically into the eye as eye drops or into the ear canal as ear drops or injected into the eye by intravitreal, transretinal or bilateral injection or through a cannula into the ear canal or injected through the tympanic membrane (transtympanic injection). In some embodiments the compositions disclosed herein are warmed to a temperature of about 30° C. to about 38° C. prior to administration. In many cases, the mode of administration may depend on many factors, including without being limited to, the affected eye or CNS regions, nature and severity of the disease or condition or injury being treated, as well as other clinical conditions of the individual subject.

In various embodiments the pharmaceutical compositions disclosed herein are delivered in an amount effective to provide a protective or therapeutic effect. Examples of protective or therapeutic effects include inhibition of target protein expression or knockdown of at least one target gene. In certain embodiments inhibiting expression of at least one target gene confers upon the cells and/or tissues of the CNS neuroprotective properties.

Accordingly, the pharmaceutical compositions disclosed herein are administered in any form that allows the active ingredient(s) (i.e. at least one oligonucleotide compound) to prevent, suppress, ameliorate, or otherwise treat the diseases and conditions disclosed herein. By way of non-limiting example, the pharmaceutical compositions can be formulated as a cream, foam, paste, ointment, emulsion, liquid solution, gel, spray, suspension, microemulsion, microspheres, microcapsules, nanospheres, nanoparticles, lipid vesicles, liposomes, polymeric vesicles, patches, biological inserts, aerosol, polymeric or polymeric-like material and/or any other form known in the art, including any form suitable for known or novel pharmaceutical delivery systems or devices, such as a removable and/or absorbable, dissolvable, and/or degradable implant. Sterile liquid pharmaceutical compositions, solutions or suspensions can be utilized invasively, for example, by intravitreal or transtympanic injection; or topically, e.g. by eye drop, ear drop, foam, spray, gel, cream, or ointment. The liquid compositions include aqueous solutions, with and without organic co-solvents, aqueous or oil suspensions, emulsions e.g. with edible oils, as well as similar pharmaceutical vehicles.

In various embodiments the compositions disclosed herein circumvent the blood-brain barrier (BBB) and are delivered directly to the CNS. In some embodiments the pharmaceutical composition disclosed herein is useful for delivery of the double stranded RNA compound directly into the CNS by transport along a neural pathway to the CNS, or by way of a perivascular channel, a prelymphatic channel, or a lymphatic channel associated with the brain, retina, optic nerve and/or spinal cord. In some embodiments the pharmaceutical composition disclosed herein delivers the double stranded RNA compound to the cerebrospinal fluid and then subsequently to the CNS, including the brain, retina, optic nerve and/or spinal cord.

In accordance with the method disclosed herein, the pharmaceutical compositions disclosed herein comprises one or more chemically modified double stranded RNA compounds that are delivered to the CNS by direct application of the pharmaceutical composition to the eye or outer ear.

In one embodiment, optionally used for treating patients suffering from an eye disease comprising increased ocular pressure (IOP) or NAION, a preferred dosage regimen comprises delivery of a Casp2 inhibitor, for example administering the siRNA known as QPI1007, by intravitreal injection, optionally in a single dose, followed by a series of subsequent treatments with a RTP801 inhibitor, optionally the siRNA known as PF-655, or a REDD2 inhibitor in an amount and over a period of time effective to provide neuroprotection and promote repair in the damaged neuronal retinal network of the patient.

### Methods of Treatment

A “therapeutic combination” relates to a single composition comprising two or more therapeutic agents or to multiple compositions, each one comprising at least one therapeutic agent.

In one aspect the present disclosure provides a method of treating a subject afflicted with a disease, a disorder or an injury of the eye or CNS, which comprises administering to the subject a composition comprising at least one therapeutic agent in an amount and over a period of time effective to treat the subject. In some embodiments the therapeutic agent is an oligonucleotide compound, including chemically synthesized siRNA. In additional embodiments the composition comprises at least two therapeutic agents, one of which inhibits Casp2 and one of which inhibits RTP801 or a REDD2.

In one aspect the present disclosure provides a method of treating a disease, a disorder or an injury of the eye or CNS in a subject in need thereof, which comprises administering to the eye or ear of the subject a pharmaceutical composition comprising at least one oligonucleotide compound directed to the RTP801 gene or the REDD2 gene and at least one compound directed to the Casp2 gene, in an amount and over a period of time effective to treat the subject.

In preferred embodiments the therapeutic agent is an oligonucleotide. In preferred embodiment the oligonucleotide is a siRNA compound, preferably chemically modified according to the embodiments disclosed herein. In preferred embodiments the subject being treated is a warm-blooded animal and, in particular a mammal, and preferably a human.

“Treating a subject” refers to administering to the subject a therapeutic substance effective to alleviate symptoms associated with a disease or condition, to delay the onset of the disease, to slow the progress of the disease, to lessen the severity or cure the disease, or to prevent the disease from occurring. “Treatment” refers to both therapeutic treatment and prophylactic or preventative measures, wherein the object is to prevent a disorder, to slow the progress of a disease or to reduce the symptoms of a disorder. Those in need of treatment include those already experiencing the disease or condition, those at risk of or prone to having the disease or condition, and those in which the disease or condition is to be prevented. The compositions disclosed herein are administered before, during or subsequent to the onset of the disease or condition.

The present disclosure provides a method of inhibiting the expression of RTP801 or REDD2, and Casp2 in a subject suffering from a nervous system disease, disorder or injury, which comprises administering to the subject a pharmaceutical composition comprising at least one oligonucleotide directed to the RTP801 gene or to the REDD2 gene and at least one oligonucleotide directed to the Casp2 gene, in an amount and over a period of time effective to inhibit expression of RTP801 or REDD2 and Casp2 in the nervous system of the subject.

### Combination Therapy

The methods of treating the diseases disclosed herein include administering a novel pharmaceutical composition comprising at least one chemically modified double stranded RNA compound directed to the RTP801 or REDD2 gene in conjunction or in combination with an additional inhibitor directed to the Casp2 gene, and/or a substance which improves the pharmacological properties of the chemically modified double stranded RNA compound, and/or an additional compound known to be effective in the treatment of a subject suffering from or susceptible to an eye disease, a neurodegenerative disease, a neurological disorder, a malignancy or a tumor, an affective disorder, or nerve damage resulting from a cerebrovascular disorder, injury, or infection of the CNS.

Combination therapies comprising known treatments for treating a subject suffering from or affected by or susceptible to diseases, disorders or injury of the eye or CNS, in conjunction with the novel pharmaceutical compositions and therapies described herein are considered part of the current disclosure.

In certain embodiments, the pharmaceutical compositions disclosed herein further comprise a known therapeutically active compound which is directed to treatment of eye conditions. Appropriate therapeutic amount of such a known second therapeutic agents for use in combination with a pharmaceutical composition disclosed herein are readily appreciated by those skilled in the art.

In some embodiments the combinations referred to above are presented for use in the form of a single pharmaceutical formulation.

The administration of an ocular pharmaceutical composition disclosed herein to the subject's eye is carried out by any of the many known routes of administration, including invasive and non-invasive methods of administration, as determined by a skilled practitioner. Using specialized formulations, it is possible to administer the compositions, inter alia, by instillation (e.g. of eye drops), injection, deposition, or spraying into the eye.

By “in conjunction with” or “in combination with” is meant that the additional pharmaceutically effective compound is administered prior to, at the same time as, or subsequent to administration of the pharmaceutical compositions of present disclosure. The individual components of such a combination referred to above, therefore, are administered either sequentially or simultaneously from the same or separate pharmaceutical formulations. A second therapeutic agent is administered by any suitable route, for example, by ocular, otic, oral, buccal, inhalation, sublingual, rectal, vaginal, transurethral, nasal, topical, percutaneous (i.e., transdermal), or parenteral (including intravenous, intramuscular, subcutaneous, and intracoronary) administration.

In some embodiments, an oligonucleotide disclosed herein and the second therapeutic agent/composition are administered by the same route, either provided in a single composition or as two or more different pharmaceutical compositions. However, in other embodiments, a different route of administration for the novel pharmaceutical compositions of the invention and the second therapeutic composition/agent is either possible or preferred. Persons skilled in the art are aware of the best modes of administration for each therapeutic agent, either alone or in combination.

In another aspects, the disclosure provides a pharmaceutical composition comprising two or more double stranded RNA molecules for the treatment of any of the diseases and conditions mentioned herein. In some embodiments the two or more double stranded RNA molecules or formulations comprising said molecules are admixed in the pharmaceutical composition in amounts which generate equal or otherwise beneficial activity. In certain embodiments the two or more double stranded RNA molecules are covalently or non-covalently bound, or joined together by a nucleic acid linker of a length ranging from 2-100, preferably 2-50 or 2-30 nucleotides.

In one embodiment, the two or more double stranded RNA molecules target mRNA to Casp2 and RTP801 or to Casp2 and REDD2. In some embodiments the pharmaceutical compositions disclosed herein further comprise one or more additional double stranded RNA molecule, which targets one or more additional target gene, for example a target gene disclosed in any one of PCT publications WO 2008/050329 and WO 2010/048352. In some embodiments, simultaneous inhibition of said additional gene(s) provides an additive or synergistic effect for treatment of the diseases disclosed herein.

The treatment regimen according to the disclosure is carried out, in terms of administration mode, timing of the administration, and dosage, so as to thereby treat a subject suffering from or susceptible to an eye disease, a neurodegenerative disease, a neurological disorder, a malignancy or a tumor of the CNS, an affective disorder, or nerve damage resulting from a cerebrovascular disorder, injury, or infection of the CNS.

### Indications

In various embodiments the ocular and/or otic pharmaceutical compositions disclosed herein are useful in treating or preventing various diseases, disorders and injury that affect the peripheral nervous system or the central nervous system (CNS), such as, without being limited to, the diseases, disorders and injury that are disclosed herein.

The methods and compositions provided herein has been described in an illustrative manner, and it is to be understood that the terminology used is intended to be in the nature of words of description rather than of limitation.

Many modifications and variations of the presently disclosed methods and compositions are possible in light of the above teachings. It is, therefore, to be understood that within the scope of the appended claims, the compositions and methods disclosed herein can be practiced otherwise than as specifically described.

Throughout this application, various publications, including United States Patents, are referenced by author and year and patents by number. The disclosures of these publications and patents and patent applications in their entireties are hereby incorporated by reference into this application in order to more fully describe the state of the art to which this disclosure pertains.

The present disclosure is illustrated in detail below with reference to examples, but is not to be construed as being limited thereto.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

The methods, materials and examples that will now be described are illustrative only and are not intended to be limiting; materials and methods similar or equivalent to those described herein can be used in practice or testing. Other features and advantages of the present disclosure will be apparent from the detailed description, and from the claims.

This disclosure is intended to cover any and all adaptations or variations of combination of features that are disclosed in the various embodiments herein. Although specific embodiments have been illustrated and described herein, it should be appreciated that the disclosure encompasses any arrangement of the features of these embodiments to achieve the same purpose. Combinations of the above features, to form embodiments not specifically described herein, will be apparent to those of skill in the art upon reviewing the instant description.

### EXAMPLES

Without further elaboration, it is believed that one skilled in the art can, using the preceding description, utilize the present disclosure to its fullest extent. The following preferred specific embodiments are, therefore, to be construed as merely illustrative, and not limitative of the claimed subject matter in any way.

### General Methods—Molecular Biology and Immunoassays

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out as discussed in PCR Protocols: A Guide To Methods And Applications, Academic Press, San Diego, Calif. (1990). In situ PCR in combination with Flow Cytometry (FACS) can be used for detection of cells containing specific DNA and mRNA sequences (Testoni et al., Blood 1996, 87:3822.) Methods of performing qPCR and RT-PCR are well known in the art.

Standard organic synthesis protocols known in the art not specifically described herein are generally followed essentially as in Organic syntheses: Vol. 1-79, editors vary, J. Wiley, New York, (1941-2003); Gewert et al., Organic synthesis workbook, Wiley-VCH, Weinheim (2000); Smith & March, Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

In general, ELISA is a preferred immunoassay. ELISA assays are well known to those skilled in the art. Both polyclonal and monoclonal antibodies can be used in the assays. Where appropriate other immunoassays, such as radioimmunoassays (RIA) can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, Molecular Cloning: A Laboratory Manual, Cold Springs Harbor, N.Y., 1989.

siRNA Activity

In general, about 1.5-2×10⁵ tested cells (HeLa cells and/or 293T cells for siRNA targeting human genes and NRK52 (normal rat kidney proximal tubule cells) cells and/or NMuMG cells (mouse mammary epithelial cell line) for dsRNA targeting the rat/mouse gene) are seeded per well in 6 wells plate (70-80% confluent).

About 24 hours later, cells are transfected with dsRNA compounds using the Lipofectamine™ 2000 reagent (Invitrogen) at final concentrations of 5 nM or 20 nM. The cells are incubated at 37° C. in a CO₂ incubator for 72 h. As positive control for transfection PTEN-Cy3 labeled dsRNA compounds are used. Various chemically modified dsRNA compounds are tested for activity. GFP dsRNA compounds are used as negative control for dsRNA activity. At 72 h after transfection cells are harvested and RNA is extracted from cells. Transfection efficiency is tested by fluorescent microscopy.

The percent of inhibition of gene expression using specific preferred dsRNA structures is determined using qPCR analysis of the RTP801 and/or Casp2 gene in cells expressing the endogenous gene.

In general, the dsRNAs having specific sequences that are selected for in vitro testing are specific for human and a second species such as non-human primate, rat or rabbit genes. The dsRNA compounds utilized in the studies disclosed hereinbelow are merely non-limiting examples of dsRNA that down regulate RTP801, Casp2, REDD2 and RhoA and dsRNA species having different sequences and structures that are active in down regulating their respective genes are useful in practicing the methods and kits disclosed herein. Table C provides nucleic acid sequences and SEQ ID NOS used in generating the test dsRNA compounds.

TABLE C

| | | Sense strand | | Antisense strand |
|---|---|---|---|---|
| ID | SEQ | (5′ > 3′) | SEQ | (5′ > 3′) |
| DDIT4_1 | 8 | GUGCCAACCUGAUGCAGCU | 7 | AGCUGCAUCAGGUUGGCAC |
| DDIT4_1_S500 | 10 | GUGCCAACCUGAUGCAGCU-pi | 9 | AGCUGCAUCAGGUUGGCAC-pi |
| CASP2_4 | 12 | GCCAGAAUGUGGAACUCCU | 11 | AGGAGUUCCACAUUCUGGC |
| CASP2_4_S510 | 14 | iB-GCCAGAAUGUGGAACUCcU | 13 | AGGAGUUCCACAUUCUGGC |
| DDIT4L_11 | 40 | CCUAAUGAGUGGCUAAUAA | 39 | UUAUUAGCCACUCAUUAGG |
| DDIT4L_11_S73 | 42 | CCUAAUGAGUGGCUAAUAA | 41 | UUAUUAGCCACUCAUUAGG |
| DDIT4L_6 | 44 | CCCAGAGAAUUGCUCAAGA | 43 | UCUUGAGCAAUUCUCUGGG |
| DDIT4L_6_S73 | 46 | CCCAGAGAAUUGCUCAAGA | 45 | UCUUGAGCAAUUCUCUGGG |
| EGFP_5 | 48 | GGCUACGUCCAGGAGCGCACC | 47 | GGUGCGCUCCUGGACGUAGCC |
| EGFP_5_S763 | 50 | GGCUACGUCCAGGAGCGCACC | 49 | GGUGCGCUCCUGGACGUAGCC |
| RHOA_29 | 52 | UCGACAGCCCUGAUAGUUU | 51 | AAACUAUCAGGGCUGUCGA |
| RHOA_29_S73 | 54 | UCGACAGCCCUGAUAGUUU | 53 | AAACUAUCAGGGCUGUCGA |
| siCTR | 56 | [symbol] | 55 | GCAUCGCGCGUAAUUUAGU |

Table C Legend: Capital letters: unmodified ribonucleotide; underlined capital letter: 2′O-methyl sugar modified ribonucleotide, lower case underlined letter: L-deoxynucleotide; pi-phosphorylated ribonucleotide [symbol]
indicates data missing or illegible when filed

### Serum Stability Experiments

Chemically modified dsRNA compounds according to the present disclosure are tested for duplex stability in human serum, as follows:

dsRNA molecules at final concentration of 7 uM are incubated at 37° C. in 100% human serum (Sigma Cat#H4522). (dsRNA stock 100 uM diluted in human serum 1:14.29).

Typically 5 ul are added to 15 ul 1.5xTBE-loading buffer at different time points (0, 30 min, 1 h, 3 h, 6 h, 8 h, 10 h, 16 h and 24 h). Samples are immediately frozen in liquid nitrogen and were kept at −20° C.

Each sample is loaded onto a non-denaturing 20% acrylamide gel, prepared according to methods known in the art. The samples are visualized with ethidium bromide under UV light.

### Example 1

### Synergistic Effect of Combined RTP801 and Caspase2 Inhibition

Several dsRNA molecules targeting RTP801 have been generated and tested according to, without being limited to, the methods disclosed in PCT publications WO 2006/023544, WO 2007/084684, WO 2008/106102 and WO2009/116037. Some dsRNA molecules targeting Casp2 have been tested according to the methods disclosed in PCT publications WO 2006/035434, WO 2010/048352 and WO 2011/072091. Combination therapy utilizing dsRNA to RTP801 (“siRTP801”) and dsRNA to Casp2 (“siCasp2”) was tested as follows:

The Casp2 dsRNA compound (Casp2 siRNA, designated as “siCasp2” or “CASP2_{—}4_S510 siRNA” or “QPI1007”) that was used in the preparation of the pharmaceutical composition utilized in this study is a proprietary 19-mer blunt-ended duplex having two separate strands, with an antisense strand (AS, guide strand) comprising unmodified ribonucleotides at positions 1, 3, 5, 7, 9, 10, 12, 14, 16 and 18 (capital letters), and 2′OMe sugar modified ribonucleotides at positions 2, 4, 6, 8, 11, 13, 15, 17 and 19 (lower case letters); and a sense strand (SEN, passenger strand) comprising unmodified ribonucleotides and an L-deoxyribonucleotide at position 18 (underlined lower case) and an inverted deoxyabasic moiety (iB) at the 5′ terminus, as depicted:

(antisense strand, SEQ ID NO: 11)
5′ AGGAGUUCCACAUUCUGGC 3′
(sense strand, SEQ ID NO: 12)
3′ UcCUCAAGGUGUAAGACCG-iB 5′

The RTP801 dsRNA compound (designated as “REDD14”, “DDIT4 siRNA”, ‘PF-655” or “PF-04523655”) that was used in the preparation of the composition utilized in this study is a 19-mer blunt-ended duplex having two separate strands, with an antisense strand (AS, guide strand) comprising 2′OMe sugar modified ribonucleotides at positions 1, 3, 5, 7, 9, 11, 13, 15, 17 and 19 (lower case letters), unmodified modified ribonucleotides at positions 2, 4, 6, 8, 10, 12, 14, 16 and 18 (capital letters), and a sense strand (SEN, passenger strand) comprising unmodified ribonucleotides at positions 1, 3, 5, 7, 9, 11, 13, 15, 17 and 19 (capital letters) and 2′OMe sugar modified ribonucleotides at positions 2, 4, 6, 8, 10, 12, 14, 16 and 18 (lower case letters), set forth in SEQ ID NOS:9 and 10, respectively.

The dsRNA to RhoA comprises alternating 2′-OMe sugar modified ribonucleotides and unmodified ribonucleotides in both the sense strand and antisense strand. The pattern of alternating modified and unmodified nucleotides starts with a modified nucleotide at the 5′ end of the antisense strand and the terminal nucleotide at the 3′ end is also a 2′-O-Methyl sugar modified ribonucleotide. In the sense strand the terminal nucleotides at the 5′ end and at the 3′ end are unmodified ribonucleotides and the penultimate nucleotides at the 5′ end and at the 3′ end are 2′-O-Methyl sugar modified ribonucleotides, set forth in SEQ ID NOS:53 and 54, respectively Thus, the pattern of modified nucleotides is configured such that modified nucleotides in the sense strand are opposite unmodified nucleotides in the antisense strand and vice-versa.

Experimental evidence of advantages for combined use of a Casp2 inhibitor (e.g. dsRNA targeting Casp2) and a RTP801 inhibitor (e.g. dsRNA targeting RTP801) for promoting retinal ganglion cell (RGC) survival:

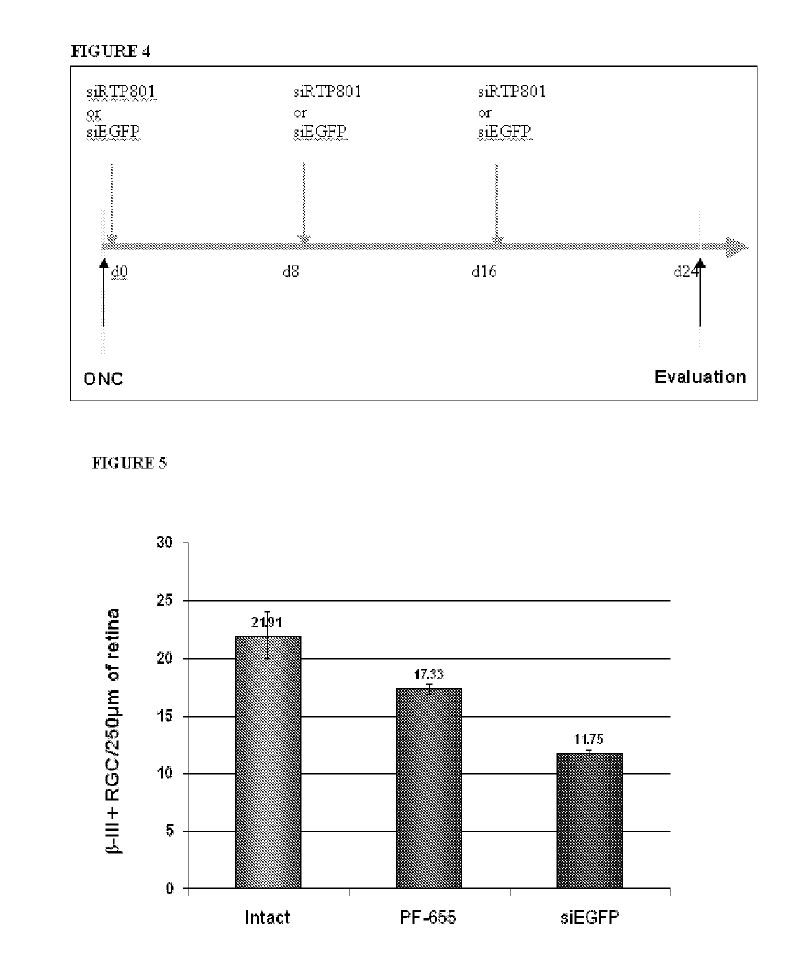

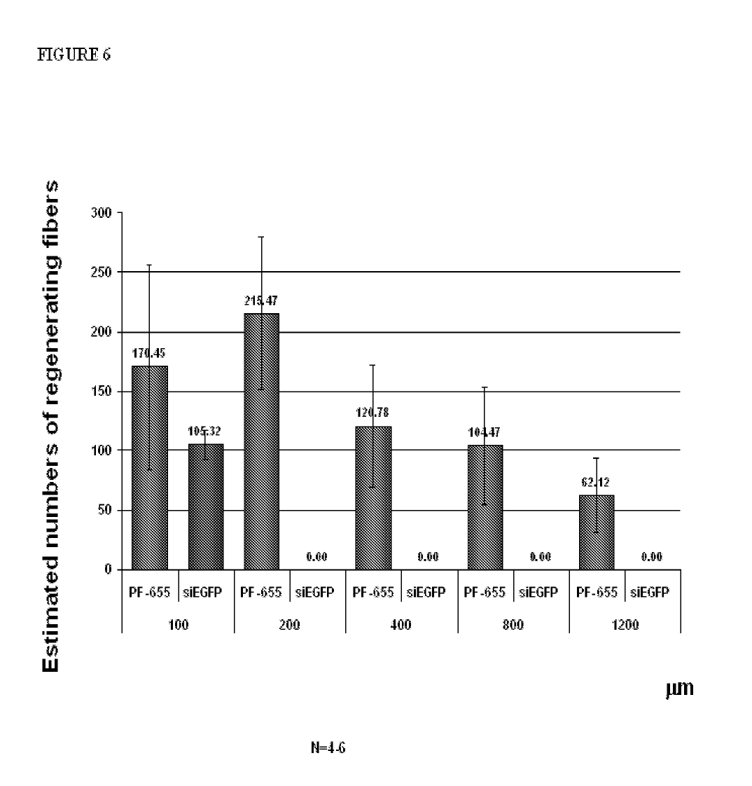

In-vivo study design: Wistar rats received a bilateral optic nerve crush (ONC) injury that transected 100% RGC axons on day 0, this was immediately followed by an intravitreal injection of either PBS (vehicle), “siEGFP” (EGFP inhibitor; negative control), or the following combinatorial therapies: “PF-04523655” (RTP801 inhibitor)+“QPI1007” (Casp2 inhibitor), “PF-04523655”+“siEGFP” or “QPI1007”+“siEGFP”. Animals received additional dsRNA injections on days 8 and 16 and, on day 24, eyes and optic nerves were harvested and prepared for histological examination (FIG. 4). Retinal sections were stained for βIII-tubulin to evaluate RGC survival. For siEGFP in monotherapy the animals were injected with 40 ug/eye. Within combinations, each individual dsRNAs was injected at 20 ug/eye, so that total amount of injected dsRNA was 20 ug/eye+20 ug/eye=40 ug/eye.

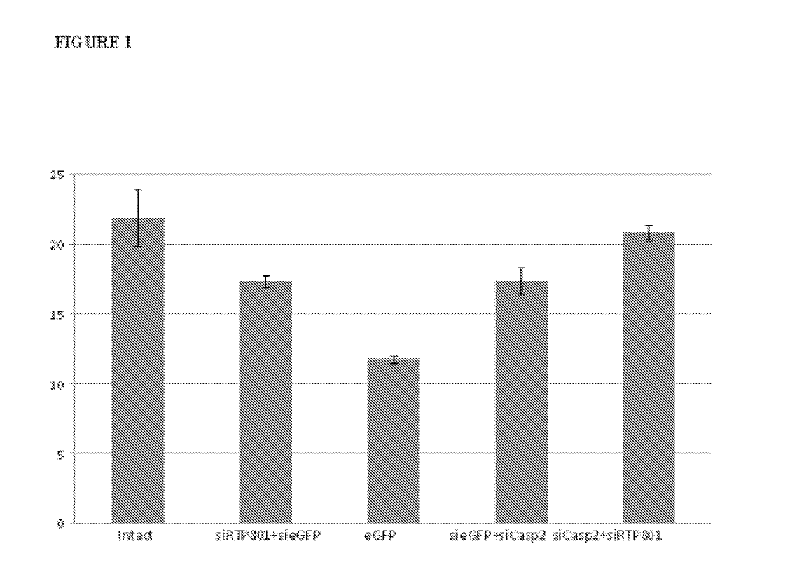

Results: Treatment with “QPI1007”+“siEGFP” or “PF-04523655”+“siEGFP” significantly increased RGC survival compared to siEGFP and PBS treatments (FIG. 1). FIG. 5 also shows the level of neuroprotection (RGC survival) achieved in “PF-04523655”+“siEGFP” treated eyes. The neuroprotective effects of a RTP801 inhibitor was enhanced by the combinatorial administration of Casp2 inhibitor (“QPI1007”) and RTP801 inhibiroe (identified as “PF-04523655” or as “PF-655’). FIG. 1 shows that when given together, “PF-04523655” and “QPI1007” caused a significant increase in RGC survival (˜95%) compared to “QPI1007” and “PF-04523655” mono-therapies (˜79%).

Experimental evidence of advantages for use of a RTP801 inhibitor (dsRNA targeting RTP801) for promoting axon outgrowth:

In-vivo Study design: was as described hereinabove in the “Experimental evidence of advantages of combined use of a Caspase2 inhibitor (dsRNA targeting Casp2) and a RTP801 inhibitor (ds RNA targeting RTP801) for promoting retinal ganglion cell (RGC) survival” section.

Optic nerves were immunostained for growth associated protein-43 (GAP-43) to assess RGC axonal regeneration. Axon growth was quantified according to methodology described in (Leon S, Yin Y, Nguyen J, Irwin N, Benowitz L I. Lens injury stimulates axon regeneration in the mature rat optic nerve. J. Neurosci. 2000 Jun. 15; 20(12):4615-26).

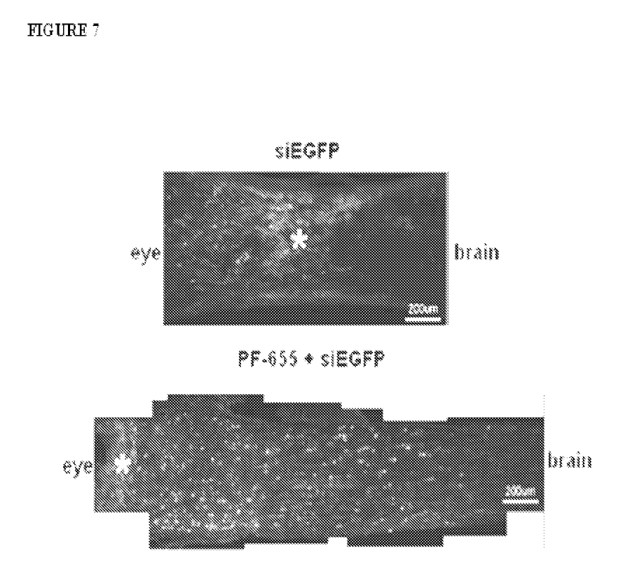

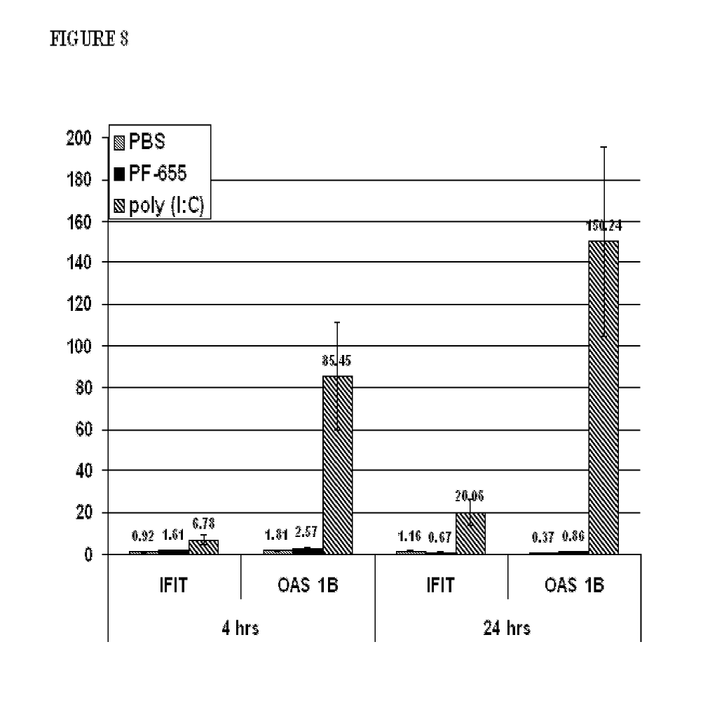

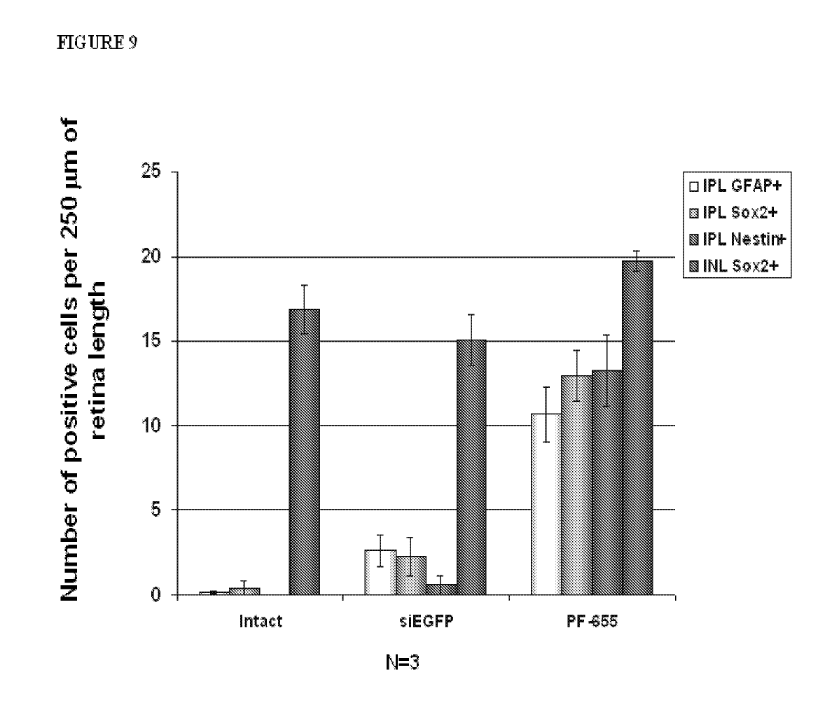

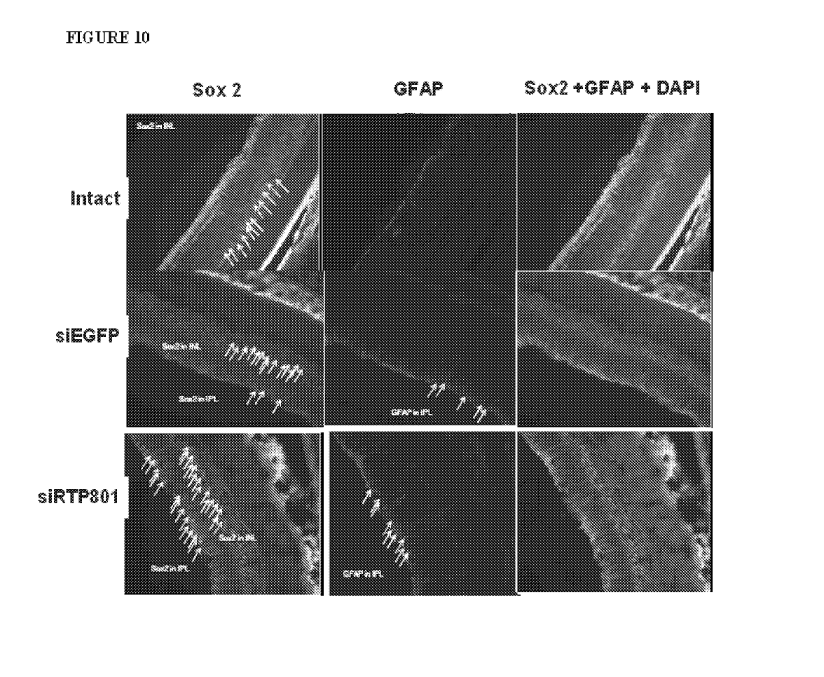

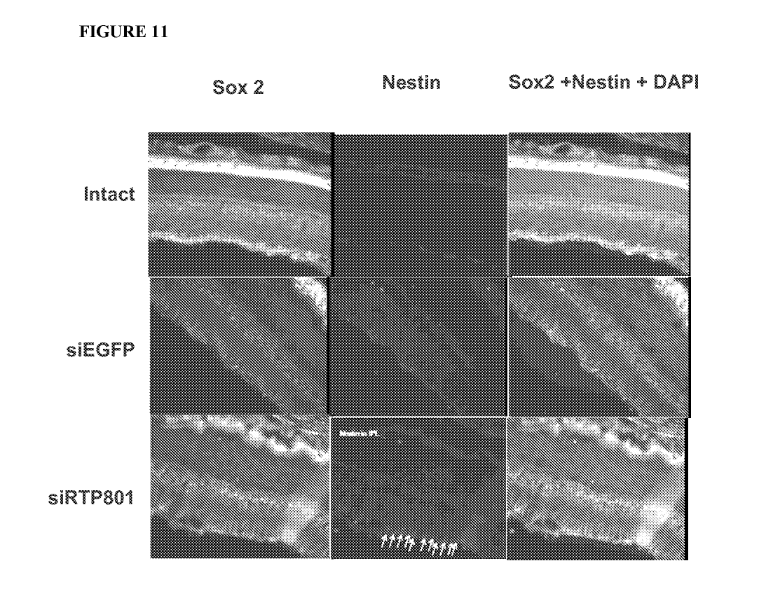

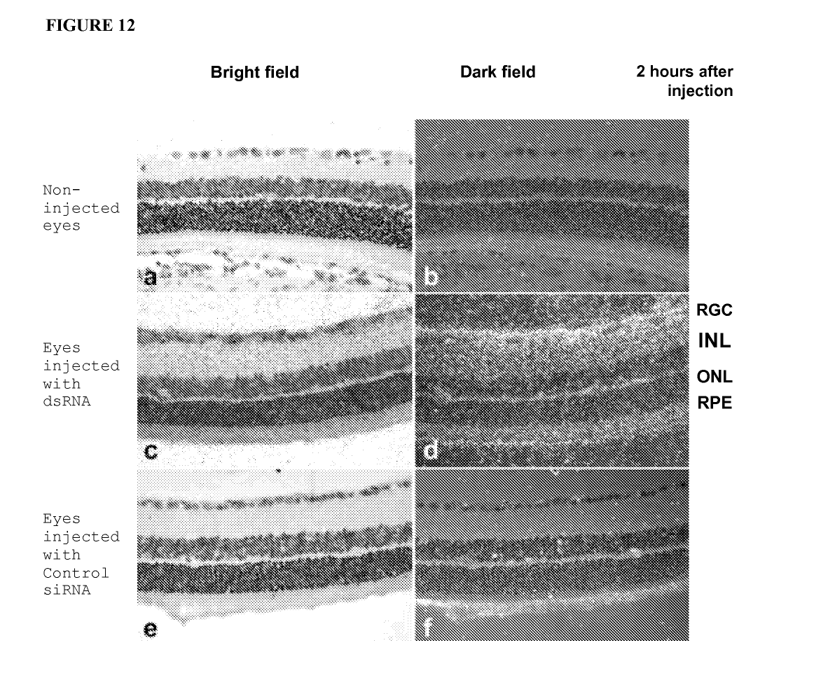

Results: Treatment with “PF-04523655” (RTP801 inhibitor) promoted significant RGC axonal regeneration into the distal stump of the optic nerve (for distances of up to 1200 μm), irrespective of whether it was administered alone or in combination with QPI1007 (FIG. 2). The effect of monotherapy with an RTP801 inhibitor is also shown in FIGS. 6 and 7. The effect was RTP801-specific (control siEGFP was not axogenic).

### Example 2

### Rat Model of Glaucoma and of Retinal Ganglion Cells (RGC) Death

ONC model in rats: Various animal models are useful for studying the effect of siRNA therapeutics in treating glaucoma. In the optic nerve crush (ONC) model in rats the orbital optic nerve (ON) of anesthetized rats is exposed through a supraorbital approach, the meninges severed and all axons in the ON transected by crushing with forceps for 10 seconds, 2 mm from the lamina cribrosa. Testing pharmaceutical compositions disclosed herein (comprising at least one inhibitor, preferably a dsRNA compound directed at the RTP801 gene, or the REDD2 gene, and a dsRNA compound directed at the Casp2 gene, preferably at least two dsRNAs, at least one targeting RTP801 or REDD2, and at least one targeting Casp2 for treating or preventing glaucoma is performed, for example, in the animal models described by Pease et al. (J. Glaucoma, 2006, 15(6):512-9. Manometric calibration and comparison of TonoLab and TonoPen tonometers in rats with experimental glaucoma and in normal mice).

Optic nerve crush (ONC) model in adult Wistar rats is also an accepted model for studying Retinal Ganglion Cells (RGC) death. The onset and kinetics of RGC death in this model are very reproducible; RGC apoptosis begins on day 4-5 after the ONC; massive RGC loss (about 50-60%) is observed on days 7-10 after the ONC; and 95% of the RGC loss is occurs by week 3-4 after the ONC. This model allows for establishment of the neuroprotective efficacy of test drugs in vivo.

Pharmaceutical compositions comprising a RTP801 inhibitor and a Casp2 inhibitor are tested in this animal model, which shows that these compositions treat and/or prevent glaucoma and/or RGC death.

### Example 3

### Animal Models for Testing dsRNA Compounds in Spinal Cord Injury

In a non-limiting example, testing of the compositions disclosed herein comprising dsRNA inhibitors for treating spinal cord injury is performed in the rat spinal cord contusion model as described by Young (Prog Brain Res. 2002; 137:231-55). Other predictive animal models of spinal cord injury are described in the following references: Gruner J A. “A monitored contusion model of spinal cord injury in the rat.” J. Neurotrauma 1992. 9(2): 123-128; Hasegawa K and Grumet M. “Trauma-induced tumorigenesis of cells implanted into the rat spinal cord.” J. Neurosurg. 2003. 98(5): 1065-71; and Huang P P and Young W. “The effects of arterial blood gas values on lesion volumes in a graded rat spinal cord contusion model.” J Neurotrauma 1994, 11(5): 547-562.

Pharmaceutical compositions comprising a RTP801 inhibitor and a Casp2 inhibitor are tested in this animal model, which shows that these compositions treat spinal cord injury.

### Example 4

### Rat Models for Testing the dsRNA Compounds in CNS Injury

Closed Head Injury (CHI): Experimental traumatic brain injury (TBI) produces a series of events contributing to neurological and neurometabolic cascades, which are related to the degree and extent of behavioral deficits. CHI is induced under anesthesia, while a weight is allowed to free-fall from a prefixed height (Chen Y et al, J. Neurotrauma. 1996; 13:557-568) over the exposed skull covering the left hemisphere in the midcoronal plane.

Transient middle cerebral artery occlusion (MCAO): A 90 to 120 minutes transient focal ischemia is performed in adult, male Sprague Dawley rats, 300-370 gr. The method employed is the intraluminal suture MCAO (Longa E Z et al., Stroke 1989, 20, 84-91, and Dogan A. et al., J. Neurochem. 1999, 72, 765-770). Briefly, under halothane anesthesia, a 3-O-nylon suture material coated with Poly-L-Lysine is inserted into the right internal carotid artery (ICA) through a hole in the external carotid artery. The nylon thread is pushed into the ICA to the right MCA origin (20-23 mm). 90-120 minutes later the thread is pulled off, the animal is closed and allowed to recover.

Permanent middle cerebral artery occlusion (MCAO): Occlusion is permanent, unilaterally-induced by electrocoagulation of MCA. Both methods lead to focal brain ischemia of the ipsilateral side of the brain cortex leaving the contralateral side intact (control). The left MCA is exposed via a temporal craniotomy, as described for rats by Tamura A. et al., J Cereb Blood Flow Metab. 1981; 1:53-60. The MCA and its lenticulostriatal branch are occluded proximally to the medial border of the olfactory tract with microbipolar coagulation. The wound is sutured, and animals returned to their home cage in a room warmed at 26° C. to 28° C. The temperature of the animals is maintained all the time with an automatic thermostat.

Evaluation Process: The efficacy of the pharmaceutical compositions disclosed herein for treating CNS injury is determined by mortality rate, weight gain, infarct volume, short and long term clinical, neurophysiological and behavioral (including feeding behavior) outcomes in surviving animals. Infarct volumes are assessed histologically (Knight R A et al., Stroke. 1994, 25, 1252-1261 and Mintorovitch J. et al., Magn. Reson. Med. 1991. 18, 39-50). The staircase test (Montoya C P et al., J. Neurosci. Methods 1991, 36, 219-228) or the motor disability scale according to Bederson's method (Bederson J B et al., Stroke, 1986, 17, 472-476) is employed to evaluate the functional outcome following MCAO. The animals are followed for different time points, the longest one being two months. At each time point (24 hours, 1 week, 3, 6, 8 weeks), animals are sacrificed and cardiac perfusion with 4% formaldehyde in PBS is performed. Brains are removed and serial coronal 200 μm sections are prepared for processing and paraffin embedding. The sections are stained with suitable dyes such as TCC. The infarct area is measured in these sections using a computerized image analyzer.

Pharmaceutical compositions comprising a RTP801 inhibitor and a Casp2 inhibitor are tested in this animal model, which shows that these compositions treat and/or prevent CNS injury.

### Example 5

### APP Transgenic Mouse Model of Alzheimer's Disease

Animals and Treatment: The study includes twenty-four (24) APP^{V717I} transgenic mice (female), a model for Alzheimer's disease (Moechars D. et al., EMBO J. 1996, 15(6):1265-74; and Moechars D. et al., Neuroscience. 1999, 91(3):819-30), aged 11 months that are randomly divided into two equal groups (Group I and Group II).

Animals are treated with a pharmaceutical composition comprising at least one siRNA compound directed at the RTP801 and/or Casp2 gene. Animals in control groups are treated with a vehicle solution. Compositions comprising the following concentrations of siRNA are tested: (i) 100 μg of siRNA compound/3 μl of vehicle; (ii) 200 μg of siRNA compound/3 μl of vehicle and (iii) 500 μg of siRNA compound/3 μl of vehicle. Compositions comprising the following vehicle are tested: (i) 5% glycerol solution; (ii) 10% glycerol solution and (iii) 15% glycerol solution. In this study the compositions are administered once every 4 days, during 3-4 month period of the experiment.

Termination: Mice are sacrificed; brains are dissected and processed as follows: one hemisphere for histological analysis and one hemisphere for molecular biology analysis.

Evaluation Process: The following histological analysis is performed:

1. Anti-amyloid β (Aβ) staining and quantification (4 slides/mouse)
2. Thioflavin S staining and quantification of Aβ plaques (4 slides/mouse)
3. CD45 staining and quantification (4 slides/mouse)
4. GFAP (astrocytosis) staining and quantification

Results: Pharmaceutical compositions comprising a RTP801 inhibitor and a Casp2 inhibitor are tested in this animal model, which shows that these composition are useful in treating Alzheimer's disease.

### Example 6

### Mouse Model of ALS

Objective: To examine the efficacy of pharmaceutical composition comprising an RTP801 siRNA and a Casp2 siRNA in the mutant SOD1^{G93A} mouse model of ALS.

Animals and Treatment: The following experimental groups are used for studying disease progression and lifespan:

Group 1 (Test)—is administered with an composition comprising at least one siRNA compound downregulating RTP801 and at least one siRNA compound down-regulating Casp2 gene. This group comprises wild-type (n=10) and SOD1^{G93A} (n=10) mice.
Group 2—(Control siRNA): is administered with an composition comprising a control siRNA compound (such as EGFP siRNA). This group comprises wild-type (n=10) and SOD1^{G93A} (n=10) mice.
Group 3 (Vehicle): is administered with a vehicle solution (such as 10% glycerol solution). This group comprises wild-type (n=10) and SOD1^{G93A} (n=10) mice.
Group 4 (Untreated controls)—This group comprises wild-type (n=10) and SOD1^{G93A} (n=10) mice.

Each experimental group is sex matched (5 male, 5 female) and contain littermates from at least 3 different litters. This design reduces bias that may be introduced by using mice from only a small number of litters, or groups of mice with a larger percentage of female SOD1^{G93A} mice (since these mice live 3-4 days longer than males).

Animals in test group are treated with pharmaceutical composition comprising at least one siRNA compound directed at the RTP801 gene and at least one dsRNA compound directed at the Casp2 gene. Animals in control dsRNA group are treated with a composition comprising a control dsRNA compound (dsRNA targeting EGFP). Animals in vehicle group are treated with a vehicle solution. Compositions comprising the following concentrations of dsRNA are tested: (i) 100 μg of dsRNA compound/3 μl of vehicle; (ii) 200 μg of dsRNA compound/3 μl of vehicle and (iii) 500 μg of dsRNA compound/3 μl of vehicle. In this study the compositions are administered once every 4 days, starting from 30 days of age.

Analysis of disease progression: Behavioral and electromyography (EMG) analysis in treated and untreated mice is performed to monitor disease onset and progression. Mice are pre-tested before start of treatment, followed by weekly assessments. All results are compared statistically. The following tests are performed:

1. Swimming tank test: this test is particularly sensitive at detecting changes in hind-limb motor function (Raoul C et al., Nature Med. 2005. 11, 423-428; Towne C et al, Mol. Ther. 2008, 16:1018-1025).
2. Electromyography: EMG assessments are performed in the gastrocnemius muscle of the hind limbs, where compound muscle action potential (CMAP) is recorded (Raoul C et al., 2005. supra).
3. Body weight: The body weight of mice is recorded weekly, as there is a significant reduction in the body weight of SOD1^{G93A} mice during disease progression (Kieran D et al., PNAS, 2007, 104(51): 20606-20611).

Assessment of lifespan: The lifespan in days for treated and untreated mice is recorded and compared statistically to determine whether treatment by administering a pharmaceutical composition comprising dsRNA directed at the RTP801 and Casp2 gene implicated in ALS has any significant effect on lifespan. Mice are sacrificed at a well-defined disease end point, when they have lost >20% of body weight and are unable to raise themselves in under 20 seconds. All results are compared statistically.

Post mortem histopathology: At the disease end-point mice are terminally anaesthetized and spinal cord and hind-limb muscle tissue are collected for histological and biochemical analysis.

Examining motor neuron survival: Transverse sections of lumbar spinal cord are cut using a cryostat and stained with gallocyanin, a nissl stain. From these sections the number of motor neurons in the lumbar spinal cord is counted (Kieran et al., 2007. supra), to determine whether dsRNA treatment prevents motor neuron degeneration in SOD1^{G93A} mice.

Examining spinal cord histopathology: Motor neuron degeneration in SOD1^{G93A} mice results in astrogliosis and activation of microglial cells. Here, using transverse sections of lumbar spinal cord the activation of astrocytes and microglial cells is examined using immunocytochemistry to determine whether siRNA treatment reduced or prevented their activation.

Examining muscle histology. Hind-limb muscle denervation and atrophy occur as a consequence of motor neuron degeneration in SOD1^{G93A} mice. At the disease end point the weight of individual hind-limb muscles (gastrocnemius, soleus, tibialis anterior, extensor digitorium longus muscles) is recorded and compared between treated and untreated mice. Muscles are then processed histologically to examine motor end plate denervation and muscle atrophy (Kieran et al., 2005. J. Cell Biol. 169, 561-567).

### Example 7

### Model Systems of Huntington's Disease (HD) in Mice

In a non-limiting example, testing of the compositions disclosed herein for treating Huntington's disease is performed in the HD mouse model, R6/2 as described by Yu-Lai Wang et al. (Clinico-pathological rescue of a model mouse of Huntington's disease by siRNA. Neurosci Res 53(3):241-9).

Results: The compositions and methods disclosed herein are tested in this animal model, and show efficacy in treating Huntington's disease.

### Example 8

### The Effect of a RTP801 Inhibitor on Gene Expression in RPE and Neural Retina in Laser-Induced CNV Model in Mice

This experiment was designed in order to study the effect of RTP801 dsRNA inhibitor (“REDD14”) on gene expression in RPE and neural retina.

### Experimental Design

### Groups:

PBS (one eye of each animal)
“REDD14” 0.25 mg (another eye of each animal)

The study was performed in 5 mice per time point. The treatment Group size was 5 eyes. CNV was induced by laser treatment as described hereinbelow; “REDD14” (the dsRNA compound targeting RTP801) was injected immediately after CNV induction on day zero. The effect on gene expression was evaluated by qPCR analysis in RPE and neural retina on day 5 after CNV induction and compared to expression of the same genes in non-treated eyes (n=5).

Preparation of Test and Control Articles: For injection, test article (REDD14 dsRNA) was diluted from stock solution to the appropriate concentrations of the necessary dose to 2 μl volume using sterile PBS for injections.

### Equipment: Laser (OcuLight GL, IRIDEX, Mountain View, Calif.)

### Experimental Procedures

Anesthesia: For all procedures, anesthesia was achieved by i.p. injection of 50 mg/kg mouse body weight of Ketaject (Phoenix Pharmaceutical, Inc., St. Joseph, Mo.) and 10 mg/kg xylazine (The Butler Company, Dublin, Ohio), and pupils were dilated with topical 1% tropicamide (Bausch & Lomb, Tampa, Fla.).

Intravitreous Injections: All intravitreal injections were performed using 33-gauge needles. The injected volumes were 2 μl. The injection of test and control articles were performed on days 0 immediately after photocoagulation and 1 week later.

CNV Induction: Choroidal neovascularization (CNV) was triggered by laser photocoagulation (532 nm, 200 mW, 75 μm) (OcuLight GL, IRIDEX Corp.) performed on both eyes of each mouse on day 0 by a single individual masked to drug group assignment. Laser spots were applied in a standardized fashion around the optic nerve (3-4 spots/eye), using a slit lamp delivery system and a cover slip as a contact lens.

Results:

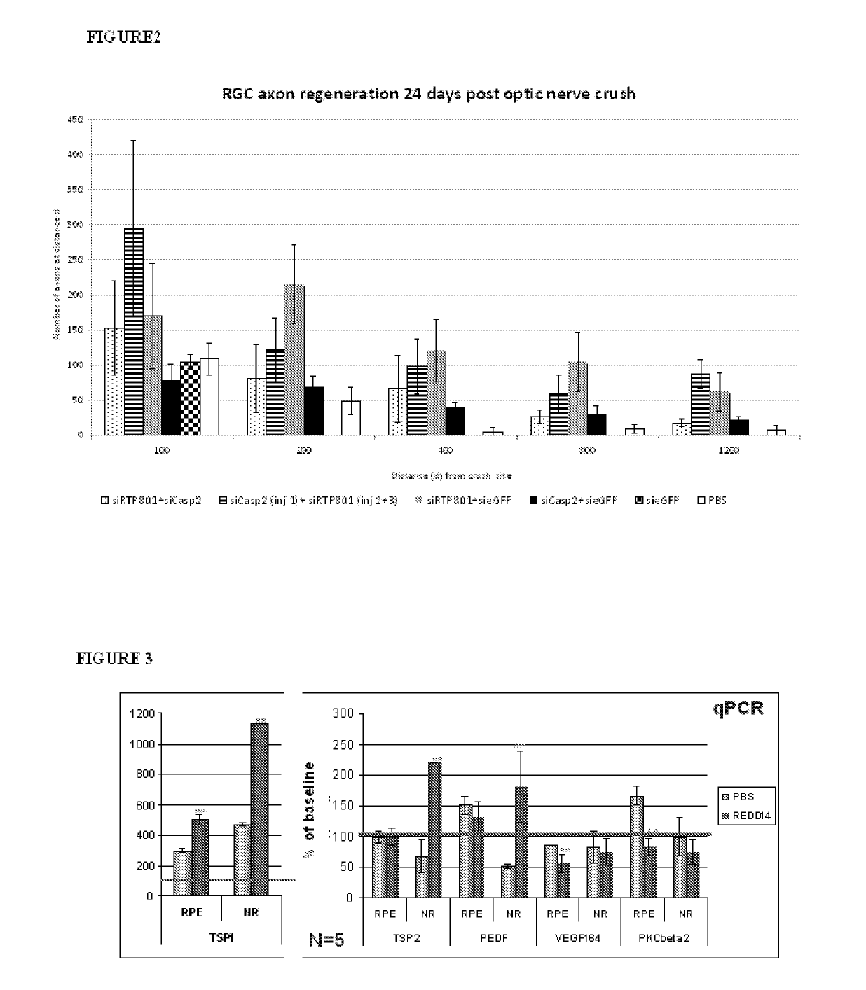

The results of the above experiment are presented in FIG. 3. FIG. 3 shows that inhibition of RTP801 expression by dsRNA targeting RTP801 in mouse eyes induces expression of PEDF (antiangiogenic and neuroprotective factor; described for example in Miyazaki M et al. (2011) Hum Gene Ther. May; 22(5):559-65) and thrombospondin 1 and 2 (extracellular matrix components critically important for formation of neural connections; described for example in Neugebauer K M et al. (1991) Neuron. March; 6(3):345-58).

These results show that the administration of REDD14 causes:
˜70% upregulation of PEDF expression over the baseline in neural retina (note: in PBS-injected eyes expression of PEDF is 40% down regulated below the baseline);
˜40% down regulation of VEGF164 expression below the baseline in RPE (note: in PBS-injected eyes, expression of VEGF164 is 20% down regulated);
˜1000% upregulation of TSP1 expression over the baseline in neural retina (note: in PBS-injected eyes expression of TSP1 is ˜400% upregulated over the baseline);
˜400% upregulation of TSP1 expression over the baseline in RPE (note: in PBS-injected eyes expression of TSP1 is ˜200% upregulated over the baseline);
˜125% upregulation of TSP2 expression over the baseline in neural retina (note: in PBS-injected eyes expression of TSP2 is 60% down regulated below the baseline).

### Example 9

### Evaluation of Protection of Retinal Ganglion Cell Dendrites after Axonal Injury in RGC-YFP Transgenic Mice Model by Intravitreally Injected dsRNAs Targeting REDD2, RTP801 or Casp2 or Combined Treatment with dsRNAs Targeting RTP801 and Casp2

Experimental animals and surgical procedure: Experimental procedures were carried out on C57BL/6 transgenic or wild-type control mice. Adult transgenic mice carrying the yellow fluorescent protein (YFP) gene under control of the Thy-1 promoter (YFP-H line, Jackson Laboratory, Bar Harbor, Me., USA;) were studied (Feng et al. (2000) al Approximately 10-30% of retinal ganglion cells are exclusively labelled in the retina of these transgenic mice. All surgical procedures were carried out on 3 to 7 month-old mice under general anesthesia (2% Isoflurane; 0.8 L/min).

Optic nerve axotomy: The optic nerve axotomy was carried out on mice as previously described (Lebrun-Julien et al., 2009). Briefly, the left optic nerve was exposed and carefully transected at 0.5-1 mm from the optic nerve head. During this procedure care was taken to avoiding injury to the ophthalmic artery. Fundus examination was routinely performed immediately after axotomy and 3 days later to verify the integrity of the retinal circulation after surgery. Animals showing signs of compromised blood supply were excluded from the study.

Intravitreal injection: Double stranded RNA compounds were used in this study to modulate the activation of mTOR signalling pathways. Single intravitreal injection (2 μl) was made into the vitreous chamber of the left eye of YFP mice at the time of the optic nerve injury. The intravitreal injections were made using a 10 μl Hamilton syringe adapted with a 32 gauge glass micro needle as described previously (Lebrun-Julien et al., 2009). Briefly, the micro needle was introduced in the superior hemisphere of the ocular globe. During this procedure care was taken to avoiding lens injury by introducing the micro needle at an angle of 45 degree through the sclera. The injection was performed over a period of 2 minutes and the needle was held still during another 2 minutes to enable the siRNA to diffuse into the vitreous chamber. After the injection, surgical glue (Indermill, Tyco Health Care, Mansfield, Mass., USA) was immediately used to seal the site of injection, avoiding any leakage.

All dsRNA molecules used in this study were chemically stabilized and were synthesized at BioSpring (Frankfurt, Germany). dsRNA compounds targeting REDD2 (“DDIT4L_{—}11_S73”), EGFP (“EGFP_{—}5_S763”) and a control dsRNA compound (“CNL_{—}1_S73”) that were used in this experiment had the sequence shown in Table C, supra.

The dsRNA compounds targeting REDD2 (“DDIT4L_{—}11_S73”), EGFP (“EGFP_{—}5_S763”) and a control dsRNA compound (“CNL_{—}1_S73”) used in this experiment were 19-mer blunt-ended duplexes having two separate strands, with an antisense strand comprising 2′OMe sugar modified ribonucleotides at positions 1, 3, 5, 7, 9, 11, 13, 15, 17 and 19 (capital bold underlined letters), unmodified modified ribonucleotides at positions 2, 4, 6, 8, 10, 12, 14, 16 and 18 (capital letters), and a sense strand (SEN, passenger strand) comprising unmodified ribonucleotides at positions 1, 3, 5, 7, 9, 11, 13, 15, 17 and 19 (capital letters) and 2′OMe sugar modified ribonucleotides at positions 2, 4, 6, 8, 10, 12, 14, 16 and 18 (capital bold underlined letters), shown in Table C hereinabove.

The Casp2 dsRNA compound (Casp2 siRNA, designated as “siCasp2” or “Casp2_{—}4_S510 siRNA” or “QPI1007”) used in the preparation of the pharmaceutical composition utilized in this study is a proprietary 19-mer duplex having two separate strands, with an antisense strand (AS, guide strand) comprising unmodified ribonucleotides at positions 1, 3, 5, 7, 9, 10, 12, 14, 16 and 18 (capital letters), and 2′OMe sugar modified ribonucleotides at positions 2, 4, 6, 8, 11, 13, 15, 17 and 19 (lower case letters); and a sense strand (SEN, passenger strand) comprising unmodified ribonucleotides, an L-deoxycytidine at position 18 (lowercase bold underlined) and an inverted deoxyabasic moiety (iB) at the 5′ terminus, as depicted hereinbelow:

(antisense strand, SEQ ID NO: 11)
5′ AGGAGUUCCACAUUCUGGC 3′
(sense strand, SEQ ID NO: 12)
3′ UcCUCAAGGUGUAAGACCG-iB 5′

The RTP801 dsRNA compound (designated as “REDD14”, “DDIT4 siRNA”, PF-655″ or “PF-04523655”) used in the preparation of the composition utilized in this study is a 19-mer blunt-ended duplex having two separate strands, with an antisense strand (AS, guide strand) comprising 2′OMe sugar modified ribonucleotides at positions 1, 3, 5, 7, 9, 11, 13, 15, 17 and 19 (lower case letters), unmodified modified ribonucleotides at positions 2, 4, 6, 8, 10, 12, 14, 16 and 18 (capital letters), and a sense strand (SEN, passenger strand) comprising unmodified ribonucleotides at positions 1, 3, 5, 7, 9, 11, 13, 15, 17 and 19 (capital letters) and 2′OMe sugar modified ribonucleotides at positions 2, 4, 6, 8, 10, 12, 14, 16 and 18 (lower case letters), set forth in SEQ ID NO:9 and 10.

All dsRNA compounds were administered at a concentration of 2 μg/eye.

Retinal immunohistochemistry: Three days after lesion YFP mice were perfused with PBS and 4% paraformaldehyde (PFA) and eyes were immediately dissected. Fixed retinas were permeabilized for 3 days at 4° C. in 2% Triton X-100 and 0.5% DMSO in PBS and blocked for 1 hour in a solution of 10% Normal Goat Serum (NGS), 2% Triton X-100 (Sigma) and 0.5% DMSO in PBS. Retinas were then incubated in primary antibody overnight at 4° C. in 2% NGS, 2% Triton X-100 and 0.5% DMSO in PBS, washed, incubated in secondary antibodies for 1 hour at room temperature and washed again in PBS. The retinas were flat-mounted on glass slides with SlowFade (Invitrogen) with retina ganglion cell layer facing upwards for visualization. The primaries antibodies used were anti-SMI-32 label (1:100, Sternburger Monoclonals, Md., USA), a neurofilament-H non-phosphorylated monoclonal antibody (Lin et al. 2004) and anti-GFP (1:500, Sigma), a polyclonal antibody, to increase the signal of YFP expression. The secondary antibodies used were anti-mouse Alexa 594 (1:1000, Sigma) or anti-rabbit FITC (1:1000, Sigma).

Dendritic arbors analysis: YFP wholemount retinas were subject to neurofilament-H immunohistochemistry prior to cells imaging. Previous analyses had shown that neurofilament-H positive RGC counted for 16% of retinal RGC (data not shown). The RGCs studied meet all the following pre-requisites: i) RGCs loci were located in central half of the flatmount retina; ii) RGCs were YFP-positive with obvious axons; iii) RGCs were neurofilament-H positive; iv) and had completely visible dendritic arbors. High-resolution three-dimensional images were obtained for each cell using a Leica SP1 confocal microscope (x and y=1024×10²⁴ pixels, with 3 to 10 images averaged at each focal plane). Scans were taken at 0.48 to 0.65 μm intervals along the z axis (Objective 20× or 40×). The cells imaged were 3D-reconstructed and pre-analyzed using Imaris software (Bitplane). The following parameters were evaluated: i) Total dendritic length: the sum of the lengths of all dendrites per cell. ii) Dendritic field area: a line was drawn connecting the outermost tips of the dendrites around the edge of the arbor with dendritic field area define as the area within this contour. iii) Total number of branches: all the branches of all dendrites per cell. iv) Number of terminal branches.

Statistical analysis: Statistical analyses were performed using the GraphPad Instat software by a one-way analysis of variance (ANOVA) test, followed by nonparametric test (Dunnett's Multiple Comparison Test or Bonferroni's Test) or by Student's t test, as indicated in legends.

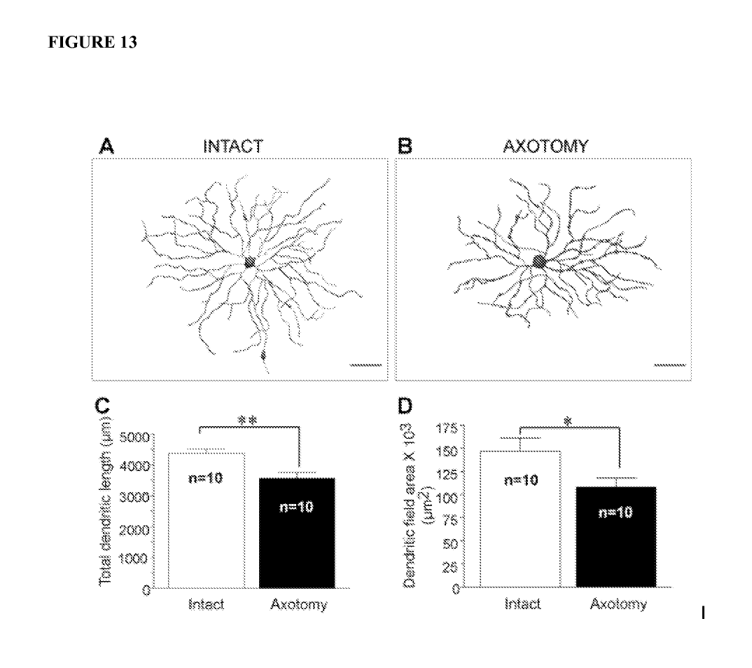

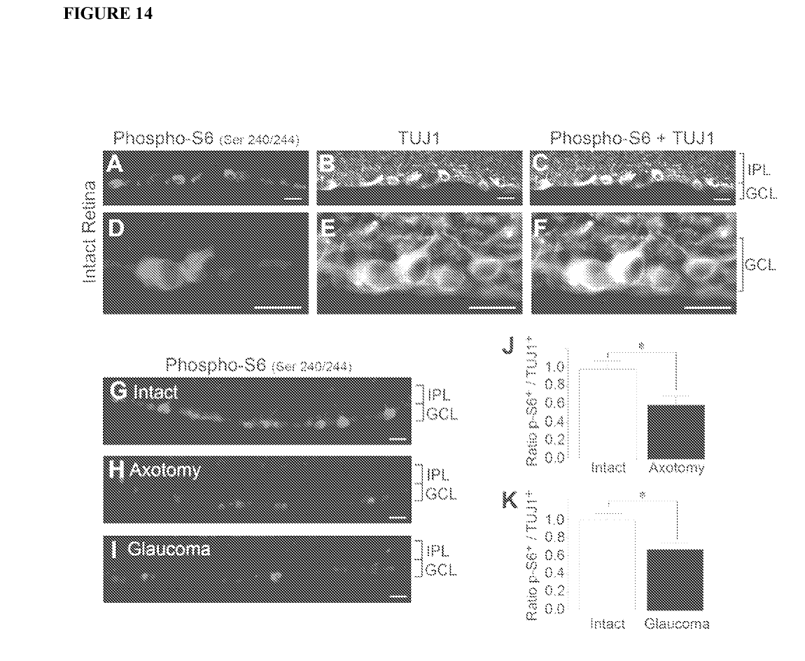

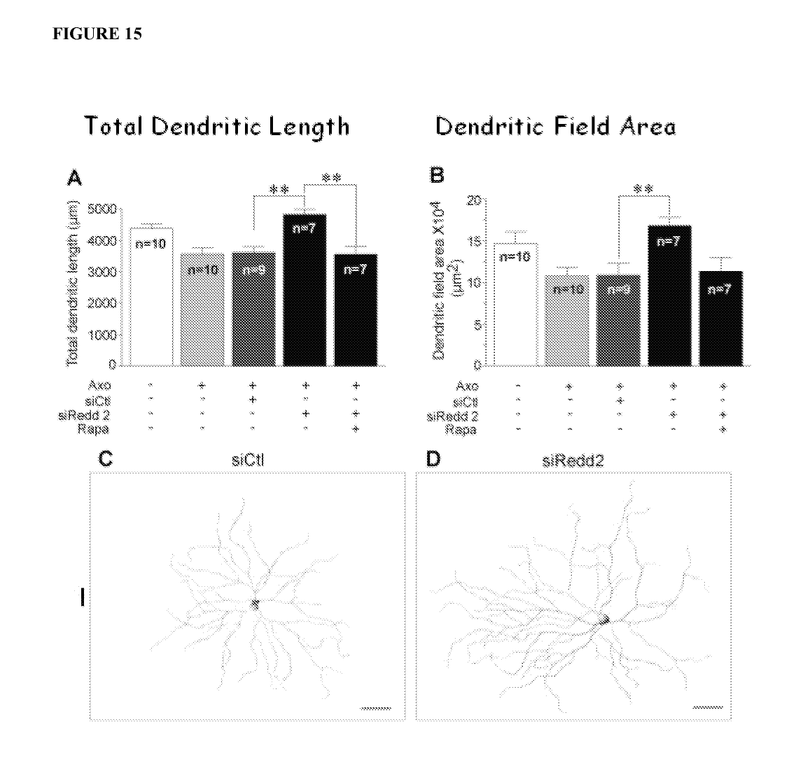

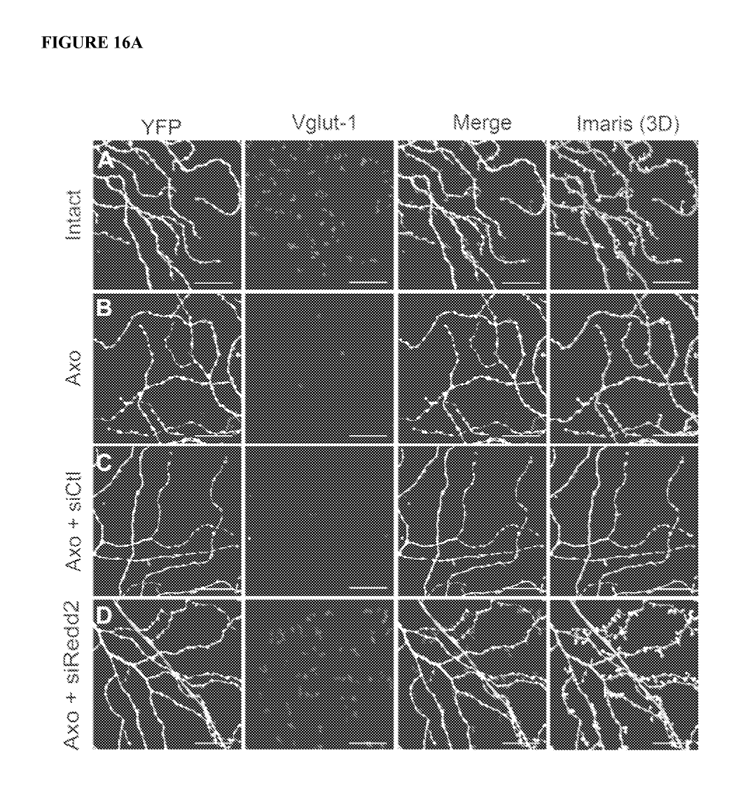

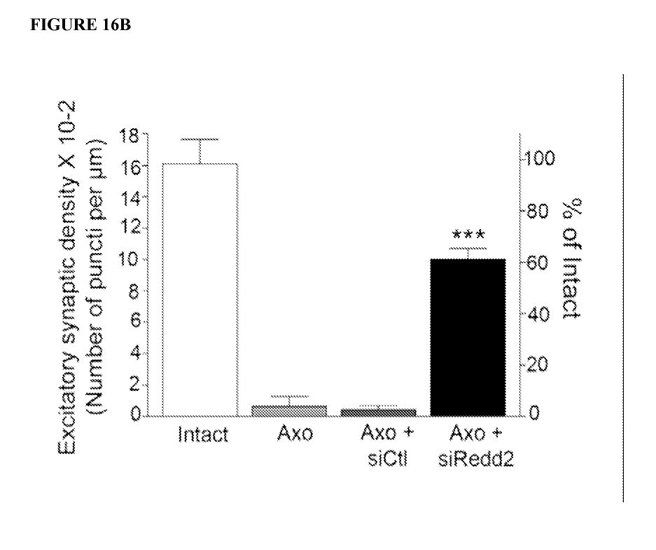

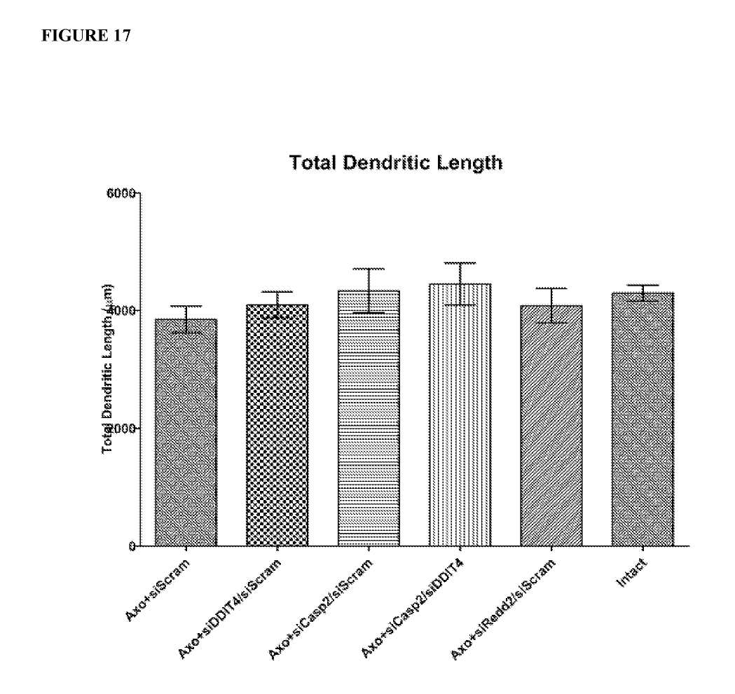

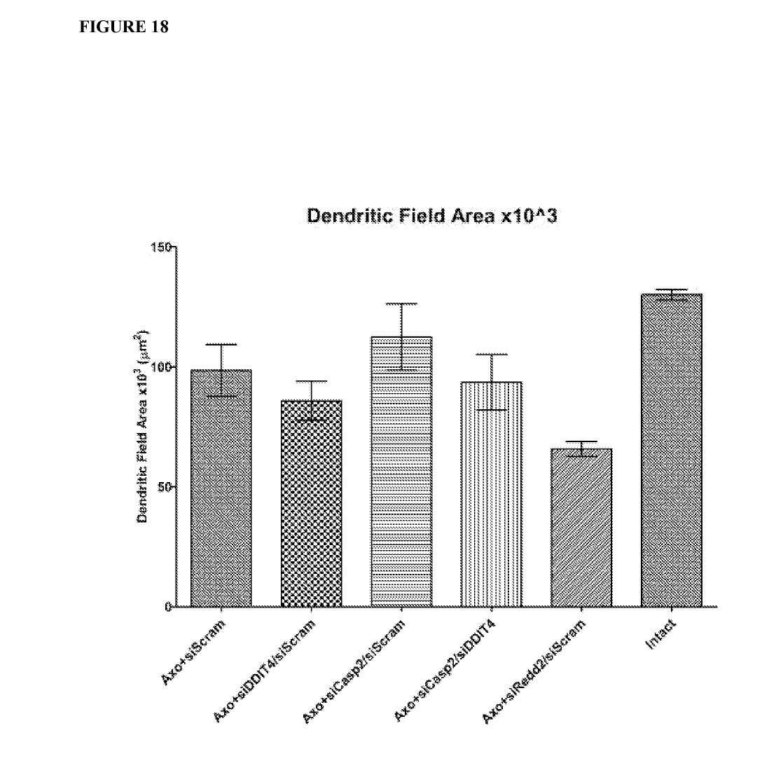

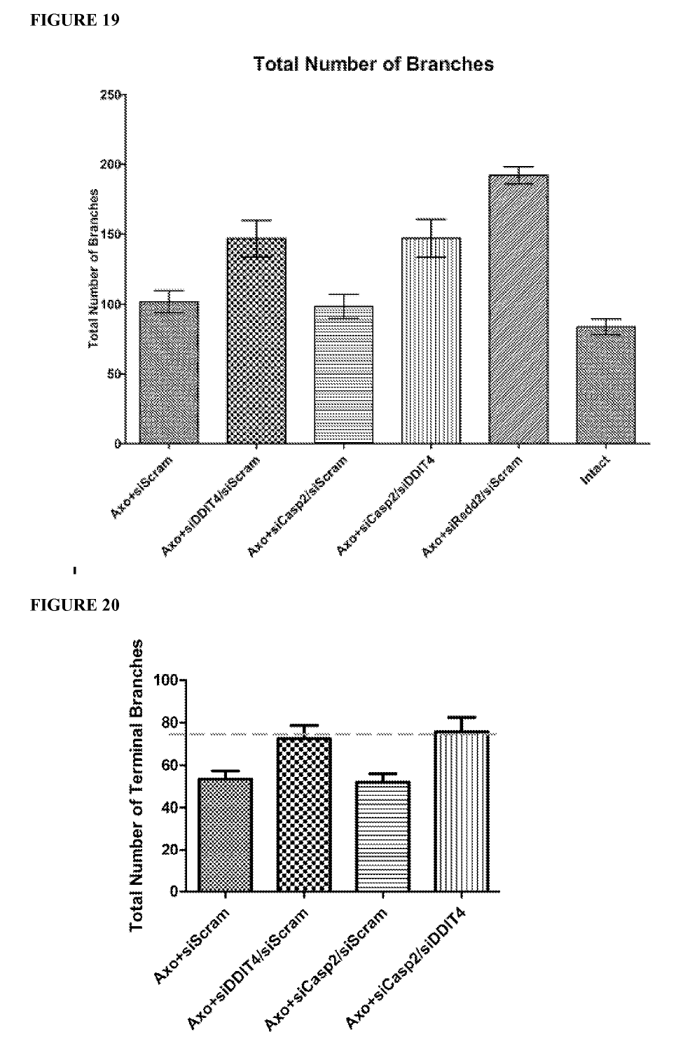

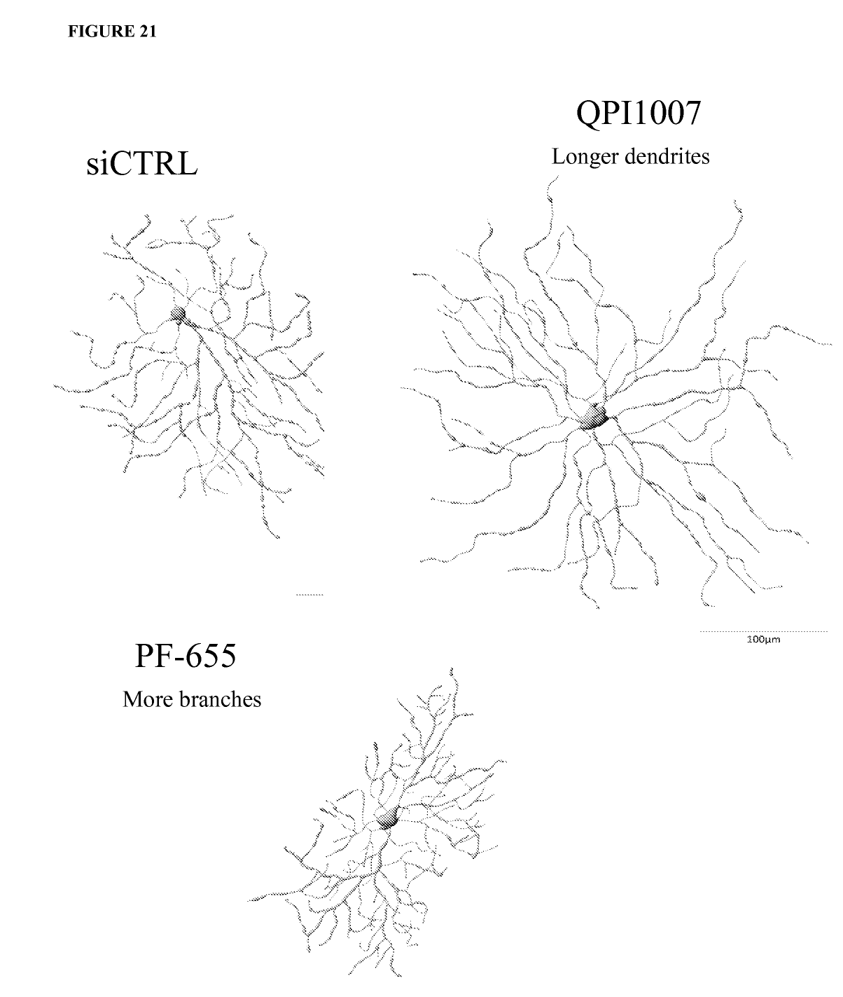

Results: Results of experiments using the RGC-YFP Transgenic Mice Model are provided in FIGS. 13-21 and in the Brief Description of the Figures section hereinabove.

The following features were studied in these experiments:
Dendritic length (influences on dendritic area);
Dendritic tree complexity, which is assessed by counting branches.

The results of these experiments are summarized in the following Table D, hereinbelow:

| siCasp2 | siRTP801 | siREDD2 | siCasp2 + siRTP801 |
|---|---|---|---|
| | | RGC dendrites | |
| | | protection | |
| | | Increases in the | |
| | | complexity of dendritic | |
| | | arbors in injured RGC | |
| | | prevents loss of | |
| | | excitatory inputs onto | |
| | | RGCs | |
| total dendritic length is | total dendritic length is | total dendritic length is | a further slight increase |
| further increased | slightly increased | slightly increased | in total dendritic length |
| (compared to | | | (compared to siCasp2 |
| siRTP801 and | | | MT) |
| siREDD2 | | | |
| monotherapies) | | | |
| increased dendritic | reduces dendritic field | more prominent | leaves the area |
| field area | area | reduction of dendritic | unchanged compared |
| | | field area (compared to | to control dsRNA |
| | | siRTP801 MT) | |
| no effect on the total | increase of the total | greater increase of the | increase in the total |
| number of dendritic | number of dendritic | total number of | number of dendritic |
| branches per neuron | branches per neuron | dendritic branches per | branches per neuron |
| | | neuron (compared to | similar to the effect |
| | | siRTP801 MT) | obtained with |
| | | | siRTP801 MT |
| no effect on the total | increase of the total | | a further slight increase |
| number of terminal | number of terminal | | in the total number of |
| branches per neuron | branches per neuron | | terminal branches per |
| compared to control | | | neuron (compared to |
| dsRNA | | | siRTP801 MT) |
| lengthened dendrites | branching | | |

*MT = monotherapy

Further results (data not shown) show that when examining the effect on the number of branches per branch order (brunch order 1-18 examined), starting from branch order 5, treatment with RTP801 inhibitor (“Axo+siDDIT4/siScram”), or with REDD2 Inhibitor (“Axo+siREDD2/siScram”), or combined treatment with RTP801 inhibitor and Casp2 inhibitor (“Axo+siDDIT4/siDDIT4”) increased the number of branches from about 15 for intact group and about 17 for control group (“Axo+siScram”), to about 21, 31 and 23, respectively for the treatment groups. The increase becomes more prominent for branches order 6 and 7, and is maintained following a gradual decrease pattern for RTP801 inhibitor, REDD2 inhibitor and combined RTP801 inhibitor and Casp2 inhibitor, for branches order 8 to 14 (for branches order 15-18 only the effect of RTP801 inhibitor remains visible). The greatest effect for all branch orders, beginning with branch orders 4 to 13, is achieved with REDD2 monotherapy.

### Example 10

### Oxygen-Induced Retinopathy (OIR) Rat Model for Evaluation of Protection of Retinal Ganglion Cells following Ischemia-Reperfusion Injury

Oxygen-Induced Retinopathy (OIR) model is a relevant model for angle closure glaucoma.

### Methods

Animal Model: Male Brown Norway rats (Japan SLC, Inc., Shizuoka) weighing approximately 200 to 250 g each were used in accordance with the ARVO Statement for the Use of Animals in Vision and Ophthalmic Research. Only one eye of each rat was used. The rats were anesthetized for all procedures with a mixture (1:1) of xylazine hydrochloride (4 mg/kg) (Bayer, Tokyo, Japan) and ketamine hydrochloride (10 mg/kg) (Sankyo, Tokyo, Japan), and the ocular surface was then anesthetized with topical instillation of 0.4% oxybuprocaine hydrochloride (Santen, Osaka, Japan). The pupils were dilated with 0.5% tropicamide and 2.5% phenylephrine hydrochloride (Santen).

Six rats were used for each group, but 12 rats were dead during procedure, and 12 eyes developed cataract, and could not obtain OCT retinal thickness data.

Ischemia-Reperfusion: The rats were placed under deep anesthesia with intramuscular injection of ketamine and xylazine. Ischemia was applied to the eye by increasing the intraocular pressure to cut off the blood supply from the retinal artery. Increased pressure was achieved by introduction of sterile saline through a 30-gauge needle that was inserted into the anterior chamber of the eye through the cornea. Each anterior chamber was cannulated with a 30-gauge infusion needle connected to a normal saline (0.9% sodium chloride) container through tubing (TI-U450P07, Terumo, Tokyo). The IOP in the cannulated eyes was raised to 90 mmHg for a period of 90 min by elevating the saline container. Intraocular pressures were measured using a rebound microtonometer designed for use on rodent eyes (TonoLab, Icare, Helsinki, Finland). Total eye ischemia was evident from the whitening of the anterior segment of the eye and the blanching of the retinal arteries on fundus examination. At the end of the ischemic period, the needle was removed from the anterior chamber, and reperfusion of the retinal vasculature was confirmed.

FG labeling: Rats were anaesthetized and sterile eye lubricant ointment was applied to prevent drying of the corneas during surgery. Head fur was shaved (from eye to ear level) and the head was fixed on the head stage by a head clamp. Operation area was disinfected with 10% povidone iodine solution followed by 70% alcohol. The point of Fluor-Gold injection was designated at a depth of 3.5 mm from the brain surface, 6.5 mm behind the bregma, 2.0 mm lateral to the midline. A hole was drilled in the skull and at the superior colliculi were injected with 2.5 micro litter of 4% FG (Fluorochrome, Inc. 529400, Englewood, Colo.)

RGC count: Labeled RGCs were counted in photographs taken from 12 areas (0.2×0.2 mm) of each retina situated, three in every retinal quadrant from the optic disc. The number of labeled cells in the photographs was divided by the area of the region to obtain mean densities of labeled cells per square millimeter, and the densities obtained in the 12 areas were pooled to calculate a mean RGC density per retina. Distinguishable glial cells (bright and small cells) were not counted. Cell counts were performed in a masked fashion.

Optical Coherence Tomography (OCT) retinal thickness analysis: OCT is based on low-coherence interferometry and provides high-resolution cross-sectional images of the retina. Retinal thickness was measured with OCT (Cirrus OCT; Carl Zeiss Meditec, Inc., Dublin, Calif.). After maximal pupillary dilatation, anesthetized rats were mounted in a head holder. The optic disc was placed in the center of the OCT image, and the scanning line was aligned to pass through both the inner and outer canthi. The scan length was 5.0 mm in all cases. Retinal thickness was measured by OCT at 1-disc diameters from the optic disc margin in the peripheral retina with an accessory program of the OCT instrument. The mean retinal thickness of one eye was defined as the average of the three measurements. All image analyses were performed in a masked fashion.

Materials: PBS, dsRNA targeting EGFP (“siGFP”), sRNA targeting REDD1 (“siRTP801”), dsRNA targeting REDD2 (“siREDD2”), dsRNA targeting Casp2 (“siCasp2”). The names, sequences and structures of the dsRNA compounds are provided in Table C.

Treatment Groups: the following treatment Groups were used:

### Intact

OIR (negative control)
OIR+intravitreal (IVT) injection of PBS (negative control)
OIR+intravitreal (IVT) injection of “siGFP” (negative control)
OIR+intravitreal (IVT) injection of “siRTP801” (REDD1)
OIR+intravitreal (IVT) injection of “siREDD2”
OIR+intravitreal (IVT) injection of “siCasp2”
OIR+intravitreal (IVT) injection of “siCasp2”+intravitreal (IVT) injection of “siRTP801”
OIR+intravitreal (IVT) injection of “siCasp2”+intravitreal (IVT) injection of “siREDD2”

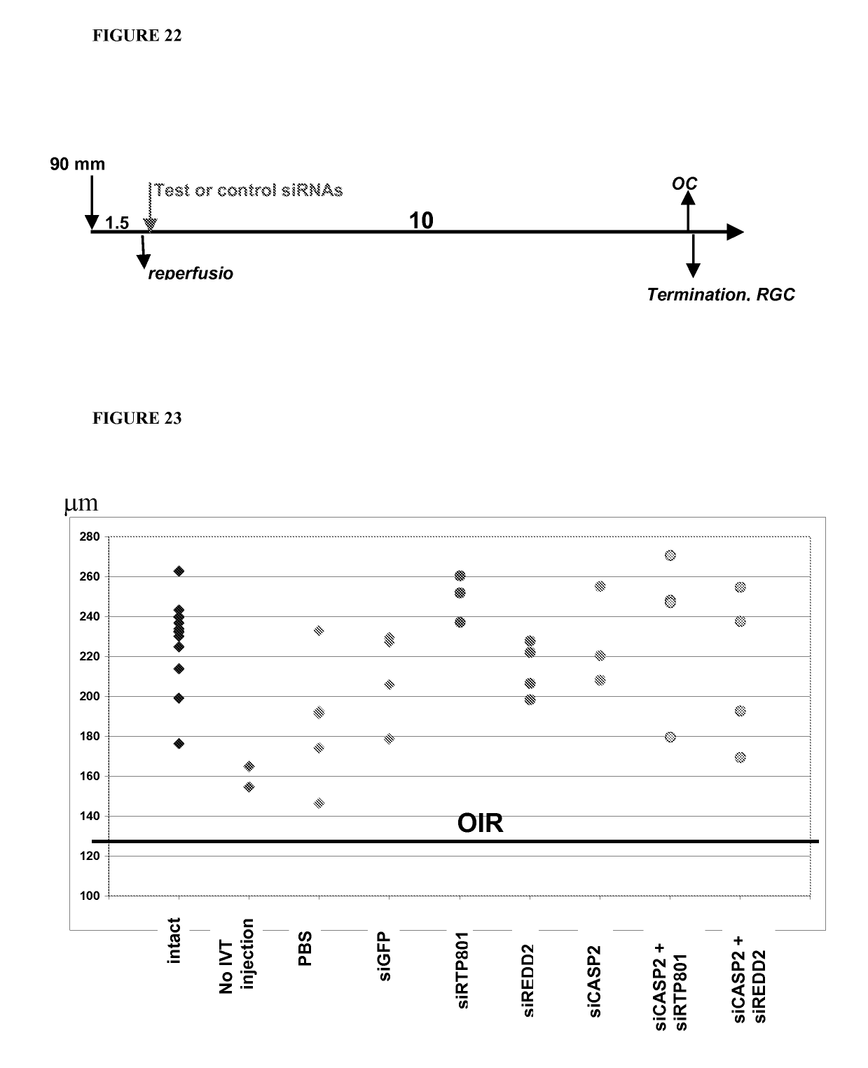

The experimental uutline is depicted in FIG. 22.

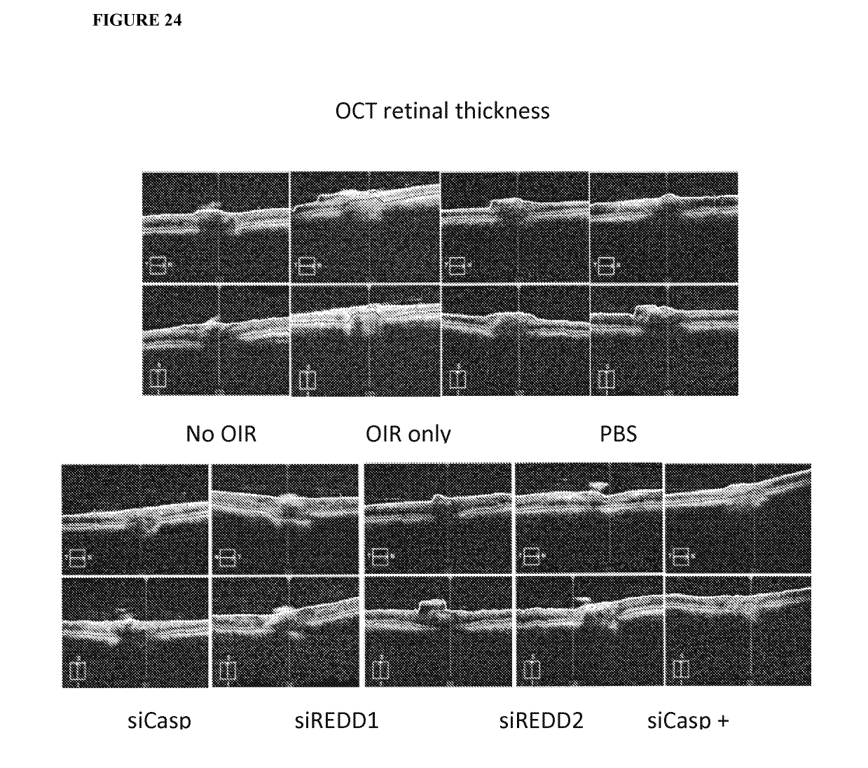

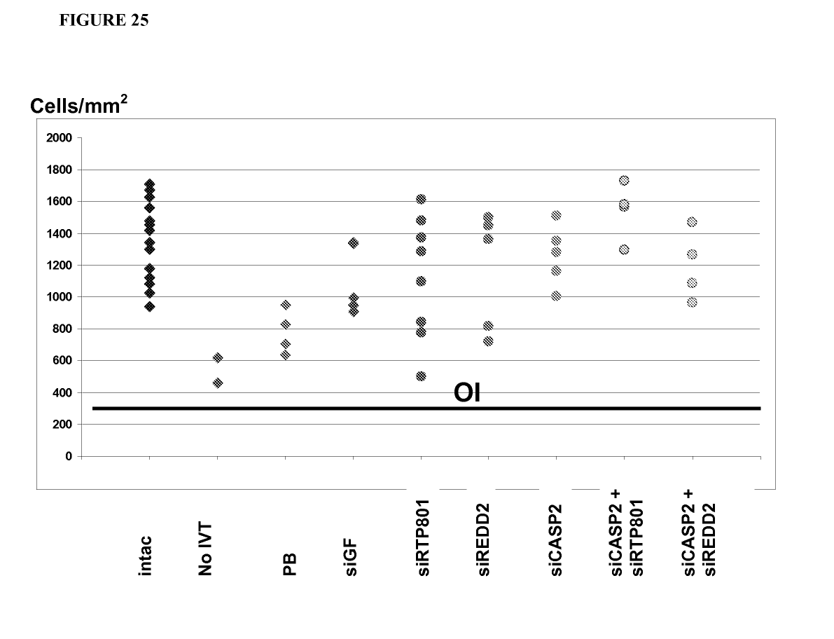

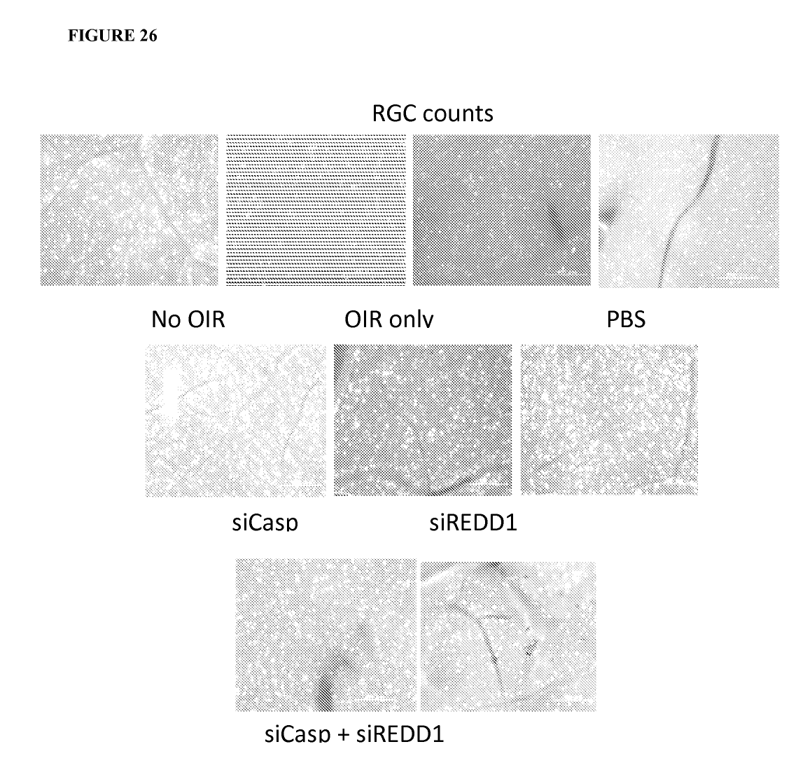

Results: Results of experiments using the Oxygen-Induced Retinopathy (OIR) Model System are provided in FIGS. 23-26 and in the Brief Description of the Figures section hereinabove.

### Conclusions

Treatment with “siCasp2” best preserved RGC. This effect was further improved by combination with siRTP801.
Treatment with “siRTP801” best preserved retinal thickness (mainly depends on the cellularity of the thickest outer cellular layer). Combination with “siCasp2” had a slightly better effect on preservation of retinal thickness.

### Example 11

### Rat Glaucoma Model for Evaluation of dsRNA Targeting RTP801 Neuroprotective Activity

The neuroprotective activity of dsRNA targeting RTP801 (“PF-655”) was evaluated in a rat glaucoma model (rat ocular hypertension model).

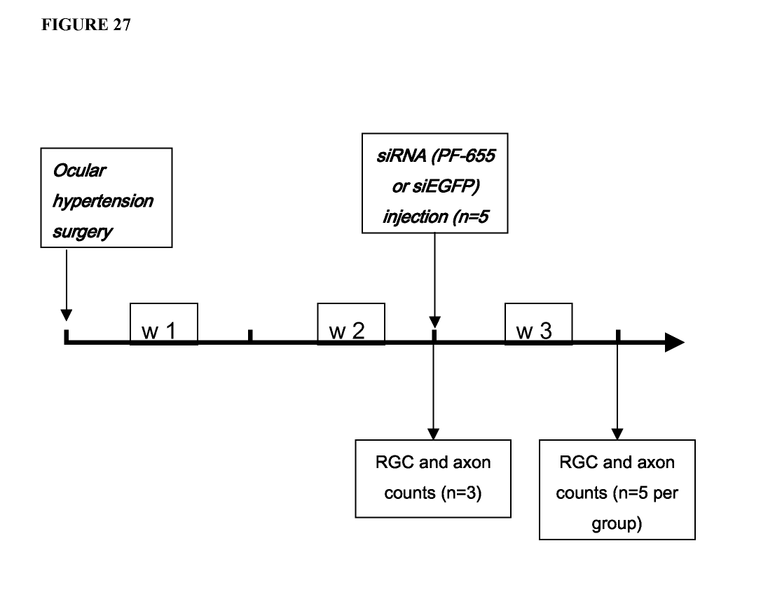

The experimental design that was performed using this model system is depicted in FIG. 27.

For evaluation, rats were deeply anesthetized and perfused transcardially with 4% paraformaldehyde (PFA) in 0.1 M phosphate buffer. Both eyes were immediately enucleated and retinas were dissected and flat-mounted on a glass slide with the ganglion cell layer side up for quantification of RGC soma. Optic nerves were dissected and fixed in 2% PFA and 2.5% glutaraldehyde in 0.1 M phosphate buffer for 24 hours, followed by fixation in 2% osmium tetroxide, and embedding in epon resin. Semi-thin cross-sections (0.7-μm-thick) were cut on a microtome (Reichert, Vienna, Austria) and stained with 1% toluidine blue. RGC axons were counted at 1 mm from the optic nerve head in five non-overlapping areas of each optic nerve section, encompassing a total area of 5500 μm2 per nerve. The five optic nerve areas analyzed included: one in the center of the nerve, two peripheral dorsal and two peripheral ventral regions. The total area per optic nerve cross-section was measured using Northern Eclipse image analysis software (Empix Imaging, Toronto, ON, Canada), and this value was used to estimate the total number of axons per optic nerve.

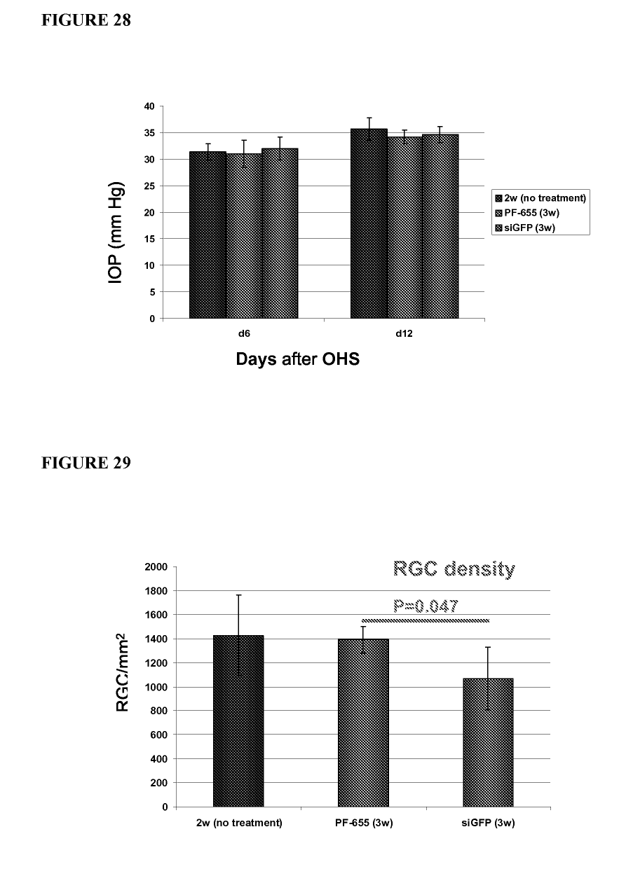

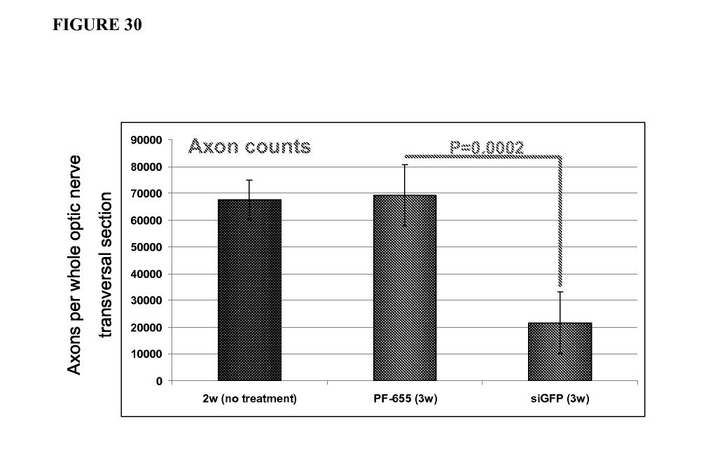

Results: Results of experiments using the Glaucoma Rat Model System for Evaluation of dsRNA targeting RTP801 Neuroprotective Activity are provided in FIGS. 28-30 and in the Brief Description of the Figures section hereinabove.

Results obtained in this experimental model can be summarized as follows:

Intravitreal treatment with “PF-655” at 2 weeks after establishment of ocular hypertension resulted in:
No further RGC body loss compared to pre-treatment baseline;
No further RGC axon loss compared to pretreatment baseline.

The protective effects of “PF-655” towards both RGC bodies and axons were statistically significant compared to the group treated with control “siGFP”.

The nerves from eyes treated with “siGFP” looked considerably more damaged, often reflecting more axonal loss than cell body loss, which is characteristic of glaucomatous damage.

The protection of RGC axons was numerically more impressive than that of RGC bodies: 1.3-fold more RGC bodies and 3.2-fold more RGC axons survived till the end of w3 of ocular hypertension in “PF-655”-treated eyes compared to “siGFP”-treated eyes.

Overall, the results convincingly show that “PF-655” promotes robust protection of RGC bodies and axons in rat ocular hypertension model.

### Example 12

### Rat Axotomy Model for Evaluation of the Neuroprotective Effect of dsRNA targeting Casp2, RTP801 and their Combination after Administration by Intravitreal (IVT) Injection

Evaluation of the neuroprotective effect of Casp2 dsRNA, RTP801 dsRNA and their combination after administration by intravitreal (IVT) is performed in Rat Axotomy Model at two (2) weeks post injury.

Objective: to evaluate potential additive or synergistic effects of dsRNAs targeting Casp2 or RTP801 in RGC neuroprotection in optic nerve axotomy model.

Methodology: Optic nerve axotomy was performed in adult rats. Immediately after surgery, rats received intravitreal injections (injection volume was 5 uL) with dsRNA targeting EGFP (negative control) or with the following combinations of dsRNAs: Casp2+RTP801; Casp2+EGFP; RTP801+EGFP. Second similar injections were performed into corresponding eyes at 1 week after axotomy. The dsRNA compounds were administered at 20 ug per dsRNA compound in combination or 40 ug for “siEGFP” alone) Evaluation of the neuroprotective effects of each of the treatments was performed by counting of FG relabeled RGC in retinal whole mounts at 2 weeks after axotomy.

Total number of animals: 25

TABLE E

| | | | | | | Termination |
|---|---|---|---|---|---|---|
| | | | | Administration | | (weeks post |
| Group | N | Axotomy | Treatment | Route | Treatment Regime | axotomy) |
| 1 | 5 | Yes | siCasp2 + siEGFP | Unilateral IVT | Day 0 and Day 7 | 2 |
| 2 | 5 | Yes | siRTP801 + siEGFP | Unilateral IVT | Day 0 and Day 7 | 2 |
| 3 | 5 | Yes | siRTP801 + siCasp2 | Unilateral IVT | Day 0 and Day 7 | 2 |
| 4 | 5 | Yes | EGFP | Unilateral IVT | Day 0 and Day 7 | 2 |
| 5 | 5 | — | Intact | — | — | — |

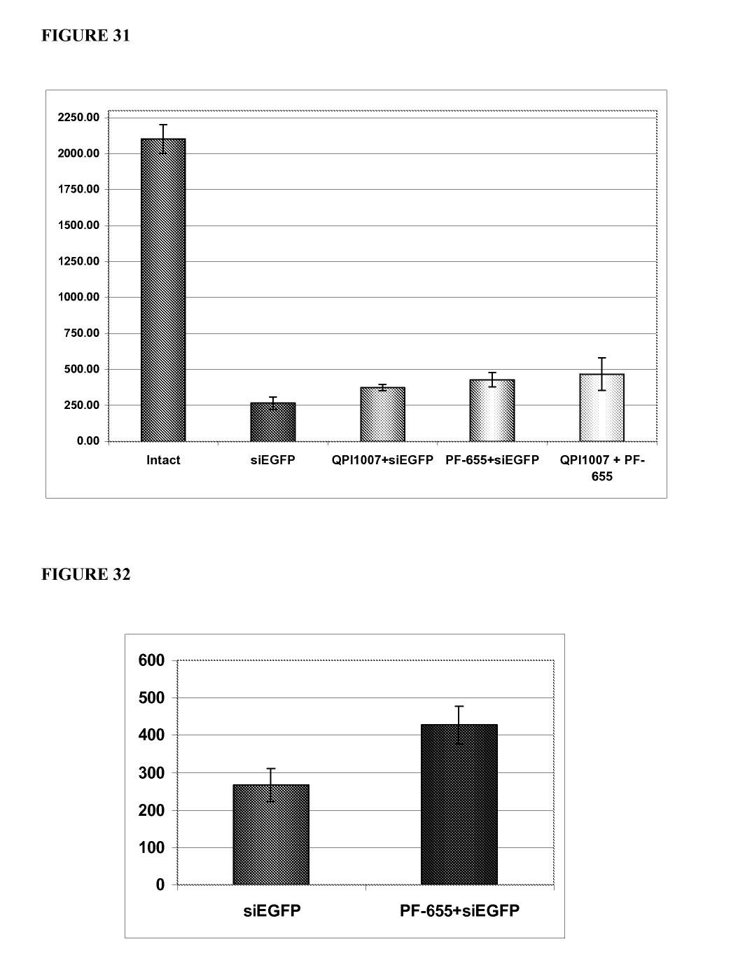

Results: Results of experiments using the Rat Axotomy Model for Evaluation neuroprotective effect of Casp2 dsRNA, RTP801 dsRNA and their combination after administration by intravitreal (IVT), are provided in FIGS. 31-33 and in the Brief Description of the Figures section hereinabove. Evaluation of the neuroprotective effects of each of the treatments was performed by counting of FG relabeled RGC in retinal whole mounts at 2 weeks after axotomy.

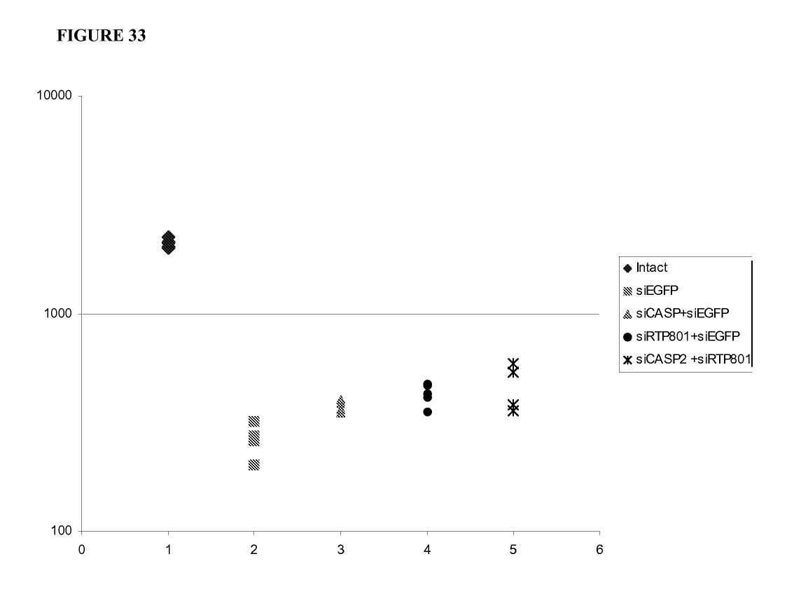

Treatment with “QPI1007”+“siEGFP” or “PF-655”+“siEGFP” significantly increased RGC survival compared to siEGFP (FIGS. 31 and 33). FIG. 32 also shows the level of neuroprotection (RGC survival) achieved in “PF-655”+“siEGFP” treated eyes. The neuroprotective effects of RTP801 inhibitor was enhanced by the combinatorial administration of Casp2 inhibitor (“QPI1007”) and RTP801 inhibitor (“PF-655’). FIGS. 31 and 33 show that when given together, “PF-655” and “QPI1007” increased RGC survival compared to “QPI1007” or “PF-655” monotherapy.

The methods and compositions disclosed herein have been described broadly and generically. Each of the narrower species and subgeneric groupings falling within the generic disclosure also form part of the disclosure. This includes the generic description of the disclosure with a proviso or negative limitation removing any subject matter from the genus, regardless of whether or not the removed material is specifically recited herein. Other embodiments are within the following claims.

1. A therapeutic combination comprising a RTP801 inhibitor or a REDD2 inhibitor; and a Casp2 inhibitor for use in treating a subject suffering from, or at risk of, developing a disease.

2. The therapeutic combination of claim 1, wherein each of the RTP801 inhibitor or the REDD2 inhibitor; and the Casp2 inhibitor is independently selected from the group consisting of an antibody, a polypeptide, a peptide, a nucleic acid molecule and a small organic molecule.

3. The therapeutic combination of claim 1 or 2, wherein each of the RTP801 inhibitor or the REDD2 inhibitor; and the Casp2 inhibitor is independently a nucleic acid molecule.

4. The therapeutic combination of claim 3, wherein the nucleic acid molecule comprises a double-stranded RNA (dsRNA) compound comprising an antisense strand and a sense strand.

5. The therapeutic combination of claim 4, wherein the RTP801 inhibitor comprises a RTP801 double-stranded RNA compound, wherein the antisense strand comprises the sequence:
(SEQ ID NO: 7)
5′ AGCUGCAUCAGGUUGGCAC 3′.

6. The therapeutic combination of claim 4, wherein the REDD2 inhibitor comprises a REDD2 double-stranded RNA compound.

7. The therapeutic combination of any one of claims 4-6, wherein the antisense strand of the Casp2 double-stranded RNA compound comprises the sequence:

| | |
|---|---|
| 5′ AGGAGUUCCACAUUCUGGC 3′. | (SEQ ID NO: 11) |

8. The therapeutic combination of claim 5, wherein the RTP801 double-stranded RNA compound has the structure:
(antisense strand, SEQ ID NO: 9)
5′ AGCUGCAUCAGGUUGGCAC 3′
(sense strand; SEQ ID NO: 10)
3′ UCGACGUAGUCCAACCGUG 5′
wherein the ribonucleotide at the 5′ terminus and the ribonucleotide at the 3′ terminus of the antisense strand is a 2′-O-methyl sugar modified ribonucleotide;
wherein the ribonucleotide at the 5′ terminus and the ribonucleotide at the 3′ terminus of the sense strand is an unmodified ribonucleotide;
wherein the remaining ribonucleotides in the antisense strand and in the sense strand comprise alternating unmodified ribonucleotides and 2′-O-methyl sugar modified ribonucleotides;
wherein the ribonucleotide at each of the 5′ terminus and the 3′ terminus of the antisense strand and the sense strand is independently phosphorylated or non-phosphorylated; and
wherein the Casp2 double-stranded RNA compound has the structure:
(antisense strand, SEQ ID NO: 13)
5′ AGGAGUUCCACAUUCUGGC 3′
(sense strand, SEQ ID NO: 14)
3′ UCCUCAAGGUGUAAGACCG-iB 5′
wherein each A, C, U and G is joined to the next A, C, U, and G by a covalent bond;
wherein the sense strand comprises, counting from the 5′ terminus, an unmodified ribonucleotide at positions 1, 2, 3, 4, 5, 6, 7, 8, 9, 10, 11, 12, 13, 14, 15, 16, 17 and 19, a L-deoxycytidine at position 18, and an inverted abasic 5′ cap; and
wherein the antisense strand comprises, counting from the 5′ terminus, 2′-O-methyl sugar modified ribonucleotide at positions 2, 4, 6, 8, 11, 13, 15, 17 and 19 and unmodified ribonucleotide at positions 1, 3, 5, 7, 9, 10, 12, 14, 16 and 18.

9. The therapeutic combination of claim 8, wherein the covalent bond joining each A, C, U and G to the next A, C, U and G is a phosphodiester bond.

10. The therapeutic combination of any one of claims 1-9, wherein the disease comprises neurodegeneration.

11. The therapeutic combination of claim 10, wherein the disease is associated with a physically damaged nerve and/or neurite damage.

12. The therapeutic combination of any one of claims 1-11, wherein the disease is selected from the group consisting of an ocular disease, an ocular disorder and an ocular injury.

13. The therapeutic combination of claim 12, wherein the ocular injury is selected from the group consisting of ischemic injury, ischemia-reperfusion injury, mechanical injury, and injury or interruption of nerve fibers, and/or is associated with lack of retrograde supply of neurotrophic factor.

14. The therapeutic combination of any one of claims 1-13, wherein the disease is selected from the group consisting of physical damage to the central and/or peripheral nervous system; brain damage associated with stroke, amyotrophic lateral sclerosis (ALS), multiple sclerosis (MS), progressive muscular atrophy, Gullain-Barre syndrome, Alzheimer's disease, Huntington's Disease, Parkinson's disease, episodic vertigo, hearing loss, tinnitus and aural fullness, diabetic neuropathy, increased intraocular pressure, open angle glaucoma, angle closure glaucoma, diabetic retinopathy (DR), diabetic macular edema (DME), age related macular degeneration (AMD), Leber's hereditary optic neuropathy (LHON), Leber's optic atrophy, optic neuritis, retinal artery occlusion, central retinal vein occlusion, branch retinal vein occlusion, ischemic optic neuropathy including non-arteritic ischemic optic neuropathy (NAION), optic nerve injury, retinopathy of prematurity (ROP) or retinitis pigmentosa (RP), retinal ganglion degeneration, macular degeneration, hereditary optic neuropathy, metabolic optic neuropathy, optic neuropathy due to a toxic agent and neuropathy caused by adverse drug reactions or a vitamin deficiency.

15. The therapeutic combination of any one of claims 1-14, wherein the RTP801 inhibitor or the REDD2 inhibitor is configured for simultaneous administration with the Casp2 inhibitor.

16. The therapeutic combination of any one of claims 1-14, wherein the RTP801 inhibitor or the REDD2 inhibitor is configured for administration prior to administration of the Casp2 inhibitor.

17. The therapeutic combination of any one of claims 1-14, wherein the RTP801 inhibitor or the REDD2 inhibitor is configured for administration subsequent to administration of the Casp2 inhibitor.

18. The therapeutic combination of any one of claims 1-17, wherein the RTP801 inhibitor or the REDD2 inhibitor; and the Casp2 inhibitor are configured for administration in different doses.

19. The therapeutic combination of claim 15, wherein the RTP801 inhibitor is a double-stranded RNA compound, wherein the Casp2 inhibitor is a double-stranded RNA compound; and wherein the RTP801 inhibitor and the Casp2 inhibitor are configured for administration in a ratio from 1:1 to 1000:1 RTP801 inhibitor:Casp2 inhibitor.

20. A therapeutic combination comprising a RTP801 inhibitor or a REDD2 inhibitor; and a Casp2 inhibitor for use in providing neuroprotection to a neuron in a subject in need thereof.

21. The therapeutic combination of claim 20, wherein the neuron is, or is comprised within, a system selected from the group consisting of a peripheral nervous system, a central nervous system and an audio-vestibular system.

22. The therapeutic combination of claim 21, wherein the neuron is, or is comprised within, a visual system of a central nervous system.

23. The therapeutic combination of claim 20, wherein the neuron is a ganglion cell.

24. The therapeutic combination of claim 23, wherein the ganglion cell is selected from the group consisting of a retinal ganglion cell, a spiral ganglion cell, a vestibular ganglion cell, a dorsal ganglion cell and a peripheral ganglion cell.

25. The therapeutic combination of any one of claims 20-24, wherein the neuroprotection comprises protecting the neuron from death.

26. The therapeutic combination of claim 25, wherein death of the neuron is associated with one or more of a disease or disorder, a surgery, ischemia, ischemia/reperfusion, physical/mechanical trauma, a chemical agent, an infectious agent, an immunologic reaction and a nutritional imbalance.

27. A composition comprising an RTP801 inhibitor or a REDD2 inhibitor; and a Casp2 inhibitor, and a pharmaceutically acceptable carrier.

28. The composition of claim 27, wherein the RTP801 inhibitor or the REDD2 inhibitor is a double-stranded RNA compound comprising a sense strand and an antisense strand.

29. The composition of claim 28, comprising an RTP801 double-stranded RNA compound having an antisense strand comprising the sequence.

| | |
|---|---|
| 5′ AGCUGCAUCAGGUUGGCAC 3′. | (SEQ ID NO: 7) |

30. The composition of any one of claims 27-29, wherein the Casp2 inhibitor is a double-stranded RNA compound comprising a sense strand and an antisense strand.

31. The composition of claim 30, wherein the antisense strand of the Casp2 double-stranded RNA compound comprises the sequence:

| | |
|---|---|
| 5′ AGGAGUUCCACAUUCUGGC 3′. | (SEQ ID NO: 11) |

32. The composition of any one of claims 29-31, comprising an RTP801 double-stranded RNA compound and a Casp2 double-stranded RNA compound present in a ratio from 1:1 to 1000:1 RTP801 inhibitor:Casp2 inhibitor.

33. The composition of any one of claims 27-32, for use in the treatment of a subject suffering from or at risk of developing a disease, a disorder or an injury.

34. The composition of claim 33, wherein the disease, disorder, or injury is associated with a physically damaged nerve and/or neurite damage.

35. The composition of claim 33 or 34, wherein the disease is selected from the group consisting of an ocular disease, an ocular disorder and an ocular injury.

36. The composition of claim 35, wherein the ocular injury comprises ischemic injury, ischemia-reperfusion injury, mechanical injury, injury or interruption of nerve fibers and/or is associated with lack of retrograde supply of neurotrophic factor.

37. The composition of any one of claims 33-36, wherein the disease is selected from the group consisting of physical damage to the central and/or peripheral nervous system; brain damage associated with stroke, amyotrophic lateral sclerosis (ALS), multiple sclerosis (MS), progressive muscular atrophy, Gullain-Barre syndrome, Alzheimer's disease, Huntington's Disease, Parkinson's disease, episodic vertigo, hearing loss, tinnitus and aural fullness, diabetic neuropathy, increased intraocular pressure, open angle glaucoma, angle closure glaucoma, diabetic retinopathy (DR), diabetic macular edema (DME), age related macular degeneration (AMD), Leber's hereditary optic neuropathy (LHON), Leber's optic atrophy, optic neuritis, retinal artery occlusion, central retinal vein occlusion, branch retinal vein occlusion, ischemic optic neuropathy including non-arteritic ischemic optic neuropathy (NAION), optic nerve injury, retinopathy of prematurity (ROP) or retinitis pigmentosa (RP), retinal ganglion degeneration, macular degeneration, hereditary optic neuropathy, metabolic optic neuropathy, optic neuropathy due to a toxic agent and neuropathy caused by adverse drug reactions or a vitamin deficiency.

38. An inhibitor selected from the group consisting of a RTP801 inhibitor or a salt thereof and a REDD2 inhibitor or a salt thereof, for use in promoting neurite outgrowth, axonal regeneration and/or neural regeneration, wherein the inhibitor is configured for contacting a neuron.

39. An inhibitor selected from the group consisting of a RTP801 inhibitor or a salt thereof and a REDD2 inhibitor or a salt thereof, for use in promoting neurite outgrowth, axonal regeneration and/or neural regeneration in a subject in need thereof.

40. An inhibitor selected from the group consisting of a RTP801 inhibitor and a REDD2 inhibitor for use in maintaining the viability of a neuron in a peripheral nervous system and/or a central nervous system, including a visual system, and/or an audio-vestibular system, wherein the inhibitor is configured for contacting the neuron.

41. An inhibitor of expression or activity of RTP801 or of REDD2 for use in preventing, treating, or reducing symptoms of nerve injury in a subject.

42. An inhibitor selected from the group consisting of an RTP801 inhibitor and a REDD2 inhibitor for use in treating nerve damage in a subject in need thereof.

43. The inhibitor of any one of claims 38-42, wherein the RTP801 inhibitor or the REDD2 inhibitor is selected from the group consisting of an antibody, a polypeptide, a peptide, a nucleic acid molecule and a small organic molecule.

44. The inhibitor of claim 43, wherein the RTP801 inhibitor or the REDD2 inhibitor is a nucleic acid molecule.

45. The inhibitor of claim 44, wherein the nucleic acid molecule is a double-stranded RNA (dsRNA) compound.

46. The inhibitor of claim 45, wherein the double-stranded RNA compound comprises an antisense strand and a sense strand.

47. The inhibitor of claim 46, wherein the antisense strand of the RTP801 inhibitor double-stranded RNA compound comprises the sequence:

| | |
|---|---|
| 5′ AGCUGCAUCAGGUUGGCAC 3′. | (SEQ ID NO: 7) |

48. The inhibitor of claim 47, wherein the RTP801 double-stranded RNA compound has the structure:
(antisense strand, SEQ ID NO: 9)
5′ AGCUGCAUCAGGUUGGCAC 3′
(sense strand, SEQ ID NO: 10)
3′ UCGACGUAGUCCAACCGUG 5′.

49. The inhibitor of claims 38-48, wherein the neuron or nerve is, or is comprised within a system selected from a peripheral nervous system and a central nervous system, and an audio-vestibular system.

50. The inhibitor of claim 49, wherein the neuron or nerve is, or is comprised within, a visual system of a central nervous system.

51. The inhibitor of claims 38-50, wherein the neuron is a ganglion cell.

52. The inhibitor of claim 51, wherein the ganglion cell is selected from the group consisting of a retinal ganglion cell, a spiral ganglion cell, a vestibular ganglion cell, dorsal root ganglion and a peripheral ganglion cell.

53. The inhibitor of claims 38-50, wherein the neuron is derived from a stem cell or from a progenitor cell.

54. The inhibitor of claim 53, wherein the stem cell is Muller's glia.

55. An RTP801 inhibitor or a REDD2 inhibitor for use in the treatment of a disease or condition benefiting from promotion of neuronal growth and/or repair.

56. A composition comprising an RTP801 inhibitor or a REDD2 inhibitor, for use in promoting neurite outgrowth, axonal regeneration or neural regeneration.

57. A composition comprising an RTP801 inhibitor or a REDD2 inhibitor, for use in promoting neurite outgrowth, axonal regeneration or neural regeneration within the optic nerve.

58. A kit comprising an RTP801 inhibitor or a REDD2 inhibitor; and a Casp2 inhibitor;
and instructions for use.

59. The kit of claim 58, wherein the use is for treatment of a disease, disorder, or injury comprising neurodegeneration and/or associated with a physically damaged nerve and/or neurite damage.

60. The kit of claim 59, wherein the use is for treatment of an ocular disease, an ocular disorder or an ocular injury.

61. The kit of claim 60, wherein the ocular injury comprises ischemic injury, ischemia-reperfusion injury, mechanical injury, injury or interruption of nerve fibers and/or is associated with lack of supply of neurotrophic factor.

62. The kit of claim 59, wherein the disease is selected from the group consisting of physical damage to the central and/or peripheral nervous system; brain damage associated with stroke, amyotrophic lateral sclerosis (ALS), multiple sclerosis (MS), progressive muscular atrophy, Gullain-Barre syndrome, Alzheimer's disease, Huntington's Disease, Parkinson's disease, episodic vertigo, hearing loss, tinnitus and aural fullness, diabetic neuropathy, increased intraocular pressure, open angle glaucoma, angle closure glaucoma, diabetic retinopathy (DR), diabetic macular edema (DME), age related macular degeneration (AMD), Leber's hereditary optic neuropathy (LHON), Leber's optic atrophy, optic neuritis, retinal artery occlusion, central retinal vein occlusion, branch retinal vein occlusion, ischemic optic neuropathy including . . . , optic nerve injury, retinopathy of prematurity (ROP) or retinitis pigmentosa (RP), retinal ganglion degeneration, macular degeneration, hereditary optic neuropathy, metabolic optic neuropathy, optic neuropathy due to a toxic agent and neuropathy caused by adverse drug reactions or a vitamin deficiency.

63. A kit comprising an RTP801 inhibitor or a REDD2 inhibitor; and instructions for use in promoting neurite outgrowth, axonal regeneration or neural regeneration.

64. A method of treating a subject suffering from or at risk of developing a disease which comprises administering to the subject a therapeutically effective amount of a RTP801 inhibitor or a REDD2 inhibitor; and a therapeutically effective amount of a Casp2 inhibitor, so as to thereby treat the subject.

65. Use of a therapeutic combination comprising a RTP801 inhibitor or a REDD2 inhibitor; and a Casp2 inhibitor in the manufacture of a medicament for treating a subject suffering from or at risk of developing a disease.

66. The method or use of claim 64 or 65, wherein each of the RTP801 inhibitor, or the REDD2 inhibitor; and the Casp2 inhibitor is independently selected from the group consisting of an antibody, a polypeptide, a peptide, a nucleic acid molecule and a small organic molecule.

67. The method or use of claim 66, wherein each of the RTP801 inhibitor, or the REDD2 inhibitor; and the Casp2 inhibitor is independently a nucleic acid molecule.

68. The method or use of claim 67, wherein the nucleic acid molecule comprises a double-stranded RNA (dsRNA) compound comprising an antisense strand and a sense strand.

69. The method or use of claim 68, wherein the RTP801 inhibitor comprises a RTP801 double-stranded RNA compound, wherein the antisense strand comprises the sequence:
(SEQ ID NO: 7)
5′ AGCUGCAUCAGGUUGGCAC 3′.

70. The method or use of claim 68 wherein the REDD2 inhibitor comprises a REDD2 double-stranded RNA compound.

71. The method or use of any one of claims 68-70, wherein the antisense strand of the Casp2 double-stranded RNA compound comprises the sequence:
(SEQ ID NO: 11)
5′ AGGAGUUCCACAUUCUGGC 3′.

72. The method or use of claim 71, wherein the RTP801 double-stranded RNA compound has the structure:
(antisense strand, SEQ ID NO: 9)
5′ AGCUGCAUCAGGUUGGCAC 3′
(sense strand; SEQ ID NO: 10)
3′ UCGACGUAGUCCAACCGUG 5′
wherein the ribonucleotide at the 5′ terminus and the ribonucleotide at the 3′ terminus of the antisense strand is a 2′-O-methyl sugar modified ribonucleotide;
wherein the ribonucleotide at the 5′ terminus and the ribonucleotide at the 3′ terminus of the sense strand is an unmodified ribonucleotide;
wherein the remaining ribonucleotides in the antisense strand and in the sense strand comprise alternating unmodified ribonucleotides and 2′-O-methyl sugar modified ribonucleotides;
wherein the ribonucleotide at each of the 5′ terminus and the 3′ terminus of the antisense strand and the sense strand is independently phosphorylated or non-phosphorylated; and
wherein the Casp2 double-stranded RNA compound has the structure:
(antisense strand, SEQ ID NO: 13)
5′ iB-GCCAGAAUGUGGAACUCCU 3′
(sense strand; SEQ ID NO: 14)
3′ CGGUCUUACACCUUGAGGA 5′
wherein each A, C, U, and G is joined to the next A, C, U, and G by a covalent bond;
wherein the sense strand comprises, counting from the 5′ terminus, an unmodified ribonucleotide at positions 1, 2, 3, 4, 5, 6, 7, 8, 9, 10, 11, 12, 13, 14, 15, 16, 17 and 19, a L-deoxycytidine at position 18, and an inverted abasic 5′ cap; and
wherein the antisense strand comprises, counting from the 5′ terminus, 2′-O-methyl sugar modified ribonucleotide at positions 2, 4, 6, 8, 11, 13, 15, 17 and 19 and unmodified ribonucleotide at positions 1, 3, 5, 7, 9, 10, 12, 14, 16 and 18.

73. The method or use of claim 72, wherein the covalent bond joining each A, C, U, and G to the next A, C, U, and G is a phosphodiester bond.

74. The method or use of any one of claims 64-73, wherein the disease comprises neurodegeneration.

75. The method or use of claim 74, wherein the disease is associated with a physically damaged nerve and/or neurite damage.

76. The method or use of any one of claims 64-74, wherein the disease is selected from the group consisting of an ocular disease, an ocular disorder and an ocular injury.

77. The method or use of claim 76, wherein the ocular injury is selected from the group consisting of ischemic injury, ischemia-reperfusion injury, mechanical injury, and injury or interruption of nerve fibers, and/or is associated with lack of retrograde supply of neurotrophic factor.

78. The method or use of any one of claims 64-77, wherein the disease is selected from the group consisting of physical damage to the central and/or peripheral nervous system; brain damage associated with stroke, amyotrophic lateral sclerosis (ALS), multiple sclerosis (MS), progressive muscular atrophy, Gullain-Barre syndrome, Alzheimer's disease, Huntington's Disease, Parkinson's disease, episodic vertigo, hearing loss, tinnitus and aural fullness, diabetic neuropathy, increased intraocular pressure, open angle glaucoma, angle closure glaucoma, diabetic retinopathy (DR), diabetic macular edema (DME), age related macular degeneration (AMD), Leber's hereditary optic neuropathy (LHON), Leber's optic atrophy, optic neuritis, retinal artery occlusion, central retinal vein occlusion, branch retinal vein occlusion, ischemic optic neuropathy, optic nerve injury, retinopathy of prematurity (ROP) or retinitis pigmentosa (RP), retinal ganglion degeneration, macular degeneration, hereditary optic neuropathy, metabolic optic neuropathy, optic neuropathy due to a toxic agent and neuropathy caused by adverse drug reactions or a vitamin deficiency.

79. The method or use of any one of claims 64-78, wherein the RTP801 inhibitor or the REDD2 inhibitor is administered simultaneously with the Casp2 inhibitor.

80. The method or use of any one of claims 64-78, wherein the RTP801 inhibitor or the REDD2 inhibitor is administered prior to administration of the Casp2 inhibitor.

81. The method or use of any one of claims 64-78, wherein the RTP801 inhibitor or the REDD2 inhibitor is administered subsequent to administration of the Casp2 inhibitor.

82. The method or use of any one of claims 64-81, wherein the RTP801 inhibitor or the REDD2 inhibitor; and the Casp2 inhibitor are configured for administration in different doses.

83. The method or use of claim 82, wherein the RTP801 inhibitor is a double-stranded RNA compound, wherein the Casp2 inhibitor is a double-stranded RNA compound;
and wherein the RTP801 inhibitor and the Casp2 inhibitor are administered in a ratio from 1:1 to 1000:1 RTP801 inhibitor:Casp2 inhibitor.

84. A method of providing neuroprotection to a neuron in a subject in need thereof, comprising administering to the subject an RTP801 inhibitor or a REDD2 inhibitor; and a Casp2 inhibitor; so as to thereby provide neuroprotection to the neuron in the subject.

85. Use of an RTP801 inhibitor or a REDD2 inhibitor; and a Casp2 inhibitor in the manufacture of a medicament for providing neuroprotection to a neuron in a subject in need thereof.

86. The method or use of claim 84 or 85, wherein the neuron is, or is comprised within, a system selected from the group consisting of a peripheral nervous system, a central nervous system, and an audio-vestibular system.

87. The method or use of claim 86, wherein the neuron is, or is comprised within, a visual system of a central nervous system.

88. The method or use of claim 87, wherein the neuron is a ganglion cell.

89. The method or use of claim 88, wherein the ganglion cell is selected from the group consisting of a retinal ganglion cell, a spiral ganglion cell, a vestibular ganglion cell, dorsal root ganglion and a peripheral ganglion cell.

90. The method or use of any one of claims 84-89, wherein the neuroprotection comprises protecting the neuron from death.

91. The method or use of claim 90, wherein death of the neuron is associated with one or more of a disease or disorder, a surgery, ischemia, ischemia/reperfusion, physical/mechanical trauma, a chemical agent, an infectious agent, an immunologic reaction and a nutritional imbalance.

92. A method of promoting neurite outgrowth, axonal regeneration or neural regeneration comprising contacting a neuron with an effective amount of an RTP801 inhibitor or a salt thereof or of a REDD2 inhibitor or a salt thereof, thereby promoting neurite outgrowth, axonal regeneration or neural regeneration.

93. A method of promoting neurite outgrowth, axonal regeneration or neural regeneration in a subject in need thereof, comprising administering to the subject a therapeutically effective amount of a RTP801 inhibitor or a pharmaceutically acceptable salt thereof; or a therapeutically effective amount of a REDD2 inhibitor or a pharmaceutically acceptable salt thereof, thereby promoting neurite outgrowth, axonal regeneration or neural regeneration in the subject.

94. A method for maintaining the viability of a neuron in a peripheral nervous system and/or a central nervous system, including a visual system, and/or an audio-vestibular system, comprising contacting the neuron with an RTP801 inhibitor or with a REDD2 inhibitor, thereby maintaining the viability of a neuron in the central nervous system, the visual system and/or the vestibular system.

95. A method of preventing, treating, or reducing symptoms of nerve injury in a subject, wherein the method comprises administering to the subject an effective amount of an agent that reduces the expression or activity of RTP801 or of REDD2, to prevent, treat, or reduce symptoms of nerve injury.

96. A method of treating a subject suffering nerve damage comprising the step of administering a composition comprising an RTP801 inhibitor or a REDD2 inhibitor to the subject, thereby treating the nerve damage in the subject.

97. Use of an RTP801 inhibitor or a salt thereof or of a REDD2 inhibitor or a salt thereof in the manufacture of a medicament for promoting neurite outgrowth, axonal regeneration or neural regeneration.

98. Use of an RTP801 inhibitor or a REDD2 inhibitor in the manufacture of a medicament for maintaining the viability of a neuron in a peripheral nervous system and/or a central nervous system, including a visual system, and/or an audio-vestibular system.

99. Use of an RTP801 inhibitor or a REDD2 inhibitor in the manufacture of a medicament for preventing, treating, or reducing symptoms of nerve injury in a subject.

100. Use of an RTP801 inhibitor or a REDD2 inhibitor in the manufacture of a medicament for treating a subject suffering nerve damage.

101. The method or use of any one of claims 92-100, wherein the RTP801 inhibitor or the REDD2 inhibitor is selected from the group consisting of an antibody, a polypeptide, a peptide, a nucleic acid molecule and a small organic molecule.

102. The method or use of claim 101, wherein the RTP801 inhibitor or the REDD2 inhibitor is a nucleic acid molecule.

103. The method or use of claim 102, wherein the nucleic acid molecule is a double-stranded RNA (dsRNA) compound.

104. The method or use of claim 103, wherein the double-stranded RNA compound comprises an antisense strand and a sense strand.

105. The method or use of claim 104, wherein the antisense strand of the RTP801 inhibitor double-stranded RNA compound comprises the sequence:
(SEQ ID NO: 7)
5′ AGCUGCAUCAGGUUGGCAC 3′.

106. The method or use of claim 105, wherein the RTP801 double-stranded RNA compound has the structure:
(antisense strand; SEQ ID NO: 9)
5′ AGCUGCAUCAGGUUGGCAC 3′
(sense strand, SEQ ID NO: 10)
3′ UCGACGUAGUCCAACCGUG 5′.

107. The method or use of any of claim 92, 93, 94 or 97-98, wherein the neuron is, or is comprised within a system selected from a peripheral nervous system and a central nervous system, and an audio-vestibular system.

108. The method or use of claim 107, wherein the neuron is, or is comprised within, a visual system of a central nervous system.

109. The method or use of any of claim 92, 93, 94 or 97-98, wherein the neuron is a ganglion cell.

110. The method or use of claim 109, wherein the ganglion cell is selected from the group consisting of a retinal ganglion cell, a spiral ganglion cell, a vestibular ganglion cell, dorsal root ganglion and a peripheral ganglion cell.

111. The method or use of any of claim 92, 93, 94 or 97-98, wherein the neuron is derived from a stem cell or from a progenitor cell.

112. The method or use of claim 111, wherein the stem cell is Muller's glia.
